(19) United States
(12) Patent Application Publication
(10) Pub. No.: US 2026/0177553 A1
(43) Pub. Date: Jun. 25, 2026

(54) Expanded metabolic pathway for androgen production by commensal bacteria

(71) Applicant: The Brd. of Trustees of the Univ. of Illinois, Urbana, IL (US)

(72) Inventor: Jason M. Ridlon, Savoy, IL (US)

(73) Assignee: The Brd. of Trustees of the Univ. of Illinois, Urbana, IL (US)

(21) Appl. No.: 19/375,980

(22) Filed: Oct. 31, 2025

Related U.S. Application Data
(60) Provisional application No. 63/715,232, filed on Nov. 1, 2024.

(51) Int. Cl. G01N 33/57555; C12N 9/04; C12P 33/00; G01N 33/573; G01N 33/575

(52) U.S. Cl. CPC G01N 33/57555; C12N 9/0006; C12P 33/00; C12Y 101/01146; G01N 33/573; G01N 33/57585; G01N 2333/902

(57) ABSTRACT

Provided herein is the first commensal microbial gene encoding an enzyme that catalyzes the conversion of androstenedione to epitestosterone in the gut bacterium Clostridium scindens (desF). Also provided is an important and unrecognized capability for epitestosterone in androgen receptor-dependent prostate cancer cell proliferation and the potential clinical relevance of this bacterial enzymatic pathway (desF) in prostate cancer. Additionally, it was discovered that bacterial isolates from urine or prostatectomy tissue are capable of androgen production and that urinary tract bacteria can exhibit 17β-hydroxysteroid dehydrogenase activity The gene in urinary isolates encoding 17β-hydroxysteroid dehydrogenase that catalyzes the conversion of androstenedione to testosterone was identified and named desG.

### PRIORITY

This application claims the benefit of U.S. Ser. No. 63/715,232, filed on Nov. 1, 2024, which is incorporated by reference herein in its entirety.

### GOVERNMENT FUNDING

This invention was made with government support under 1R01GM145920-01 awarded by the National Institutes of Health. The United States Government has certain rights in the invention.

### SEQUENCE LISTING

The specification incorporates by reference a sequence listing identified as 769893-UIUC-055.xlm, which is 15,411 bytes in size and was created on 29 Jan. 2026.

### BACKGROUND

A growing body of literature implicates commensal microbiota in the modulation of circulating androgen levels in the host, which could have far-reaching implications for androgen-mediated diseases. However, the microbial genetic pathways involved in androgen production remain unknown.

### SUMMARY

Provided herein is an isolated nucleic acid molecule comprising: a first polynucleotide as set forth in SEQ ID NO:1 or SEQ ID NO:3, wherein the first polynucleotide optionally comprises less than 770 nucleic acids; and (i) a second heterologous polynucleotide; or (ii) a detectable label. The second heterologous polynucleotide can encode a marker, a label, or purification tag. The second heterologous polynucleotide can comprise a heterologous expression control sequence.

Another aspect provides an isolated nucleic acid molecule comprising a sequence set forth in SEQ ID NO:1 or SEQ ID NO:3 and containing 1 to 40 substitution modifications relative to SEQ ID NO:1 or SEQ ID NO:3. The substitution modifications can be conservative amino acid substitution modifications or semi-conservative substitution modification, or combinations thereof.

Yet another aspect provides an expression cassette comprising a first polynucleotide as set forth in SEQ ID NO:1, wherein the first polynucleotide optionally comprises less than 770 nucleic acids and a second polynucleotide comprising at least one expression control sequence.

Even another aspect provides an expression cassette comprising a first polynucleotide as set forth in SEQ ID NO:3, wherein the first polynucleotide optionally comprises less than 770 nucleic acids and a second polynucleotide comprising at least one expression control sequence. The expression control sequence can be a promoter operably linked to the first polynucleotide.

An aspect provides a vector comprising any of the expression cassettes described herein.

Another aspect provides an isolated polypeptide comprising a sequence set forth in SEQ ID NO:2 or SEQ ID NO:4 and containing 1 to 20 amino acid substitution modifications relative to SEQ ID NO:2 or SEQ ID NO:4. The polypeptide can have dehydrogenase 17α-HSDH activity (SEQ ID NO:2) or 17β-HSDH activity (SEQ ID NO:4). The amino acid substitution modifications can be conservative amino acid substitution modifications or semi-conservative substitution modification, or combinations thereof. The polypeptide can comprise less than 260 amino acids.

Yet another aspect provides an isolated polypeptide comprising a sequence set forth in SEQ ID NO:2 or SEQ ID NO:4 and an indicator reagent, an amino acid spacer, an amino acid linker, a signal sequence, a stop transfer sequence, a transmembrane domain, a protein purification ligand, an affinity purification tag, a heterologous polypeptide, or a combination thereof.

Even another aspect provides a fusion protein comprising a sequence set forth in SEQ ID NO:2 or SEQ ID NO:4 and a heterologous polypeptide.

Another aspect provides a recombinant cell comprising a polynucleotide as set forth in SEQ ID NO:1 and/or SEQ ID NO:3. The polynucleotide as set forth in SEQ ID NO:1 can be less than 770 nucleic acids in length. The polynucleotide as set forth in SEQ ID NO:3 can be less than 770 nucleic acids in length. The recombinant cell can be a bacterial cell, a fungal cell, or a eukaryotic cell. Also provided is a method of producing 17α-hydroxysteroid dehydrogenase (17α-HSDH) comprising culturing the recombinant cells and recovering 17α-hydroxysteroid dehydrogenase or producing 17β-hydroxysteroid dehydrogenase (17β-HSDH) comprising culturing the recombinant cells and recovering 17β-hydroxysteroid dehydrogenase. Epitestosterone can be made by contacting the recombinant cells with androstenedione and recovering epitestosterone. A recombinant cell can express androstenedione naturally or recombinantly. A method of producing testosterone is provided comprising contacting the recombinant cells with androstenedione and recovering testosterone. The recombinant cell can express androstenedione naturally or recombinantly.

An aspect provides a method of identifying prostate cancer, resistant prostate cancer, or advancing prostate cancer in a patient comprising detecting a level of 17α-hydroxysteroid dehydrogenase (17α-HSDH) present in a prostatectomy sample, a urine sample, or a fecal sample of the patient, wherein an elevated level of 17α-HSDH as compared to a control sample or standard indicates prostate cancer, resistant prostate cancer, or advancing prostate cancer. The 17α-hydroxysteroid dehydrogenase can be as set forth in SEQ ID NO:2.

Another aspect provides a method of identifying prostate cancer, resistant prostate cancer, or advancing prostate cancer in a patient comprising detecting a level of 17β-hydroxysteroid dehydrogenase (17β-HSDH) present in a prostatectomy sample, a urine sample, or a fecal sample of the patient, wherein an elevated level of 17β-HSDH as compared to a control sample or standard indicates prostate cancer, resistant prostate cancer, or advancing prostate cancer. The 17 β-hydroxysteroid dehydrogenase can be as set forth in SEQ ID NO:4. The patient can be treated for prostate cancer by prostatectomy, hormone therapy, active surveillance, radiation therapy, high-intensity focused ultrasound, cryotherapy, chemotherapy, immunotherapy, and/or bisphosphonate therapy.

Yet another aspect is a method for inducing proliferation of cancer cells comprising adding about 5-20 nM epiT to a cancer cell culture.

An aspect provides a method of treatment of prostate cancer comprising administering antibiotics effective against bacteria expressing desAB, desG, and/or desF and/or an inhibitor of desAB, desG, and/or desF to a prostate cancer patient in need thereof.

Another aspect provides a method of monitoring prostate cancer in a subject comprising detecting a level of 17α-HSDH and/or 17β-HSDH in a sample from the subject and comparing the level to a control sample or control at two or more time points; and comparing results at the two or more points such that the prostate cancer is monitored in the subject.

### BRIEF DESCRIPTION OF THE DRAWINGS

FIG. 1A-1I show identification of the desF gene encoding steroid 17α-hydroxysteroid dehydrogenase involved in epitestosterone formation by Clostridium scindens. 1A. proposed biochemical pathway by which Csci35704 and Csci12708 convert 11DC to AD and epiT. SEM images of Csci35704 and Csci12708 are included. 1B. LC/MS/MS chromatographs of 11DC, AD, and epiT at time 0 and 24 h (left) and quantification of metabolites (right) (n=3). 1C. Venn diagram summarizing comparative genomic analysis between Csci35704 and Csci12708 (see FIG. 7 for reductases unique to Csci12708). 1D. Scatterplot of RNA-Seq analysis to identify genes upregulated by 50 μM 11OHAD in Csci12708. Significantly upregulated genes (>0.58 log₂ fold; <0.05 FDR) in red, downregulated genes in blue, and not differentially regulated in black. 1E. Gene organization of 17α-hydroxysteroid dehydrogenase candidate (“desF”) identified in RNA-Seq analysis. 1F. SDS-PAGE of Streptavidin-purified recombinant DesF and LC/MS/MS chromatographs showing NADPH-dependent conversion of AD to epiT at 0 and 24 h, and NADP⁺-dependent conversion of epiT to AD at 0 h and 24 h. 1G. Ribbon diagram of AlphaFold 2 structural prediction of DesF. NADP⁺ (crosshatching on right) and epitestosterone (crosshatching on left) are depicted as space-filling models, and were aligned and placed with VMD and ligand-structure interactions were minimized using NAMD through its QuikMD interface. 1H. Space-filling model of both the DesF (gray) and ligands NADP⁺ (crosshatching on the right) and epitestosterone (crosshatching on the left). I. Molecular dynamic trajectory analysis revealed strong interactions between ligands and catalytic triad. SER144 and TYR157 form stable hydrogen bonds with epitestosterone, while LYS161 was identified as key in NADP⁺ stabilization.

FIG. 2A-2H show epitestosterone and gut microbial desF in prostate cancer. 2A. Schematic representation of in vitro experiments examining dose and time dependent growth of LNCaP cells in the presence of androgen candidates as well as determination of the effect of androgen receptor antagonist, enzalutamide. 2B. Dose-dependent proliferation (MTS assay) of LNCaP cells in the presence of androstenedione (AD), testosterone (T), epitestosterone (epiT), and vehicle control (VC; methanol 0.5% v/v) at 1.0 nM or 10.0 nM (n=6). 2C. Time-dependent proliferation (MTS assay) of LNCaP cells in the presence (2 μM) or absence of AR-antagonist, enzalutamide. Proliferation was measured at 24, 48, or 96 h in the presence of 10 nM steroids or VC (n=6). 2D. qRT-PCR quantification of KLK3 gene (encoding prostate specific antigen, PSA) after treatment with 10 nM T, epiT, or VC at 24, 48, 96 h in the presence of absence of 2 μM enzalutamide (n=3). 2E. Schematic representation of clinical study examining fecal desF quantification (qPCR) in advanced prostate cancer patients undergoing treatment with AA/P either stable (blood PSA trend stable) (n=28) or progressing (blood PSA trend increasing) (n=28). 2F. Scatterplots of desF % normalized to total fecal 16S rRNA gene (left) or desF copy number/10 ng DNA in stable vs. progressing male patients. 2G. Proportion of stable vs. progressing patients above a threshold of 40 desF copies vs. below 40 desF copies. 2H. Fecal desF quantification (n=12) of donor samples taken both while they were stable on AA/P and when they were progressing. P values were calculated by unpaired t-test and Benjamini-Hochberg correction, * P<0.05, ** P<0.01,*** P<0.001, **** P<0.0001. Clinical data significance was determined by Chi squared analysis.

FIG. 3A-3K shows androgen-producing bacterium isolated from human prostatectomy tissue expresses a multi-step pathway for conversion of glucocorticoids to derivatives of testosterone. 3A-3C. A post-surgical prostatectomy specimen was placed in a sterile container following resection and the biopsy tissues were minced in sterile PBS, placed in a tube of thioglycolate broth, and cultured anaerobically at 37° C. A single colony capable of converting cortisol to 11OHAD and 11OHT was identified by LC/MS/MS. SEM imaging of the strain was obtained and the complete genome was sequenced. The isolate was identified as Propionimicrobium lymphophilum and was named “androgen-producing isolate 1” (API-1). 3D. A gene predicted to encode “3β-hydroxycholanate dehydrogenase” was identified in the genome sequence and selected as a potential 17β-HSDH candidate. 3E. We cloned ILDKDCJM_00716 into pET51b(+) and overexpressed and Streptactin-purified the recombinant protein for enzyme assay. 3F. LC/MS/MS analysis of steroid products after incubation of ILDKDCJM_00716, 200 μM NADPH and 50 μM 11OHAD. 11OHT formation was observed after 6 h incubation. We proposed the name desG for ILDKDCJM_00716. 3G. Pathway for conversion of cortisol to 11OHAD and 11OHT by P. lymphophilum API-1. 3H. Ribbon diagram of AlphaFold 2 structural prediction of DesF. NADP⁺ (crosshatching on the right) and testosterone (crosshatching on the left) are depicted as space-filling models and were aligned and placed with VMD and ligand-structure interactions were minimized using NAMD through its QuikMD interface. 3I. Space-filling model of both the DesG (gray) and ligands NADP⁺ (crosshatching on the right) and testosterone (crosshatching on the left). 3J. Molecular dynamic trajectory analysis revealed strong interactions between ligands and catalytic triad. SER144 and TYR157 form stable hydrogen bonds with testosterone, while LYS161 was identified as key in NADP⁺ stabilization. 3K. The product of cortisol metabolism by strains API-1 and API-2, 11OHT, causes significant (P<0.001) and prolonged (96 h) proliferation of LNCaP cells relative to vehicle control (VC; methanol 0.5% v/v). P values were calculated by unpaired t-test and Benjamini-Hochberg correction, * P<0.05, ** P<0.01, *** P<0.001.

FIG. 4A-4G. show urinary tract isolates of Propionimicrobium lymphophilum drives prostate cancer cell growth through androgen-production. 4A. Strain API-1 was isolated from prostatectomy tissue from a male with mCRCP, and strain API-2 was isolated ˜17 years later from a urine sample collected from the same patient. 4B. LC/MS analysis confirmed the conversion of cortisol to 11OHAD and 11OHT in encapsulated beads (see 4E, 4F). 4C, 4D. Synteny and comparison of gene content between the two strains indicates a high degree of similarity. 4E. Schematic representation of urinary tract isolates encapsulated in microgels which are co-cultured in culture medium with LNCaP cells in the presence of cortisol. 4F. Micrographs of calcium alginate microgels at Day 0 and Day 2 display dense growth of API-2 in DMEM medium under aerobic conditions. 4G. LNCaP proliferation in the presence/absence of API-2 and/or 10 nM cortisol (n=4). P values were calculated by unpaired t-test and Benjamini-Hochberg correction, * P<0.05, ** P<0.01, *** P<0.001.

FIG. 5A-5D show isolation and characterization of androgen-producing bacteria from human male urine samples. 5A. Schematic of isolation and screening approach from human male urine 5B. Biochemical pathway and DesABEG enzyme functions proposed for urinary tract isolates. 5C-5D. Organization of desABE and desG genes corresponds with the formation of 11OHAD and 11HT in pure cultures incubated with 50 μM cortisol.

FIG. 6 shows proton and carbon NMR analysis of purified reaction product of androstenedione in cultures of Clostridium scindens VP112708. ¹H and ¹³C-NMR spectroscopic data were obtained on a JNM-ECA800 (JEOL, Ltd., Tokyo, Japan) instrument operated at 800 and 200 MHz, respectively, with CDCl₃ as the NMR solvent. Chemical shifts were expressed in d (ppm), and coupling constants J_{H,H} are given in Hz. ¹H-1H nuclear overhauser effect spectroscopy (NOESY, ¹H-1H correlation spectroscopy (COSY), ¹H-¹³C heteronuclear single-quantum correlation spectroscopy (HSQC), and ¹H-¹³C heteronuclear multiple-bond correlation spectroscopy (HMBC) spectra were obtained using gradient-selected pulse sequences. The ¹³C distortionless enhancement by polarization transfer (135°, 90°, and 45°) spectra were measured between CH₃, CH₂, CH, and coherence based on their proton environments. ¹H NMR (800 MHz) δ: 0.71 (3H, s, H-18), 0.98 (1H, ddd, J=12.8, 11.2, 4.0, H-9), 1.10 (1H, dddd, J=14.4, 12.8, 12.0, 4.0, H-7a), 1.19 (3H, s, H-19), 1.22 (1H, dddd, J=12.0, 11.2, 11.2, 6.4, H-15a), 1.42 (1H, J=12.0, 11.2, 7.2, H-14), 1.478 (1H, dddd-like, J=12.8, 12.8, 12.8, 4.8, H-11a), 1.484 (1H, ddd, J=15.2, 10.4, 6.4, H-16a), 1.52 (1H, ddd-like, J=12.8, 4.8, 3.2, H-12a), 1.54 (1H, dddd-like, J=12.0, 12.0, 11.2, 4.0, H-8), 1.57 (1H, ddd-like, J=12.8, 12.8, 4.0, H-12b), 1.65 (1H, dddd-like, J=12.8, 4.0, 4.0, 3.2 H-11b), 1.72 (1H, ddd-like, J=14.4, 13.6, 4.8, H-1a), 1.79 (1H, dddd, J=12.0, 10.4, 7.2, 2.4, H-15b), 1.88 (1H, J=dddd, J=12.8, 4.8, 4.0, 2.4, H-7b), 2.04 (1H, ddd, J=13.6, 4.8, 3.2, H-1b), 2.18 (dddd, J=15.2, 11.2, 5.6, 2.4, H-16b), 2.28 (1H, ddd, J=14.4, 4.0, 2.4, H-6a), 2.34 (1H, dddd, J=16.8, 4.8, 3.2, 0.8, H-2a), 2.39 (1H, dddd, J=14.4, 14.4, 4.8, 1.6, H-6b), 2.42 (1H, ddd, J=16.8, 14.4, 4.8, H-2b), 3.77 (1H, d, J=5.6, H-17), 5.74 (1H, dd-like, J=1.6, 0.8, H-4). ¹³C NMR (200 MHz) δ: 16.9 (C-18), 17.4 (C-19), 20.6 (C-11), 24.6 (C-15), 31.2 (C-12), 32.3 (C-7), 32.4 (C-16), 32.9 (C-6), 33.9 (C-2), 35.8 (C-1), 35.9 (C-8), 38.7 (C-10), 45.1 (C-13), 48.2 (C-14), 53.6 (C-9), 79.7 (C-17), 123.9 (C-4), 171.3 (C-5), 199.5 (C-3).

| | |
|---|---|
| H-1ax | 1.72 (1H, ddd-like, J = 14.4, 13.6, 4.8, H-1a) |
| H-1eq | 2.04 (1H, ddd, J = 13.6, 4.8, 3.2, H-1b) |
| H-2ax | 2.42 (1H, ddd, J = 16.8, 14.4, 4.8, H-2b) |
| H-2eq | 2.34 (1H, dddd, J = 16.8, 4.8, 3.2, 0.8, H-2a) |
| H-4 | 5.74 (1H, dd-like, J = 1.6, 0.8, H-4) |
| H-6ax | 2.39 (1H, dddd, J = 14.4, 14.4, 4.8, 1.6, H-6b) |
| H-6eq | 2.28 (1H, ddd, J = 14.4, 4.0, 2.4, H-6a) |
| H-7ax | 1.10 (1H, dddd, J = 14.4, 12.8, 12.0, 4.0, H-7a) |
| H-7eq | 1.88 (1H, J = dddd, J = 12.8, 4.8, 4.0, 2.4, H-7b) |
| H-8 | 1.54 (1H, dddd-like, J = 12.0, 12.0, 11.2, 4.0, H-8), |
| H-9 | 0.98 (1H, ddd, J = 12.8, 11.2, 4.0, H-9), |
| H-11ax | 1.478 (1H, dddd-like, J = 12.8, 12.8, 12.8, 4.8, H-11a) |
| H-11eq | 1.65 (1H, dddd-like, J = 12.8, 4.0, 4.0, 3.2 H-11b), |
| H-12ax | 1.57 (1H, ddd-like, J = 12.8, 12.8, 4.0, H-12b) |
| H-12eq | 1.52 (1H, ddd-like, J = 12.8, 4.8, 3.2, H-12a) |
| H-14 | 1.42 (1H, J = 12.0, 11.2, 7.2, H-14) |
| H-15a | 1.22 (1H, dddd, J = 12.0, 11.2, 11.2, 6.4, H-15a), |
| H-15b | 1.79 (1H, dddd, J = 12.0, 10.4, 7.2, 2.4, H-15b), |
| H-16a | 1.484 (1H, ddd, J = 15.2, 10.4, 6.4, H-16a) |
| H-16b | 2.18 (dddd, J = 15.2, 11.2, 5.6, 2.4, H-16b), |
| H-17 | 3.77 (1H, d, J = 5.6, H-17) |
| 18-CH3 | 0.71 (3H, s, H-18) |
| 19-CH3 | 1.19 (3H, s, H-19) |

| | Sample 17-epi-testosterone | Testosterone¹⁾ | Testosterone²⁾ |
|---|---|---|---|
| C1 | 35.8 | 36.1 | 35.6 |
| C2 | 33.9 | 34.1 | 33.8 |
| C3 | 199.5 | 198.0 | 199.4 |
| C4 | 123.9 | 124.2 | 123.6 |
| C5 | 171.3 | 170.4 | 171.0 |
| C6 | 32.9 | 32.8 | 32.7 |
| C7 | 32.3 | 32.2 | 31.5 |
| C8 | 35.9 | 36.1 | 35.0 |
| C9 | 53.6 | 54.6 | 53.9 |
| C10 | 38.7 | 39.0 | 38.6 |
| C11 | 20.6 | 21.2 | 20.6 |
| C12 | 31.2 | 37.1 | 36.4 |
| C13 | 45.1 | 43.2 | 42.7 |
| C14 | 48.2 | 51.1 | 50.4 |
| C15 | 24.6 | 23.8 | 23.2 |
| C16 | 32.4 | 30.7 | 30.1 |
| C17 | 79.7 | 81.3 | 81.0 |
| C18 | 16.9 | 11.3 | 11.0 |
| C19 | 17.4 | 17.3 | 17.3 |

¹⁾25.2 MHz [JOC, 46, 1127 (1981)]
²⁾25.2 MHz [JCS, Perkin 1, 1956 (1975)]

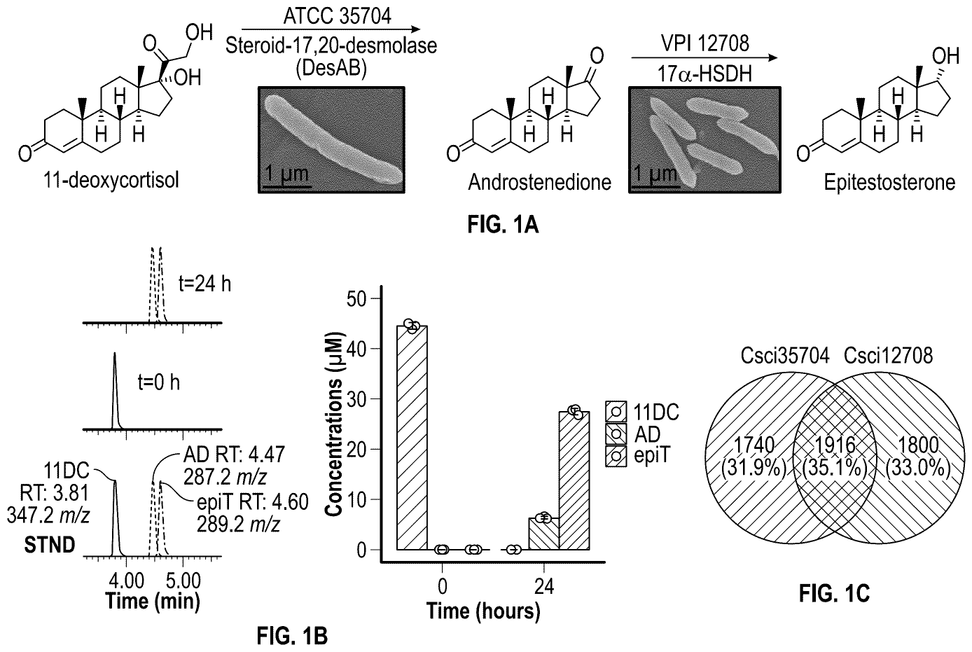

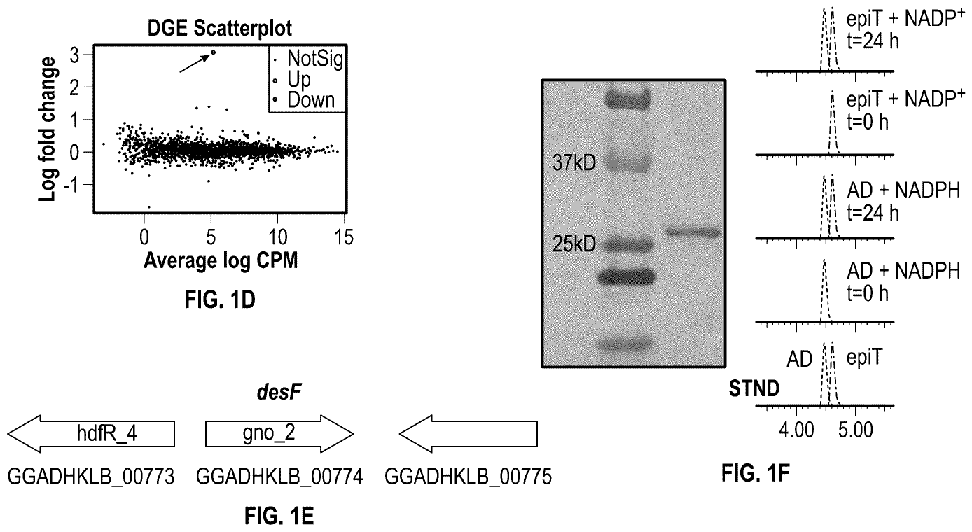

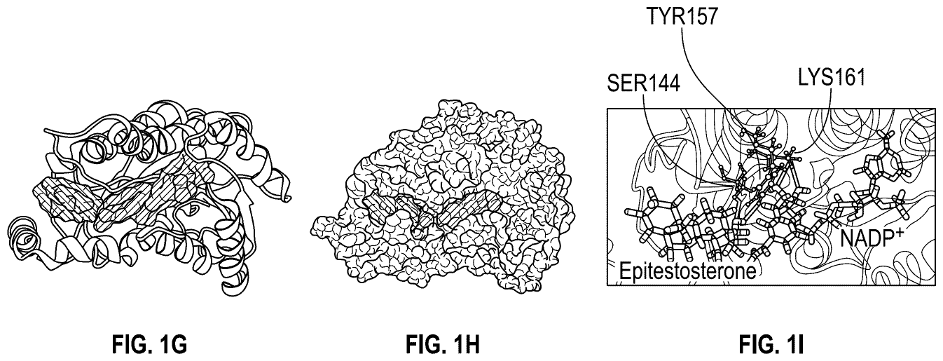

FIG. 7 shows comparative genome analysis of Csci35704 and Csci12708 revealed reductase candidates for 17α-HSDH. Highlighted in yellow is the candidate significantly upregulated in RNA-Seq dataset (see FIG. 1).

FIG. 8A-8G show RNA-seq analysis to identify the gene encoding 17 α-hydroxysteroid dehydrogenase involved in epitestosterone formation by Clostridium scindens. 8A. In vitro induction of the gene encoding 17α-hydroxysteroid dehydrogenase by addition of 50 μM 11OHAD. DMSO was used as the vehicle control (Experiment one, n=4). 8B. Percentages of the reads mapped to the reference genome of Clostridium scindens VPI 12708 (Experiment one). 8C. MDS plot showed the gene composition by in vitro induction of Experiment one. 8D. In vitro induction of the gene encoding 17α-hydroxysteroid dehydrogenase by addition of 50 μM 11OHAD (Experiment two, validation experiment, n=3). 8E. Percentages of the reads (Experiment two) mapped to the reference genome of Clostridium scindens VPI 12708. 8F. MDS plot showed the gene composition by in vitro induction (Experiment two). 8G. Differential gene expression scatter plot of Experiment two. Significantly upregulated genes in red, downregulated genes in blue, and not differentially regulated in black.

FIG. 9 shows maximum likelihood phylogeny of DesF. Tree was colored by taxonomic affiliation, according to the included key. Radial tree shows the complete one-thousand sequence tree, and the region near C. scindens is shown as a phylogram. Numbers near nodes are branch support values (SH-like approximate likelihood-ration test and ultrafast bootstrap), with only values greater than 50 shown (an * denotes that one of the values is under 50 but the other is not).

FIG. 10 shows phylogenomic and diversity of des gene presence in strains of C. scindens. The formation of two clades is shown, Clade 1 includes 15 strains and Clade 2 19 strains. Bootstrap support values above 50% are shown in stars at nodes. Dots show des gene presence in strains of each clade.

FIG. 11 shows steroid-dependent proliferation of VCaP cell line. Data are shown with mean±standard deviation (n=6). P values were calculated by unpaired t-test and Benjamini-Hochberg correction, * P<0.05, ** P<0.01,*** P<0.001. VC: Vehicle control (0.5% methanol v/v); AD: Androstenedione; T: Testosterone; epiT: Epi-testosterone; 11OHT: 11β-hydroxy-testosterone.

FIG. 12A-12E show Clostridium scindens generates androgenic derivative of epitestosterone from 1,4-androstadiene-3,11,17-trione (AT). 12A. In vitro incubation of Clostridium scindens VPI 12708 with 50 μM AT as substrate. The medium culture after removing the bacterial cells was diluted and used for the LNCaP cell line promotion effect study. 12B. A proposed biochemical pathway by which Csci12708 convert AT to epiAT. 12C. LC/MS chromatographs of AT and epiAT at time 0, 24, 48 and 72 h and the change of the under-curve area of AT and epiAT overtime (n=3). 12D. Dose-dependent proliferation of LNCaP cells in the presence of medium culture control (CL), medium culture control with AT (AT), and metabolite of AT in the medium culture (epiAT) at 0.1 nM, 1.0 nM and 10.0 nM (n=6). 12E. Enzalutamide effect on the LNCaP cell line proliferation in the presence of medium culture control (CL), medium culture control with AT (AT), and metabolite of AT in the medium culture (epiAT) at 10.0 nM (n=8). Data are shown with mean±standard deviation. P values were calculated by unpaired t-test and Benjamini-Hochberg correction, * P<0.05, ** P<0.01,*** P<0.001.

FIG. 13 shows bacterial colony morphology, SEM images and the circular genome maps of the androgen-producing Propionimicrobium lymphophilum strains from men diagnosed with prostate cancer and age-matched control males.

FIG. 14 shows maximum likelihood phylogenetic analysis of DesG from Propionimicrobium lymphophilum strain API-1. Tree was colored by taxonomic affiliation, according to the included key. Radial tree shows the complete five-hundred sequence tree, and the region near P. lymphophilum is shown as a phylogram. Numbers near nodes are branch support values (SH-like approximate likelihood-ration test and ultrafast bootstrap), with only values greater than 50 shown (an * denotes that one of the values is under 50 but the other is not).

FIG. 15A-15D show functional sampling of desG homologs for 17β-HSDH activity. 15A. Zoom in of DesG branch with proteins chosen for functional characterization (asterisks). 15B. Genomic context of desG candidates from taxa represented in DesG branch. 15C. SDS-PAGE of affinity (Streptactin) purified recombinant DesG candidates. 15D. LC/MS traces after enzyme assay containing either DesG candidate (10 nM)+NADPH (200 μM) and androstenedione (50 μM), or DesG candidate (10 nM)+NADP+ (200 μM) and testosterone (50 μM) incubated for 6 h before steroid extraction. Authentic androstenedione and testosterone standards were also included.

FIG. 16A-16C. show genome quality metrics (A.), average nucleotide identity, (B.) and synteny between Propionimicrobium lymphophilum strains (C.) isolated from human male urine.

FIG. 17A-17C show Abiraterone (A) and abiraterone acetate (AA) are not inhibitors of bacterial steroid-17,20-desmolase (desAB). 17A. P. lymphophilium API-1 treated with 50 μM AA and then incubated 50 μM cortisol for 48 hours. 17B. API-1 strain treated with AA (1 μM and cortisol or prednisone (50 μM). (17C.) API-1 treated with 50 μM A and then incubated with 50 μM cortisol for 48 hours.

FIG. 18 shows schematic representation of potential host-microbiome interactions relating to the conversion of glucocorticoids to androgens.

### DETAILED DESCRIPTION

Provided herein is the first commensal microbial gene encoding an enzyme that catalyzes the conversion of androstenedione to epitestosterone in the gut bacterium Clostridium scindens (desF). Also provided is an important and unrecognized capability for epitestosterone in androgen receptor-dependent prostate cancer cell proliferation and the potential clinical relevance of this bacterial enzymatic pathway (desF) in prostate cancer. Additionally, it was discovered that bacterial isolates from urine or prostatectomy tissue are capable of androgen production and that urinary tract bacteria can exhibit 17β-hydroxysteroid dehydrogenase activity. The gene in urinary isolates encoding 17β-hydroxysteroid dehydrogenase that catalyzes the conversion of androstenedione to testosterone was identified and named desG. Urinary androgen-producing bacterial strains are capable of promoting prostate cancer cell growth through steroid metabolism. The structures and ligand binding to DesF and DesG are provided herein.

Human microbiome research has revealed that the human body is composed numerically of equal bacterial and mammalian cells¹, and that 99% of the functional genes are microbial². Accordingly, the functional capacity of the human-associated microbiota to influence human health and disease is immense. Host-associated microbial communities can be significant contributors to circulating androgens, and gut androgen production may influence androgen-mediated disease. The bacterial enzymatic pathways that mediate gut androgen production are largely unknown, and elucidation of these pathways as well as their demonstrated relevance to androgen-mediated disease could offer novel approaches for therapeutic targeting.

The first gut bacterial enzymatic pathway (desABC operon) that is involved in the conversion of cortisol derivatives to 11-oxy-androgens in Clostridium scindens^{6,7} is provided herein. The desC gene encodes steroid 20α-hydroxysteroid dehydrogenase (20α-HSDH) involved in side-chain oxidoreduction^{6,8}, while desAB encodes steroid-17,20-desmolase involved in side-chain cleavage of cortisol derivatives to 11-oxy-androgens^{6,7}. 11-oxy-androgens are now regarded as potent androgen receptor (AR) ligands (e.g., 11-keto-T and 11-keto-dihydrotestosterone (11-keto-DHT) on par with T and DHT⁹. Androgen production in the gut via bacterial species that carry the des operon may contribute to disease etiology, with prostate cancer as one exemplary paradigm. mCRPC is largely incurable and characterized by progressive metastatic cancer growth despite treatment that blocks androgen synthesis (e.g., gonadotropin-releasing hormone agonist/antagonist) in the testes. Second-line treatments that block adrenal androgen synthesis (e.g., abiraterone acetate (AA) given with the replacement glucocorticoid prednisone (P)), and/or directly antagonize AR (e.g., enzalutamide) are likewise not curative, and resistance invariably develops¹⁰. Despite castrate levels of circulating T in individuals with mCRPC, intra-tumoral levels of androgens remain high^{11,12} Current research on the source(s) of these intra-tumoral androgens is almost entirely focused on host enzymatic biosynthesis and intracrine pathways through which androgen-precursors are synthesized and become AR-ligands¹³⁻¹⁶. Despite these efforts, the source of intra-tumoral androgens that may counteract therapeutic castration is unclear. Furthermore, the capacity for androgen production by microbiota colonizing other host epithelial sites to contribute to disease etiology and therapeutic response is completely unexplored.

Provided herein is an expansion of the bacterial desmolase pathway in desAB harboring taxa to include host-associated bacteria expressing 17α-hydroxysteroid dehydrogenase (17α-HSDH; desF) or 17β-HSDH (desG) involved in the formation of epitestosterone (epiT) and T, respectively. Additionally, contrary to the current dogma, epiT is an androgen receptor agonist that drives prolonged expression of kallikrein-3 (KLK3, also known as prostate specific antigen or PSA) and proliferation of prostate cancer cell lines. Fecal desF-carrying bacteria are present in the gut microbiota of individuals with advanced prostate cancer, and fecal desF levels are elevated in individuals with disease progression on androgen-deprivation therapy combined with AA/P versus those who are responding. Strains of P. lymphophilum from human prostatectomy tissue and urine are capable of converting cortisol to AR ligands that drive hormone-responsive prostate cancer cell proliferation in vitro using a unique microencapsulation technique. These findings significantly expand our knowledge of human steroid microbiology.

### Polynucleotides and Genes

Polynucleotides contain less than an entire microbial genome and can be single- or double-stranded nucleic acids. A polynucleotide can be RNA, DNA, cDNA, genomic DNA, chemically synthesized RNA or DNA or combinations thereof. A polynucleotide can comprise, for example, a gene, open reading frame, non-coding region, or regulatory element.

A gene is any polynucleotide molecule that encodes a polypeptide, protein, or fragment thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source, but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., SEQ ID NO:2 or 4) or variants thereof).

Polynucleotides can comprise other nucleotide sequences, such as sequences coding for linkers, signal sequences, stop transfer sequences, transmembrane domains, or ligands useful in protein purification such as glutathione-S-transferase, histidine tag, and Staphylococcal protein A.

Polynucleotides can be codon optimized for expression in bacteria or other suitable host cell. Codon optimization is the process of modifying the coding region of a gene to more closely align the codon usage of a gene of interest with the codon usage frequency or codon bias of the target cell or organism, while retaining the same amino acid coding sequence. In some instances, codon optimization may improve translation efficiency. Numerous codon usage tables are publicly available and may be found, for example at genscript.com/tools/codon-frequency-tablem or kazusa.or.jp/codon/. See also Athey et al., A new and updated resource for codon usage tables, BMC Bioinformatics. 2017; 18:391 (2017).

Polynucleotides can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Species homologs and variants of polynucleotides that encode biologically functional polypeptides also are polynucleotides.

In an aspect, a polynucleotide is considered to be an equivalent to SEQ ID NO:2 or 4 if it has 90% or more sequence identity to SEQ ID NO:2 or 4 and the polypeptide expressed by the polynucleotide has 90 to 110% of the biological activity (i.e., steroid 17 α-hydroxysteroid dehydrogenase activity or 17β-hydroxysteroid dehydrogenase activity) of the wild-type protein.

Polynucleotides can be obtained from nucleic acid sequences present in, for example, a microorganism such as a bacterium. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise coding sequences for naturally occurring polypeptides or can encode altered sequences that do not occur in nature.

Unless otherwise indicated, the term polynucleotide or gene includes reference to the specified sequence as well as the complementary sequence thereof.

An aspect provides an isolated nucleic acid molecule comprising SEQ ID NO:1 or SEQ ID NO:3. Also provided is a first polynucleotide as set forth in SEQ ID NO:1 or SEQ ID NO:3, wherein the first polynucleotide comprises less than 900 nucleic acids (e.g., about less than 900, 850, 800, 790, 780, 775, 770, 768, 765, or 760 nucleic acids); and (i) a second heterologous polynucleotide; or (ii) a detectable label. The second heterologous polynucleotide can encode a marker, a label, or purification tag. Markers and detectable labels are polynucleotides, polypeptides, compounds, and/or elements that can be detected due to their specific functional properties, and/or chemical characteristics, the use of which allows an to be detected, and/or further quantified if desired. Examples of detectable labels include, but not limited to, radioactive isotopes, fluorescent molecules, semiconductor nanocrystals, chemiluminescent molecules, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Detectable labels or markers can be encoded by a polynucleotide, linked to a polynucleotide, or otherwise associated with a polynucleotide. Detectable labels or markers can be linked to a polypeptide or be part of fusion protein, or otherwise associate with a polypeptide. The second heterologous polynucleotide can comprise a heterologous expression control sequence.

An isolated nucleic acid molecule can comprise a sequence set forth in SEQ ID NO:1 or SEQ ID NO:3 and containing 1 to 70 (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 333, 34, 35, 36, 37, 38, 39, 40, 50, 60, 70 or more substitution modifications relative to SEQ ID NO:1 or SEQ ID NO:3; or 70, 60, 50, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or less substitution modifications relative to SEQ ID NO:1 or SEQ ID NO:3). In an aspect, a substitution modification replaces one more nucleotides with other nucleotides that encode the same amino acid as the non-modified polynucleotide. In an aspect, a substitution modification replaces one or more nucleotides with other nucleotides that encode a different amino acid as the non-modified polynucleotide, but results in a conservative amino acid substitution or semi-conservative substitution, or a combination thereof in the expressed polypeptide.

### Polypeptides

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest,” “polypeptide,” “protein,” or “protein of interest” includes DesF and DesG polypeptides or other polypeptides (including variant polypeptides) described herein.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar Variants will be sufficiently similar to the amino acid sequence of the polypeptides described herein. Such variants generally retain the functional activity of the polypeptides described herein. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

An aspect provides an isolated polypeptide comprising a sequence set forth in SEQ ID NO:2 or SEQ ID NO:4. Another aspect provides an isolated polypeptide comprising a sequence set forth in SEQ ID NO:2 or SEQ ID NO:4, which contains 1, 2, 3, 4, 5, 6, 7, 8, 9, 10, 11, 12, 13, 14, 15, 16, 17, 18, 19, 20 or more amino acid substitution modifications relative to SEQ ID NO:2 or SEQ ID NO:4. Another aspect provides an isolated polypeptide comprising a sequence set forth in SEQ ID NO:2 or SEQ ID NO:4, which contains 20, 19, 18, 17, 16, 15, 14, 13, 12, 11, 10, 9, 8, 7, 6, 5, 4, 3, 2, or less amino acid substitution modifications relative to SEQ ID NO:2 or SEQ ID NO:4. In an aspect, amino acid substitutions to SEQ ID NO:2 do not occur at SER144, TYR157, or LYS161.

The substitution modifications can comprise conservative amino acid substitution modifications or semi-conservative substitution modification, or combinations thereof. A polypeptide can comprise less than 500, 400, 300, 290, 280, 270, 260, or 250 amino acids. In an aspect, one or more amino acids in any of SEQ ID NO:2 or SEQ ID NO:4 are substituted with another amino acid(s), the charge and polarity of which is similar to that of the original amino acid, i.e., a conservative amino acid substitution or semi-conservative substitution modification, or combinations thereof). Substitutes for an amino acid within the SEQ ID NO:2 or SEQ ID NO:4 can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these groups include: (1) acidic (anionic, negatively charged) amino acids such as aspartic acid and glutamic acid; (2) basic (cationic, positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within polypeptide sequences can be made by substituting one amino acid within one of these 4 groups with another amino acid within the same group. Biologically functional equivalents of modified SEQ ID NO:2 or SEQ ID NO:4 can have 50, 40, 20, 15, 10, 7, 5, 4, 3, 2, or fewer conservative amino acid changes. The encoding polynucleotide sequence (e.g., gene, plasmid DNA, cDNA, or synthetic DNA) will thus have corresponding base substitutions, permitting it to encode biologically functional equivalent forms of the modified SEQ ID NO:2 or SEQ ID NO:4 polypeptides.

In an aspect semi-conservative substitutions can be made in modified SEQ ID NO:2 or SEQ ID NO:4 including: (i) the substitution of a neutral polar amino acid residue with a neutral nonpolar (hydrophobic) amino acid residue; or (ii) the substitution of a neutral nonpolar (hydrophobic) amino acid residue with a neutral polar amino acid residue are also provided. In particular, semi-conservative substitutions of a neutral polar tyrosine residue with a hydrophobic amino acid residue are provided. Biologically functional equivalents of SEQ ID NO:2 or SEQ ID NO:4 polypeptides can have 50, 40, 20, 15, 10, 7, 5, 4, 3, 2, or fewer semi conservative amino acid changes. Nucleic acid molecules encoding any of the aforementioned modified defensin or defensin-like peptides are also provided herein. Recombinant polynucleotides comprising the aforementioned polynucleotides are also provided herein and in particular recombinant DNA molecules comprising a heterologous promoter that are operably linked to the aforementioned polynucleotides are also provided herein. In an aspect, the promoter is not naturally associated with the polynucleotide in nature. In an aspect, the promoter provides for higher or lower expression or different expression as compared to the wild type expression of the polynucleotide. Promoters are described in, for example, Sganzerla Martinez et al., CDBProm: the Comprehensive Directory of Bacterial Promoters, NAR Genomics and Bioinformatics, Volume 6, Issue 1, March 2024, Iqae018,

An aspect provides an isolated polypeptide comprising a sequence set forth in SEQ ID NO:2 or SEQ ID NO:4 and an indicator reagent, a detectable label, an amino acid spacer or an amino acid linker, a signal sequence (e.g., see signal sequence, Tat signal peptide, lipoprotein signal peptide, bacterial perpilini IV signal peptide), a stop transfer sequence, a transmembrane systems, proteins or domains (e.g., outer membrane proteins (OMPs) such as OmpA, OmpC, or the autotransporter protein AIDA-I, Lpp-OmpA system, which fuses the target protein with lipoprotein (Lpp) and OmpA, Sortase A enzyme system. a-agglutinin mating protein complex, which includes Aga1 and Aga2 subunits, flocculating proteins like Flo1p, Pir proteins, a transmembrane domain of the human platelet-derived growth factor receptor (PDGFR), and glycosylphosphatidylinositol (GPI) anchors a protein purification ligand, an affinity purification tag, a heterologous polypeptide, or a combination thereof. In an aspect, a heterologous polypeptide is not associated with the isolated polypeptide in nature. In an aspect, a heterologous polypeptide provides a beneficial property such as improved detection, purification, or expression.

Protein purification ligands and affinity purification tags can be, for example, Albumin-binding protein (ABP); Alkaline Phosphatase (AP); AU1 epitope; AUS epitope; Bacteriophage T7 epitope (T7-tag); Bacteriophage V5 epitope (V5-tag); Biotin-carboxy carrier protein (BCCP); Bluetongue virus tag (B-tag); Calmodulin binding peptide (CBP) Chloramphenicol Acetyl Transferase (CAT); Cellulose binding domain (CBP); Chitin binding domain (CBD); Choline-binding domain (CBD); Dihydrofolate reductase (DHFR); E2 epitope; FLAG epitope; Galactose-binding protein (GBP); Green fluorescent protein (GFP); Glu-Glu (EE-tag); Glutathione S-transferase (GST); Human influenza hemagglutinin (HA); HaloTag®; Histidine affinity tag (HAT); Horseradish Peroxidase (HRP); HSV epitope; Ketosteroid isomerase (KSI); KT3 epitope; LacZ; Luciferase; Maltose-binding protein (MBP); Myc epitope; NusA; PDZ domain; PDZ Iigand; Polyarginine (Arg-tag); Polyaspartate (Asp-tag); Polycysteine (Cys-tag); Polyhistidine (His-tag); Polyphenylalanine (Phe-tag); Profinity eXact; Protein C; S1-tag; S-tag; Streptavadin-binding peptide (SBP); Staphylococcal protein A (Protein A); Staphylococcal protein G (Protein G); Strep-tag; Streptavadin; Small Ubiquitin-like Modifier (SUMO); Tandem Affinity Purification (TAP); T7 epitope; Thioredoxin (Trx); TrpE; Ubiquitin; Universal; VSV-G.

An amino acid spacer or amino acid linker is a short amino acid sequence (e.g., about 5, 10, 15, 20 or more amino acids) that can be used to, e.g., connect polypeptide molecules. Spacers and linkers can have different levels of flexibility and have several uses. Amino acid spacers and linkers can be used to separate domains in proteins to prevent unwanted interactions between domains in a single protein; to connect molecules such as a fluorophore to a polypeptide; to separate peptides from labels and dyes; to modify the natural hydropathy of a polypeptide (e.g., aminohexanoic acid (Ahx) as a hydrophobic spacer and polyethylene glycol (PEG) as a hydrophilic spacer); and to improve folding and stability of a polypeptide (e.g. GS linker).

An aspect provides a fusion protein comprising a sequence set forth in SEQ ID NO:2 or SEQ ID NO:4 and a heterologous polypeptide.

Polypeptides and polynucleotides that are sufficiently similar to polypeptides and polynucleotides described herein (e.g., desF and desG) can be used herein. Polypeptides and polynucleotides that about 85, 90, 91, 92, 93, 94 95, 96, 97, 98, 99 99.5% or more homology or identity to polypeptides and polynucleotides described herein (e.g., desF and desG and variants thereof) can also be used herein.

### Constructs and Cassettes

A recombinant construct is a polynucleotide having heterologous polynucleotide elements. Recombinant constructs include expression cassettes or expression constructs, which refer to an assembly that is capable of directing the expression of a polynucleotide or gene of interest. An expression cassette generally includes regulatory elements such as a promoter that is operably linked to (so as to direct transcription of) a polynucleotide and often includes a polyadenylation sequence as well.

An expression cassette can comprise to a fragment of DNA comprising a coding sequence of a selected gene (e.g. desF and desG) and regulatory elements preceding (5′ non-coding sequences) and following (3′ non-coding sequences) the coding sequence that are required for expression of the selected gene product. Thus, an expression cassette can comprise 1) a promoter sequence; 2) one or more coding sequences [“ORF” ]; and, optionally, 3) a 3′ untranslated region (i.e., an intrinsic terminator or rho-dependent terminator). The expression cassette is usually included within a vector, to facilitate cloning and transformation. Different expression cassettes can be transformed into different organisms including bacteria, yeast, plants, and mammalian cells, as long as the correct regulatory elements are used for each host.

A recombinant construct or expression cassette can be contained within a vector. In addition to the components of the recombinant construct, the vector can include, for example, one or more selectable markers, a signal which allows the vector to exist as single-stranded DNA (e.g., a M13 origin of replication), one or more multiple cloning sites, and an origin of replication (e.g., a SV40 or adenovirus origin of replication).

Generally, a polynucleotide or gene that is introduced into a genetically engineered organism is part of a recombinant construct. A polynucleotide can comprise a gene of interest, e.g., a coding sequence for a protein, or can be a sequence that is capable of regulating expression of a gene, such as a regulatory element, an antisense sequence, a sense suppression sequence, or a miRNA sequence. A recombinant construct can include, for example, regulatory elements operably linked 5′ or 3′ to a polynucleotide encoding one or more polypeptides of interest. For example, a promoter can be operably linked with a polynucleotide encoding one or more polypeptides of interest when it is capable of affecting the expression of the polynucleotide (i.e., the polynucleotide is under the transcriptional control of the promoter). Polynucleotides can be operably linked to regulatory elements in sense or antisense orientation. The expression cassettes or recombinant constructs can additionally contain a 5′ leader polynucleotide. A leader polynucleotide can contain a promoter as well as an upstream region of a gene. The regulatory elements (i.e., promoters, enhancers, transcriptional regulatory regions, translational regulatory regions, and translational termination regions) and/or the polynucleotide encoding a signal anchor can be native/analogous to the host cell or to each other. Alternatively, the regulatory elements can be heterologous to the host cell or to each other. See, U.S. Pat. No. 7,205,453 and U.S. Patent Application Publication Nos. 2006/0218670 and 2006/0248616. The expression cassette or recombinant construct can additionally contain one or more selectable marker genes. A polynucleotide can be operably linked when it is positioned adjacent to or close to one or more regulatory elements, which direct transcription and/or translation of the polynucleotide.

A promoter is a nucleotide sequence that is capable of controlling the expression of a coding sequence or gene. Promoters are generally located 5′ of the sequence that they regulate. Promoters can be derived in their entirety from a native gene, or be composed of different elements derived from promoters found in nature, and/or comprise synthetic nucleotide segments. Those skilled in the art will readily ascertain that different promoters can regulate expression of a coding sequence or gene in response to a particular stimulus, e.g., in a cell- or tissue-specific manner, in response to different environmental or physiological conditions, or in response to specific compounds. Promoters are typically classified into two classes: inducible and constitutive. A constitutive promoter refers to a promoter that allows for continual transcription of the coding sequence or gene under its control.

An inducible promoter refers to a promoter that initiates increased levels of transcription of the coding sequence or gene under its control in response to a stimulus or an exogenous environmental condition. If inducible, there are inducer polynucleotides present therein that mediate regulation of expression so that the associated polynucleotide is transcribed only when an inducer molecule is present. A directly inducible promoter refers to a regulatory region, wherein the regulatory region is operably linked to a gene encoding a protein or polypeptide, where, in the presence of an inducer of the regulatory region, the protein or polypeptide is expressed. An indirectly inducible promoter refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by inducible promoter.

A promoter can be any polynucleotide that shows transcriptional activity in the chosen host microorganism. A promoter can be naturally-occurring, can be composed of portions of various naturally-occurring promoters, or may be partially or totally synthetic. Guidance for the design of promoters is derived from studies of promoter structure, such as that of Harley and Reynolds, Nucleic Acids Res., 15, 2343-61 (1987). In addition, the location of the promoter relative to the transcription start can be optimized. Many suitable promoters for use in microorganisms and yeast are well known in the art, as are polynucleotides that enhance expression of an associated expressible polynucleotide.

A selectable marker can provide a means to identify microorganisms that express a desired product. Selectable markers include, but are not limited to, ampicillin resistance for prokaryotes such as E. coli, neomycin phosphotransferase, which confers resistance to the aminoglycosides neomycin, kanamycin and paromycin (Herrera-Estrella, EMBO J. 2:987-995, (1983)); dihydrofolate reductase, which confers resistance to methotrexate (Reiss, Plant Physiol. (Life Sci. Adv.) 13:143-149, (1994)); trpB, which allows cells to utilize indole in place of tryptophan; hisD, which allows cells to utilize histinol in place of histidine (Hartman, Proc. Natl. Acad. Sci., USA 85:8047, (1988)); mannose-6-phosphate isomerase which allows cells to utilize mannose (WO 94/20627); hygro, which confers resistance to hygromycin (Marsh, Gene 32:481-485, (1984)); ornithine decarboxylase, which confers resistance to the ornithine decarboxylase inhibitor, 2-(difluoromethyl)-DL-ornithine (DFMO; McConlogue, In: Current Communications in Molecular Biology, Cold Spring Harbor Laboratory ed., (1987)); deaminase from Aspergillus terreus, which confers resistance to Blasticidin S (Tamura, Biosci. Biotechnol. Biochem. 59:2336-2338, (1995)); phosphinothricin acetyltransferase gene, which confers resistance to phosphinothricin (White et al., Nucl. Acids Res. 18:1062, (1990); Spencer et al., Theor. Appl. Genet. 79:625-633, (1990)); a mutant acetolactate synthase, which confers imidazolione or sulfonylurea resistance (Lee et al., EMBO J. 7:1241-1248, (1988)), a mutant EPSPV-synthase, which confers glyphosate resistance (Hinchee et al., BioTechnology 91:915-922, (1998)); a mutant psbA, which confers resistance to atrazine (Smeda et al., Plant Physiol. 103:911-917, (1993)), a mutant protoporphyrinogen oxidase (see U.S. Pat. No. 5,767,373), or other markers conferring resistance to an herbicide such as glufosinate.

A transcription termination region of a recombinant construct or expression cassette is a downstream regulatory region including a stop codon and a transcription terminator sequence. Transcription termination regions that can be used can be homologous to the transcriptional initiation region, can be homologous to the polynucleotide encoding a polypeptide of interest, or can be heterologous (i.e., derived from another source). A transcription termination region or can be naturally occurring, or wholly or partially synthetic. 3′ non-coding sequences encoding transcription termination regions may be provided in a recombinant construct or expression construct and may be from the 3′ region of the gene from which the initiation region was obtained or from a different gene. A large number of termination regions are known and function satisfactorily in a variety of hosts when utilized in both the same and different genera and species from which they were derived. Termination regions can also be derived from various genes native to the preferred hosts. The termination region is usually selected more for convenience rather than for any particular property.

An expression cassette can comprise a polynucleotide as set forth in SEQ ID NO:1, SEQ ID NO:3, or both. An expression cassette can comprise a first polynucleotide as set forth in SEQ ID NO:1, wherein the first polynucleotide optionally comprises less than 770 nucleic acids (e.g., about less than 900, 850, 800, 790, 780, 775, 770, 768, 765, or 760 nucleic acids); and a second polynucleotide comprising at least one expression control sequence.

Polynucleotides can be operably linked to one or more expression control sequences. An expression control sequence can be a heterologous sequence. For example, one or more expression control sequences can be incorporated into an expression construct or vector so that expression control sequences effectively control expression of a polynucleotide. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. The expression control sequence can be a promoter (e.g., a heterologous promoter) operably linked to the first polynucleotide.

An expression cassette can comprise a first polynucleotide as set forth in SEQ ID NO:3, wherein the first polynucleotide optionally comprises less than 770 nucleic acids (e.g., about less than 900, 850, 800, 790, 780, 775, 770, 768, 765, or 760 nucleic acids); and a second polynucleotide comprising at least one expression control sequence. The expression control sequence can be a promoter (e.g., a heterologous promoter) operably linked to the first polynucleotide.

The procedures described herein employ, unless otherwise indicated, conventional techniques of chemistry, molecular biology, microbiology, recombinant DNA, genetics, immunology, cell biology, cell culture and transgenic biology, which are within the skill of the art. (See, e.g., Maniatis, et al., Molecular Cloning, Cold Spring Harbor Laboratory Press, Cold Spring Harbor, N.Y. (1982); Sambrook et al., (1989); Sambrook and Russell, Molecular Cloning, 3rd Ed., Cold Spring Harbor Laboratory Press, Cold Spring Harbor, N.Y. (2001); Ausubel, et al., Current Protocols in Molecular Biology, John Wiley & Sons (including periodic updates) (1992); Glover, DNA Cloning, IRL Press, Oxford (1985); Russell, Molecular biology of plants: a laboratory course manual, Cold Spring Harbor Laboratory Press, Cold Spring Harbor, N.Y. (1984); Anand, Techniques for the Analysis of Complex Genomes, Academic Press, NY (1992); Guthrie and Fink, Guide to Yeast Genetics and Molecular Biology, Academic Press, NY (1991); Harlow and Lane, Antibodies, Cold Spring Harbor Laboratory Press, Cold Spring Harbor, N.Y. (1988); Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, A. R. Liss, Inc. (1987); Immobilized Cells And Enzymes, IRL Press (1986); B. Perbal, A Practical Guide To Molecular Cloning (1984); the treatise, Methods In Enzymology, Academic Press, Inc., NY); Methods In Enzymology, Vols. 154 and 155, Wu, et al., eds.; Immunochemical Methods In Cell And Molecular Biology, Mayer and Walker, eds., Academic Press, London (1987); Handbook Of Experimental Immunology, Volumes I-IV, D. M. Weir and C. C. Blackwell, eds. (1986); Riott, Essential Immunology, 6th Edition, Blackwell Scientific Publications, Oxford (1988); Fire, et al., RNA Interference Technology From Basic Science to Drug Development, Cambridge University Press, Cambridge (2005); Schepers, RNA Interference in Practice, Wiley-VCH (2005); Engelke, RNA Interference (RNAi): The Nuts & Bolts of siRNA Technology, DNA Press (2003); Gott, RNA Interference, Editing, and Modification: Methods and Protocols (Methods in Molecular Biology), Human Press, Totowa, N.J. (2004); and Sohail, Gene Silencing by RNA Interference: Technology and Application, CRC (2004)).

### Vectors

Vectors for stable transformation of microorganisms can be obtained from commercial vendors or constructed from publicly available sequence information. Expression vectors can be engineered to produce heterologous and/or homologous protein(s) of interest (e.g., desF and desG). Such vectors are useful for recombinantly producing a protein of interest and for modifying the natural phenotype of host cells.

If desired, polynucleotides can be cloned into a vector comprising, for example, expression control elements, including for example, origins of replication, promoters, enhancers, or other regulatory elements that drive expression of the polynucleotides in host cells. A vector can be, for example, extrachromosomal (e.g., episome) or integrating (for being incorporated into the host chromosomes), autonomously replicating or not, multi or low copy, double-stranded or single-stranded, naked or complexed with other molecules (e.g., vectors complexed with lipids or polymers to form particulate structures such as liposomes, lipoplexes or nanoparticles, vectors packaged in a viral capsid, and vectors immobilized onto solid phase particles, etc.).

A vector can be a non-viral vector (e.g., a plasmid, cosmid, artificial chromosome and the like) or a viral vector. In an aspect, a vector is an adenoviral vector. In some aspects, the vector can be an adeno-associated viral (AAV) vector from any serotype such as AAV1, AAV2, AAV3, AAV4, AAV5, AAV6, AAV7, AAV8, AAV9, AAV10, AAV11, AAV12, and variants thereof.

To confirm the presence of recombinant polynucleotides or recombinant genes in transgenic cells, a polymerase chain reaction (PCR) amplification or Southern blot analysis can be performed. Expression products of the recombinant polynucleotides or recombinant genes can be detected in any of a variety of ways, and include for example, western blot and enzyme assay. Once recombinant organisms have been obtained, they can be grown in cell culture.

A vector can comprise a polynucleotide as set forth in SEQ ID NO:1 or SEQ ID NO:3 or an expression cassette comprising SEQ ID NO:1 or SEQ ID NO:3.

### Recombinant Microorganisms

A recombinant, transgenic, or genetically engineered microorganism is a microorganism, e.g., bacteria that has been genetically modified from its native state. Thus, a “recombinant bacterium” or “recombinant bacterial cell” refers to a bacterial cell that has been genetically modified from the native state. A recombinant bacterial cell can have, for example, nucleotide insertions, nucleotide deletions, nucleotide substitutions nucleotide rearrangements, gene disruptions, recombinant polynucleotides, heterologous polynucleotides, deleted polynucleotides, nucleotide modifications, or combinations thereof introduced into its DNA. These genetic modifications can be present in the chromosome of the bacterial cell, or on a plasmid in the bacterial cell. Recombinant cells disclosed herein can comprise exogenous polynucleotides on plasmids. Alternatively, recombinant cells can comprise exogenous polynucleotides stably incorporated into their chromosome.

A heterologous or exogenous polypeptide or polynucleotide refers to any polynucleotide or polypeptide that does not naturally occur or that is not present in the starting target microorganism. For example, a polynucleotide that is transformed into a bacterial cell that does naturally or otherwise comprise the bacterial polynucleotide is a heterologous or exogenous polynucleotide. A heterologous or exogenous polypeptide or polynucleotide can be a wild-type, synthetic, or mutated polypeptide or polynucleotide. In an aspect, a heterologous or exogenous polypeptide or polynucleotide is not naturally present in a starting target microorganism and is from a different genus or species than the starting target microorganism.

A homologous or endogenous polypeptide or polynucleotide refers to any polynucleotide or polypeptide that naturally occurs or that is otherwise present in a starting target microorganism. For example, a polynucleotide that is naturally present in a bacterial cell is a homologous or endogenous polynucleotide. In an aspect, a homologous or endogenous polypeptide or polynucleotide is naturally present in a starting target microorganism.

A recombinant microorganism can comprise one or more polynucleotides not present in a corresponding wild-type cell, wherein the polynucleotides have been introduced into that microorganism using recombinant DNA techniques, or which polynucleotides are not present in a wild-type microorganism and is the result of one or more mutations. A genetically modified or recombinant microorganism can be, for example, a bacterium.

An aspect provides a recombinant cell comprising a polynucleotide as set forth in SEQ ID NO:1 or SEQ ID NO:3. The polynucleotide as set forth in SEQ ID NO:1 or SEQ ID NO:3 can be less than about 900, 850, 800, 790, 780, 775, 770, 768, 765, or 760 nucleic acids nucleic acids in length. A recombinant cell can be, for example, a bacterial cell, a fungal cell, or a eukaryotic cell.

### Methods

Provided herein are methods of producing 17α-hydroxysteroid dehydrogenase (17α-HSDH) comprising culturing a recombinant cell comprising a polynucleotide as set forth in SEQ ID NO:1 in suitable cell culture media and recovering 17α-hydroxysteroid dehydrogenase. In an aspect, the 17α-HSDH polypeptide is as set forth in SEQ ID NO:2. The polynucleotide or polypeptide can be any 17α-HSDH polynucleotide or polypeptide as described herein. In an aspect, the 17α-HSDH can be recovered from the cell culture media or cells can be lysed and the 17α-HSDH collected.

Also provided herein are methods of producing 17β-hydroxysteroid dehydrogenase (17β-HSDH) comprising culturing a recombinant cell comprising a polynucleotide as set forth in SEQ ID NO:3 in suitable cell culture media and recovering 17β-hydroxysteroid dehydrogenase. In an aspect, the 17β-HSDH polypeptide is as set forth in SEQ ID NO:4. The polynucleotide or polypeptide can be any 17β-HSDH polynucleotide or polypeptide as described herein. In an aspect, the 17β-HSDH can be recovered from the cell culture media or cells can be lysed and the 17β-HSDH collected.

An aspect provides a method of producing epitestosterone comprising contacting a recombinant cell comprising a polynucleotide as set forth in SEQ ID NO:1 or a cell expressing SEQ ID NO:2 in suitable cell culture media and recovering and recovering epitestosterone. The polynucleotide can be any 17α-HSDH polynucleotide as described herein. In an aspect, NADPH is added to the cell culture media. In an aspect, the epitestosterone can be recovered from the cell culture media or cells can be lysed and the epitestosterone collected. The recombinant cell can express androstenedione naturally or recombinantly.

An aspect provides a method of producing testosterone comprising contacting a recombinant cell comprising a polynucleotide as set forth in SEQ ID NO:3 or a cell expressing SEQ ID NO:4 in suitable cell culture media and recovering and recovering testosterone. The polynucleotide can be any 17β-HSDH polynucleotide as described herein. In an aspect, the testosterone can be recovered from the cell culture media or cells can be lysed and the testosterone collected. The recombinant cell can express androstenedione naturally or recombinantly.

Also provided herein are methods of identifying prostate cancer, resistant prostate cancer (prostate cancer that continues to grow even when testosterone levels are low), or advancing prostate cancer (where cancer has spread outside of the prostate) in a patient comprising detecting a level of 17α-hydroxysteroid dehydrogenase (17α-HSDH) present in a prostatectomy sample, a urine sample, or a fecal sample of the patient, wherein an elevated level of 17α-HSDH as compared to a control sample or standard indicates prostate cancer, resistant prostate cancer, or advancing prostate cancer.

Also provided are methods of identifying prostate cancer, resistant prostate cancer, or advancing prostate cancer in a patient comprising detecting a level of 17β-hydroxysteroid dehydrogenase (17β-HSDH) present in a prostatectomy sample, a urine sample, or a fecal sample of the patient, wherein an elevated level of 17β-HSDH as compared to a control sample or standard indicates prostate cancer, resistant prostate cancer, or advancing prostate cancer.

An elevated level of 17α-HSDH or 17β-HSDH can be detected using, for example, mass spectrometry or immunoassays. An elevated level of HSDH or 17β-HSDH can also be detected using PCR such as quantitative PCR methods.

A positive control or standard can be a sample containing a detectable amount of 17α-HSDH or 17β-HSDH. In an aspect, a positive control or standard can comprise 17α-HSDH or 17β-HSDH synthesized in vitro or otherwise obtained. A control or standard can indicate a cut off value, above which indicates a positive result. A negative control or standard can be a sample containing no detectable amount of 17α-HSDH or 17β-HSDH. For example, a negative control can include water or buffer.

In an aspect, where a patient is identified as having prostate cancer, the patient can further be treated for prostate cancer by prostatectomy, hormone therapy, active surveillance, radiation therapy, high-intensity focused ultrasound, cryotherapy, chemotherapy, immunotherapy, bisphosphonate therapy, or other suitable therapy.

In another aspect, a prostate cancer patient can be treated by administering one or more antibiotics effective against bacteria expressing desAB, desG, and/or desF and/or an inhibitor of desAB, desG, and/or desF. Examples of suitable antibiotics include penicillins (e.g., amoxicillin, ampicillin, nafcillin, and oxacillin), cephalosporins (e.g., cefalexin, cephalexin, cefotaxime, and ceftaroline), glycopeptides (e.g., vancomycin and teicoplanin), oxazolidinones (e.g. linezolid), lipoglycopeptides (e.g., dalbavancin and oritavancin), clindamycin, daptomycin, and tetracycline.

### Methods of Monitoring

In an aspect, the effects of treatment, advancement of prostate cancer, or remission of prostate cancer can be monitored. In an aspect, the detection of levels of 17α-HSDH and/or 17β-HSDH in a biological sample from the patient as compared to a control sample or control standard alone as described herein at 2, 3, 4, 5, 6 or more time intervals can be completed on a patient. The time intervals can be about 1, 5, 10, 14, 30, 60, 90, 120, 160, 200, 300, 360 or more days apart. In an aspect, one or more treatments can be administered between intervals. For example, prostate cancer can be detected according to any suitable method, e.g., by detecting 17α-HSDH and/or 17β-HSDH levels. One or more treatments can be administered and then levels of 17α-HSDH and/or 17β-HSDH can be detected after administration of the treatment (e.g., about 1, 5, 10, 14, 30, 60, 90, 120, 160, 200, 300, 360 or more days). The result can inform a doctor about the status of the patient (e.g., the patient is responding to treatment or not responding to treatment; or the patient is in remission). Decreasing levels of 17α-HSDH and/or 17 β-HSDH can indicate that the patient is responding to treatment.

In an aspect, a method of monitoring prostate cancer in a subject can comprise detecting at two or more time points: an amount of 17α-HSDH and/or 17β-HSDH; and comparing results at the two or more points such that the prostate cancer is monitored in the subject. In an example, assuming that a subject was initially diagnosed at time point 1 with prostate cancer, then at time point 2 a finding of a reduced level of 17α-HSDH and/or 17β-HSDH as compared to time point 1 in the subject; can indicate that the treatment is working or that the subject is in remission. No change would indicate that the treatment is not working and in some cases that the prostate cancer is not advancing.

In an example, assuming that a subject was initially diagnosed at time point 1 with prostate cancer, then at time point 2 a finding of a higher level of 17α-HSDH and/or 17β-HSDH as compared to time point 1 in the subject; can indicate that the prostate cancer is advancing. As discussed above, results at further time points can be determined and compared to the earlier time points.

In an aspect, epiT can be used in vitro to act as an AR agonist and drive proliferation of cancer cells such as prostate cancer cells. For example, about 5, 10, 15, or 20 nM epiT can be added to a cancer cell culture to induce proliferation of the cancer cells.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the aspects described in broad terms above.

### EXAMPLES

### Example 1: a Novel Pathway for the Formation of epiT by the Gut Microbiota

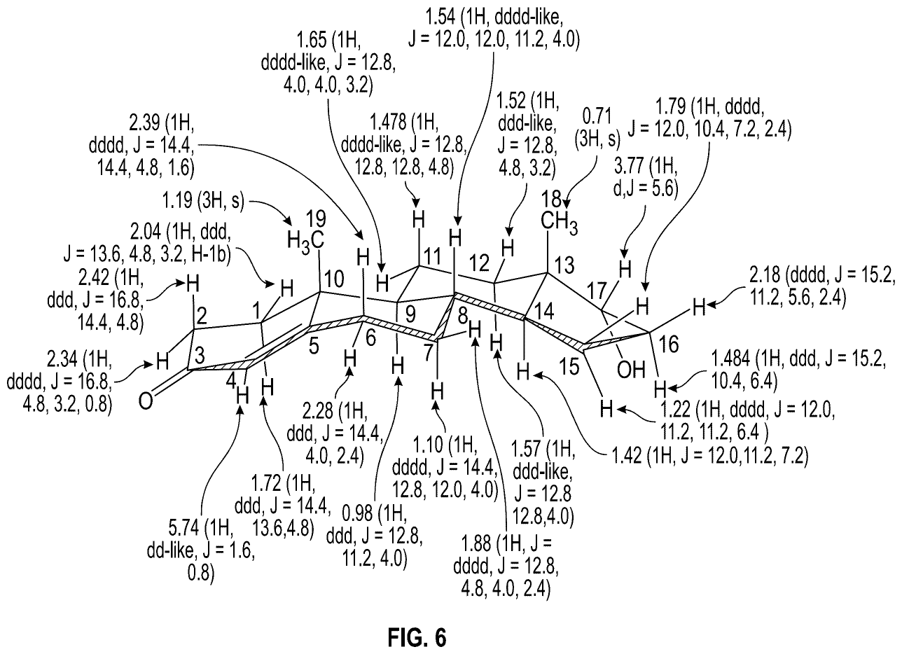

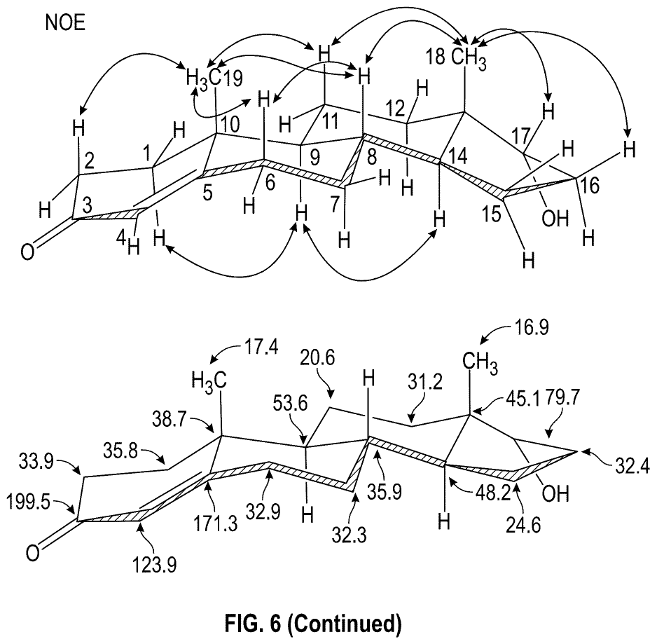

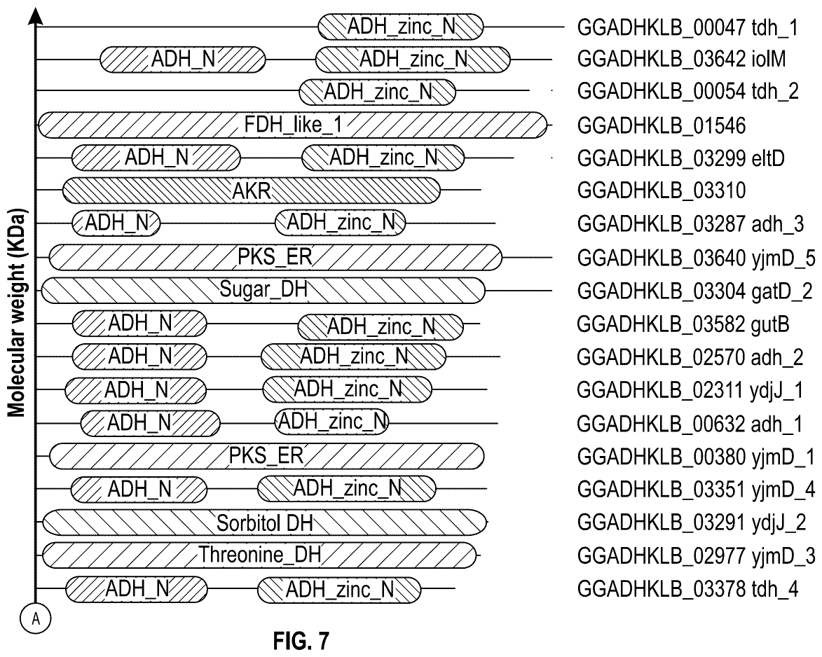

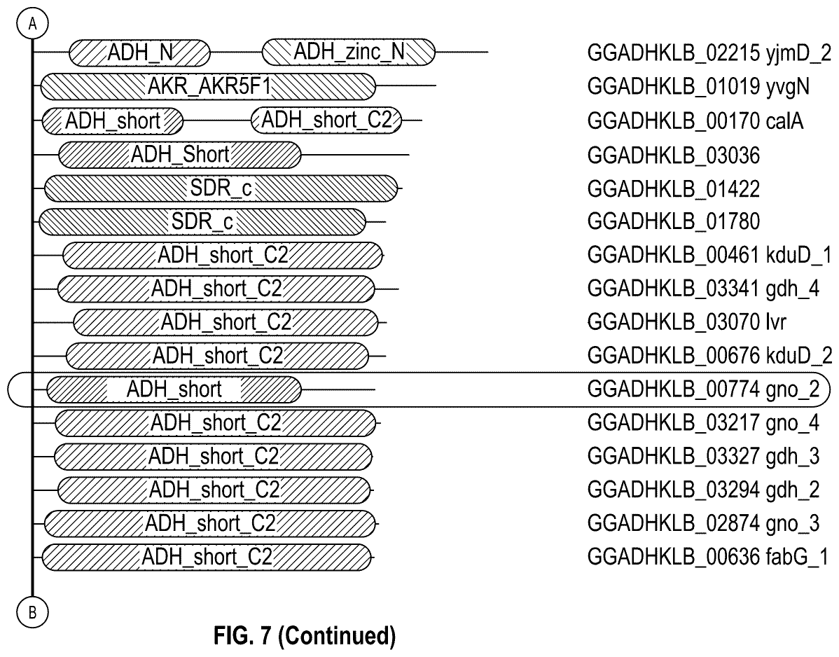

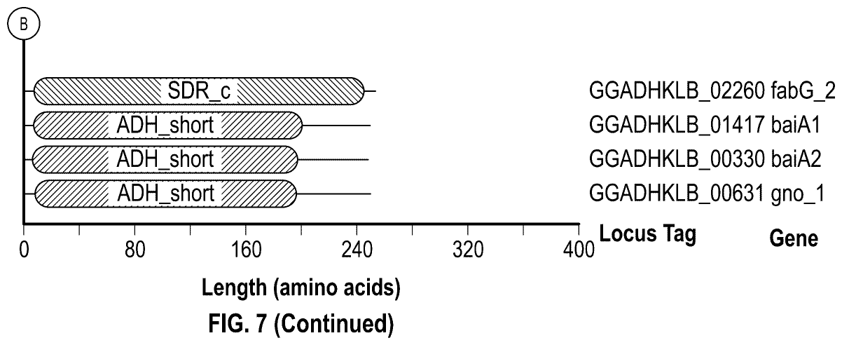

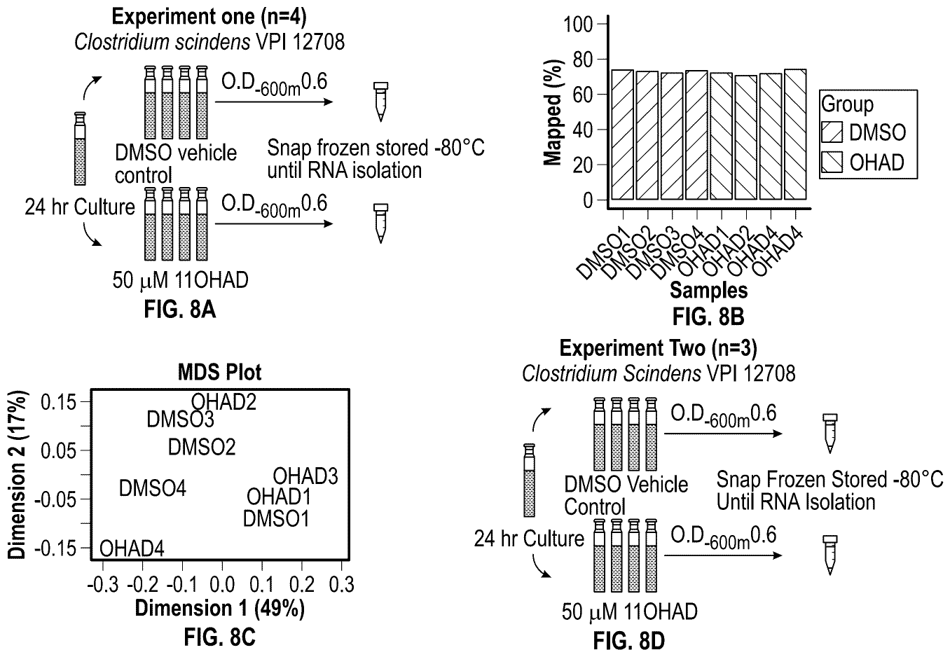

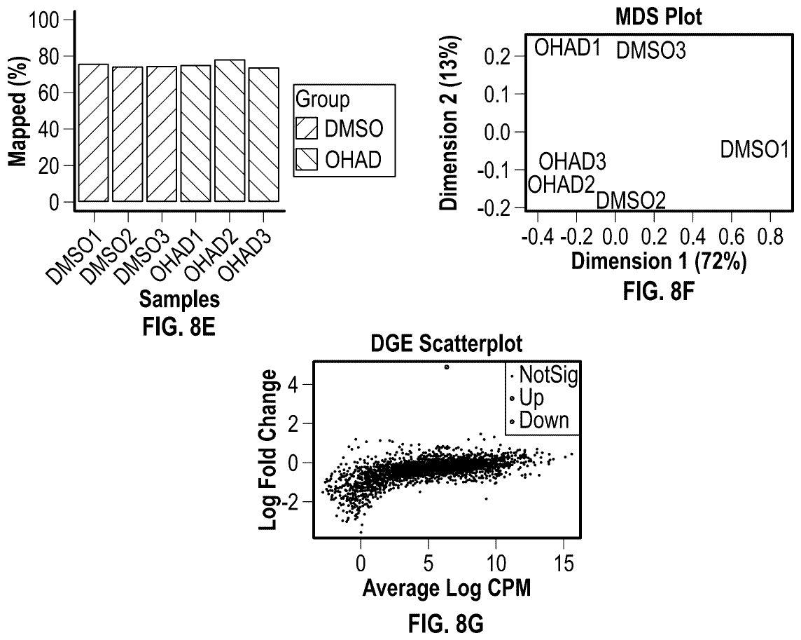

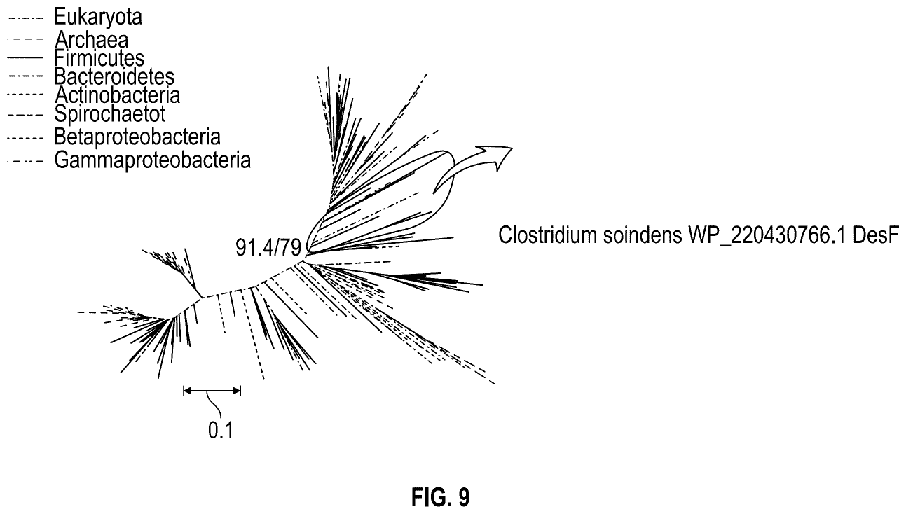

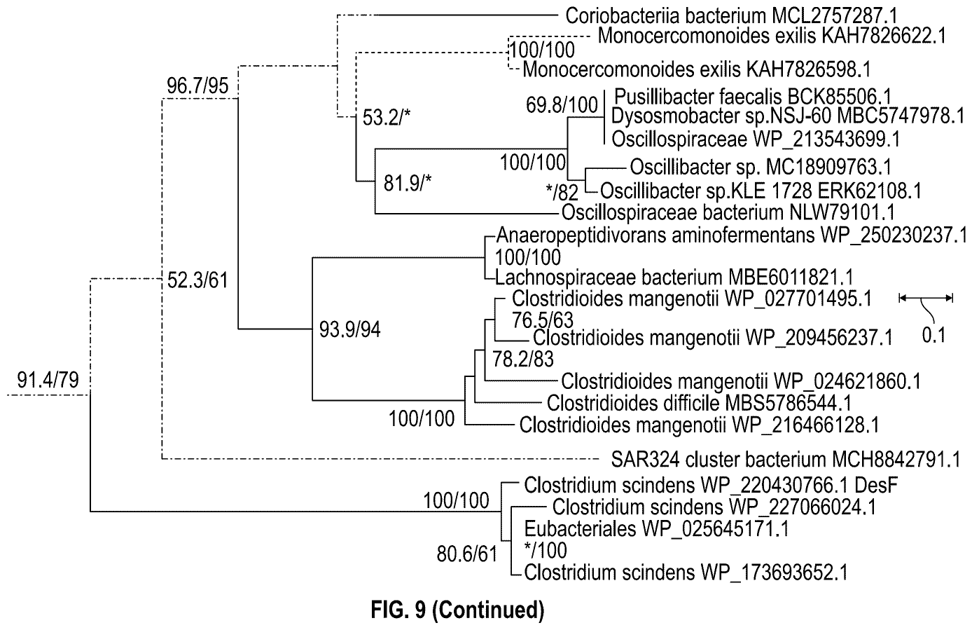

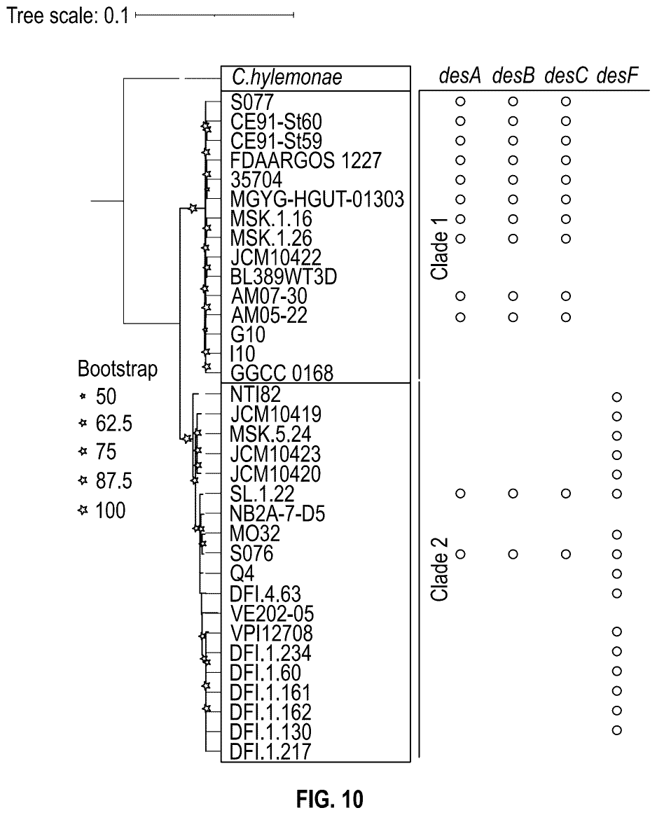

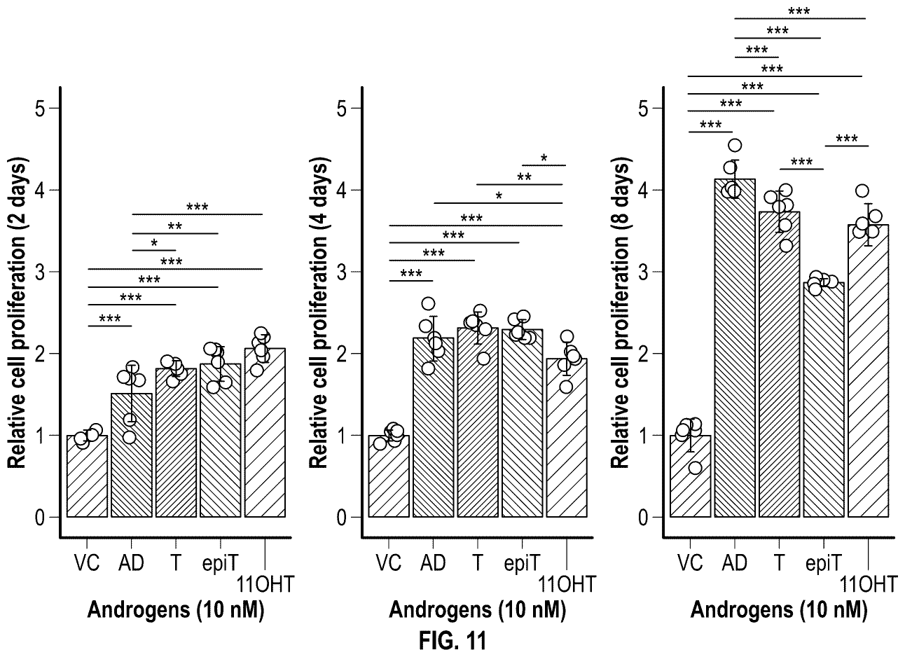

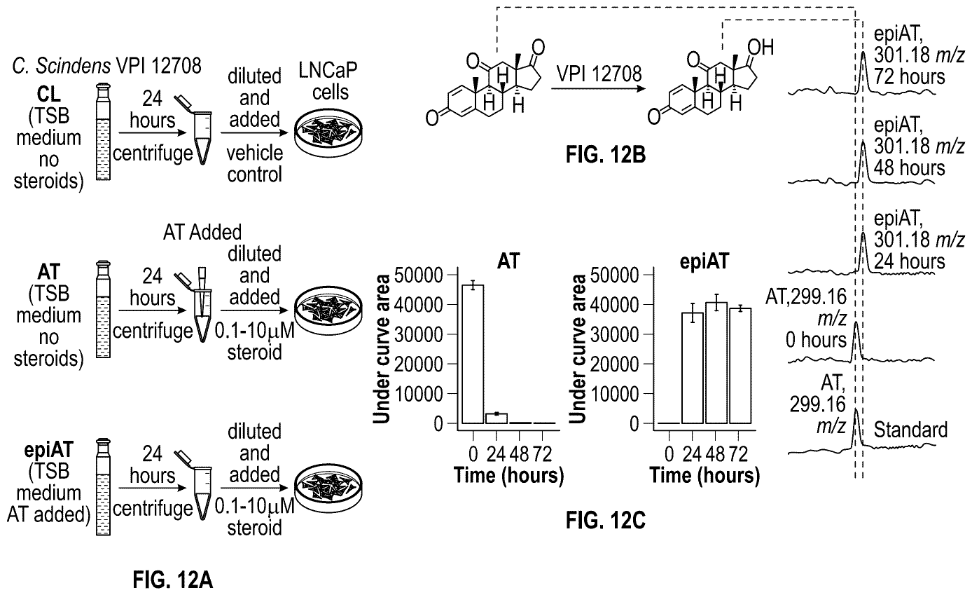

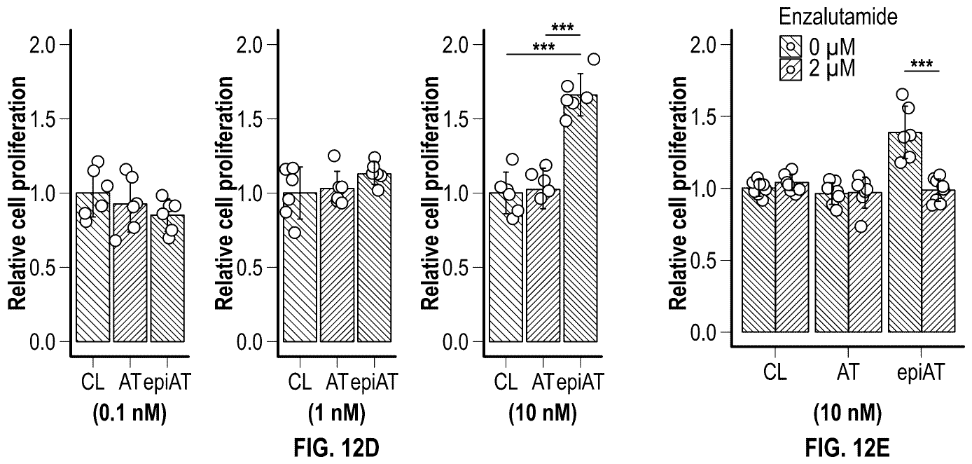

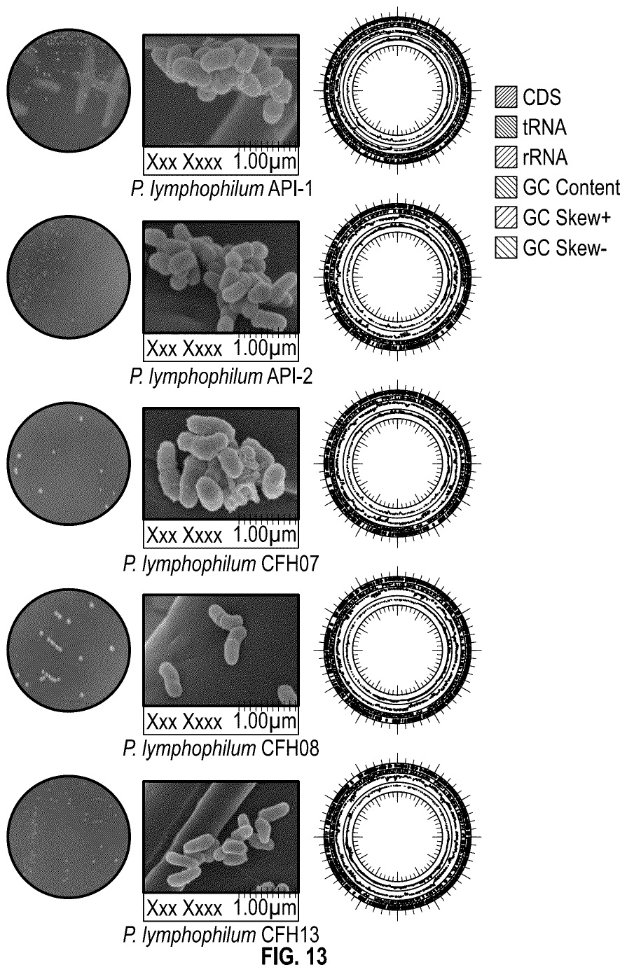

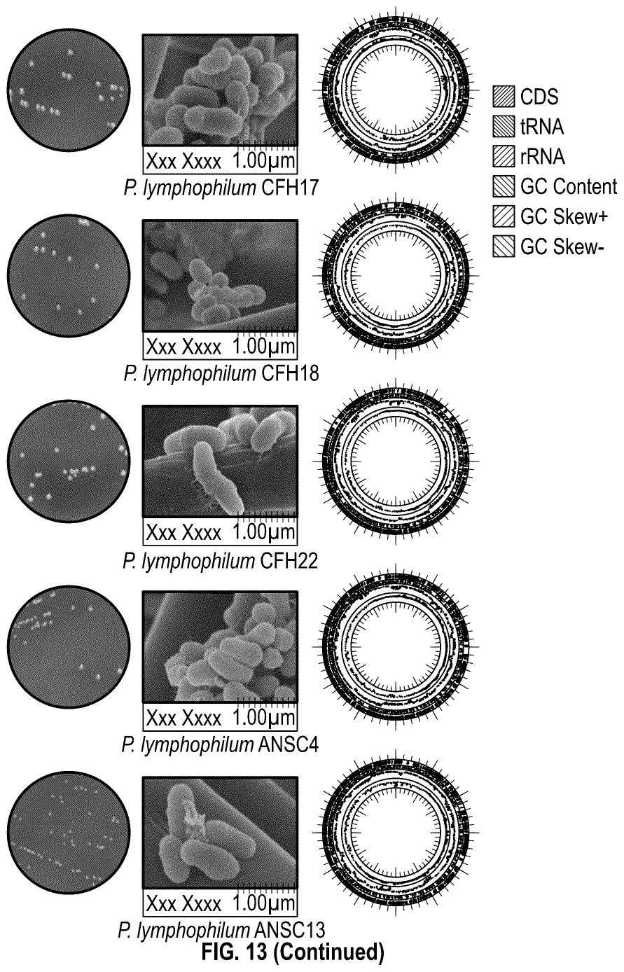

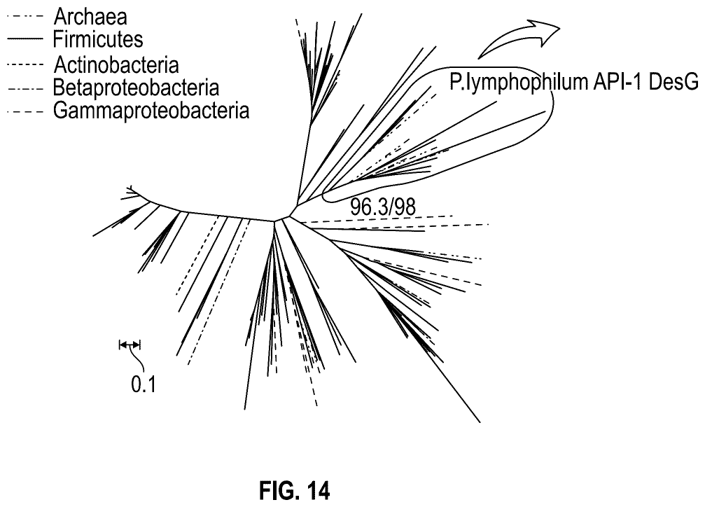

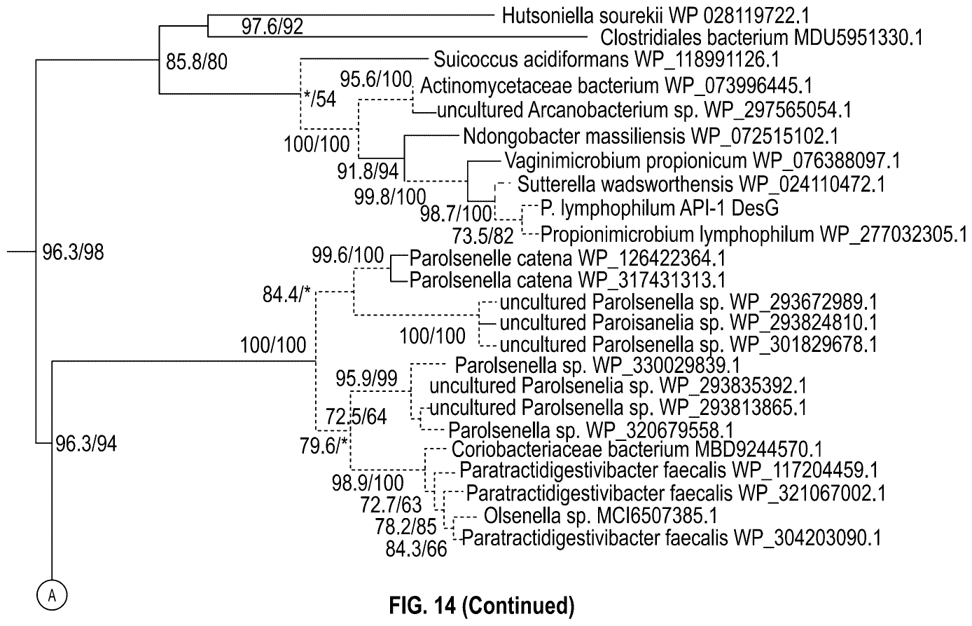

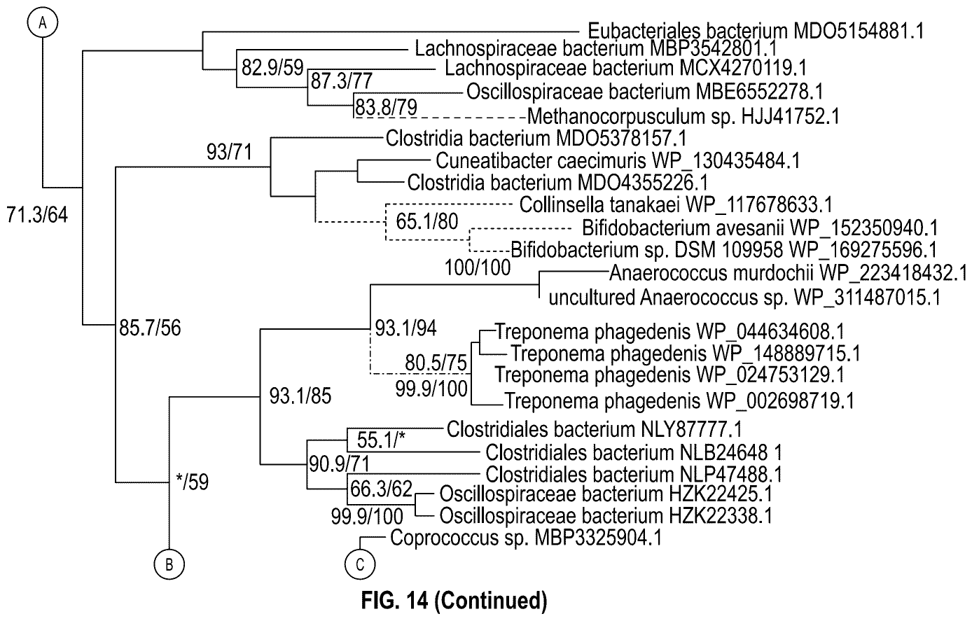

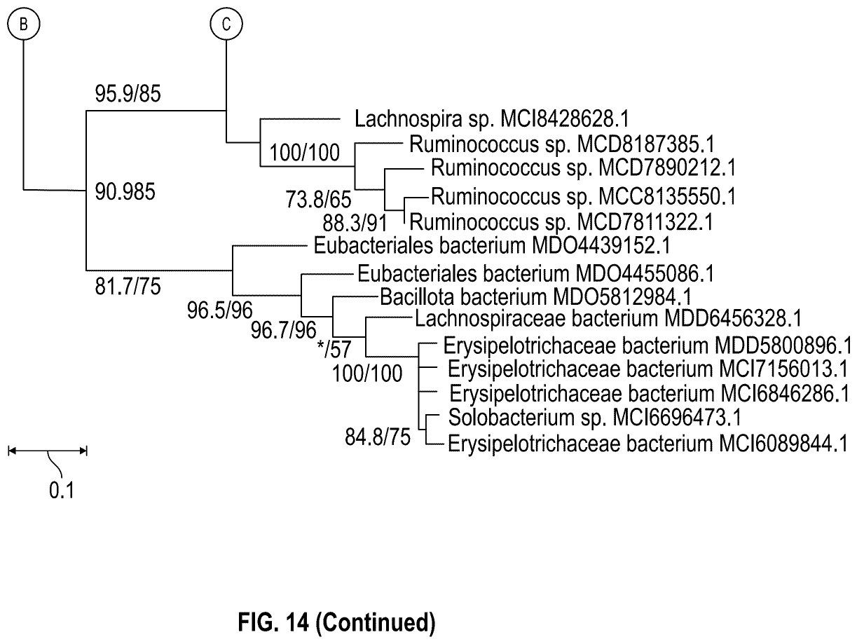

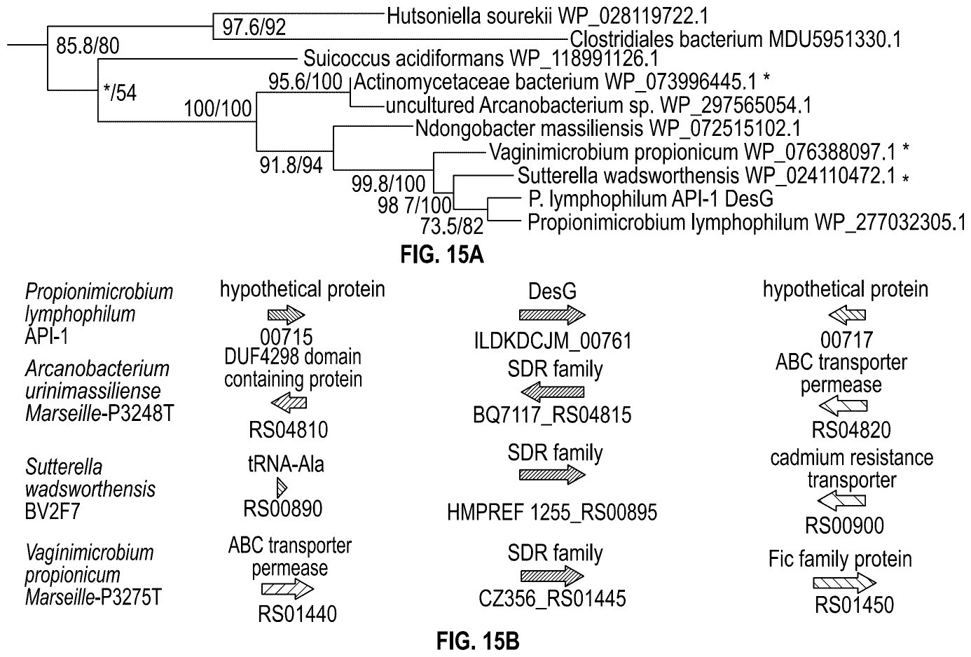

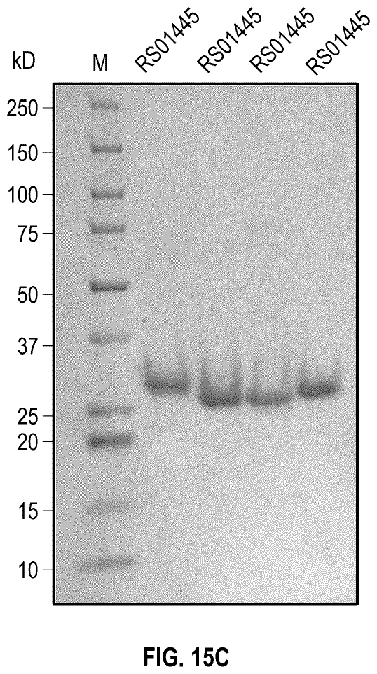

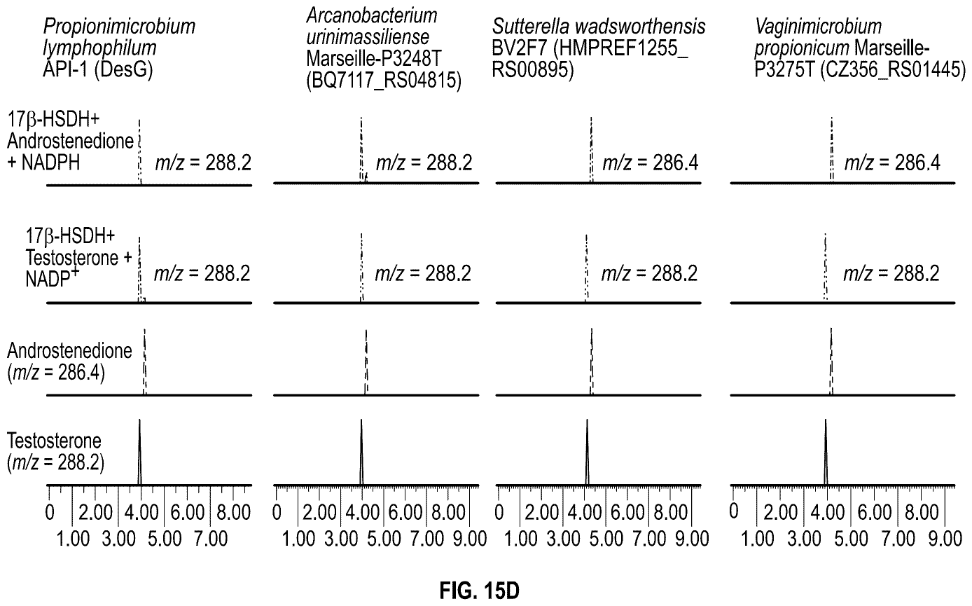

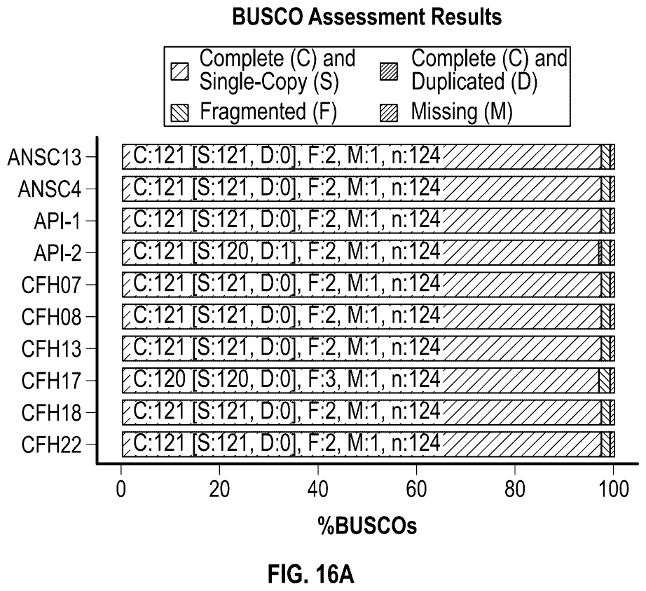

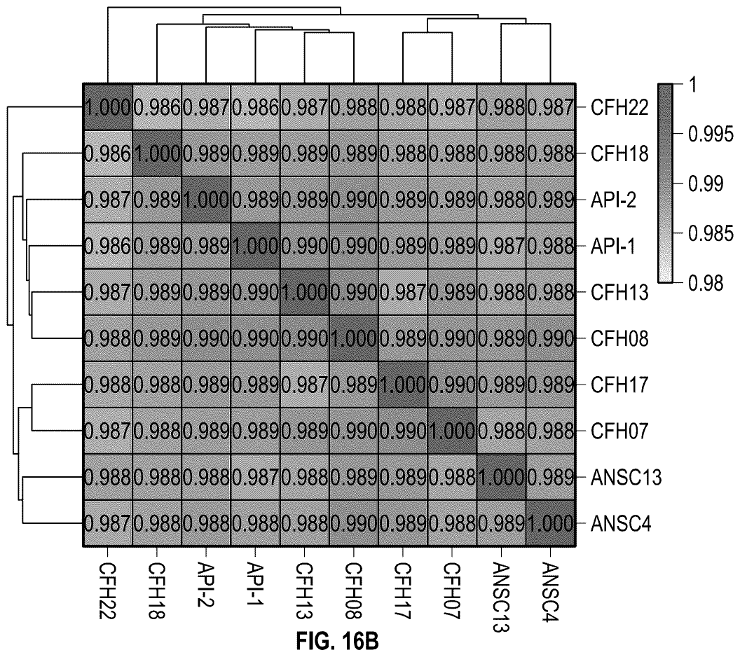

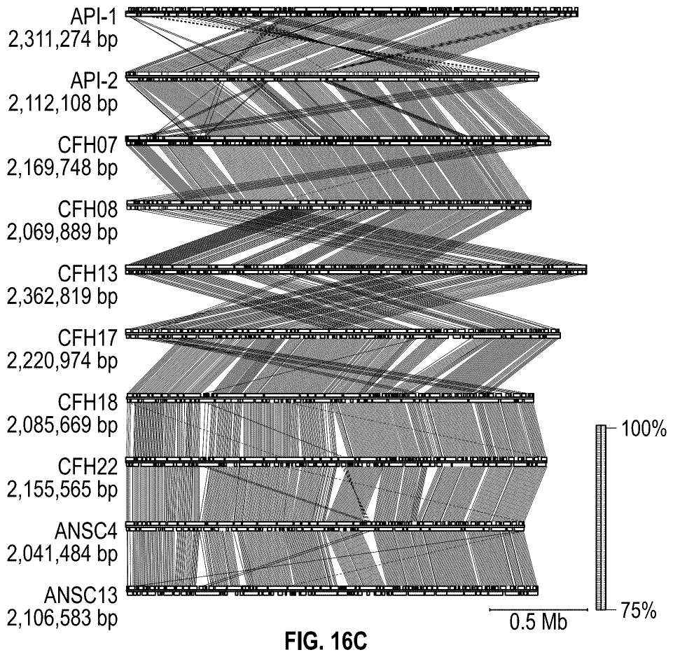

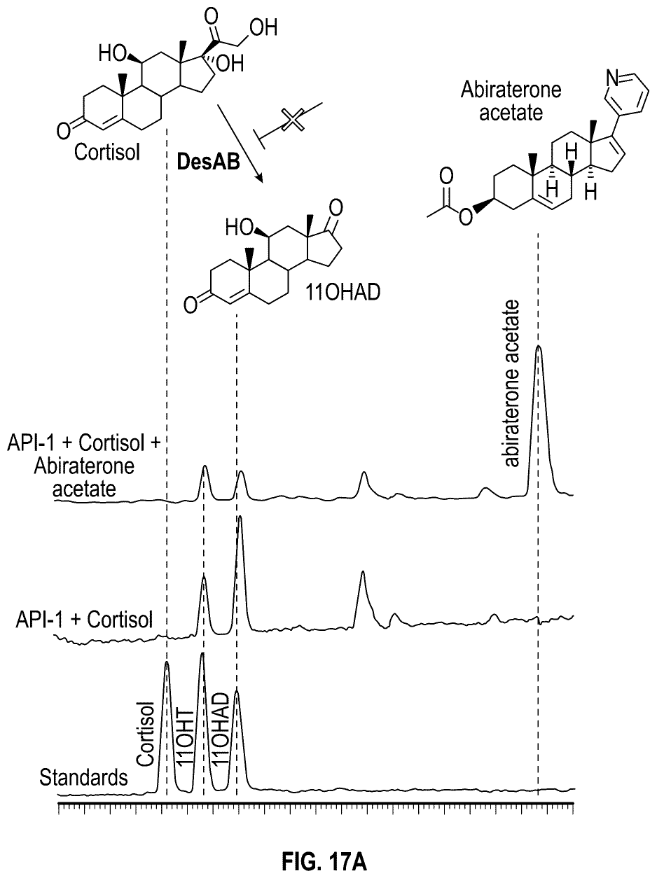

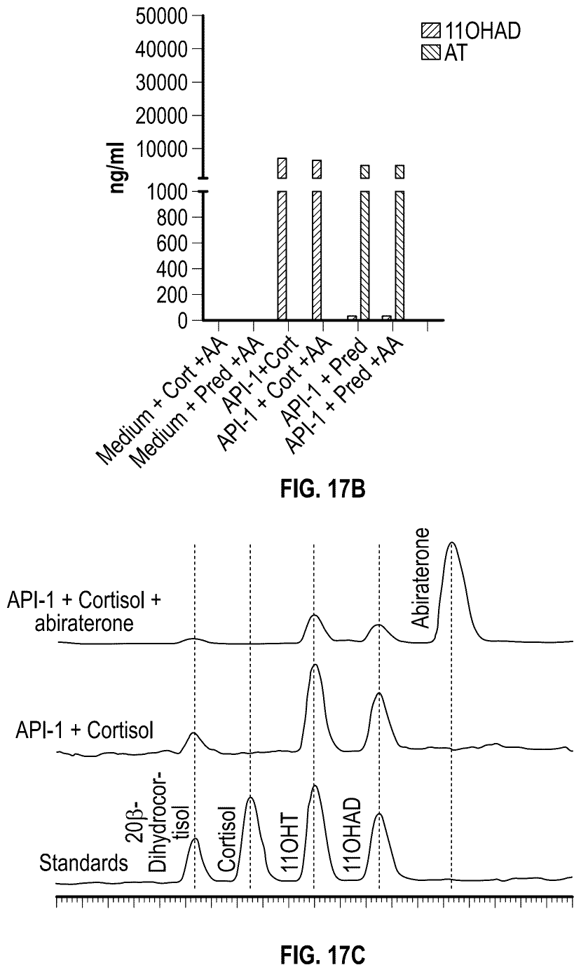

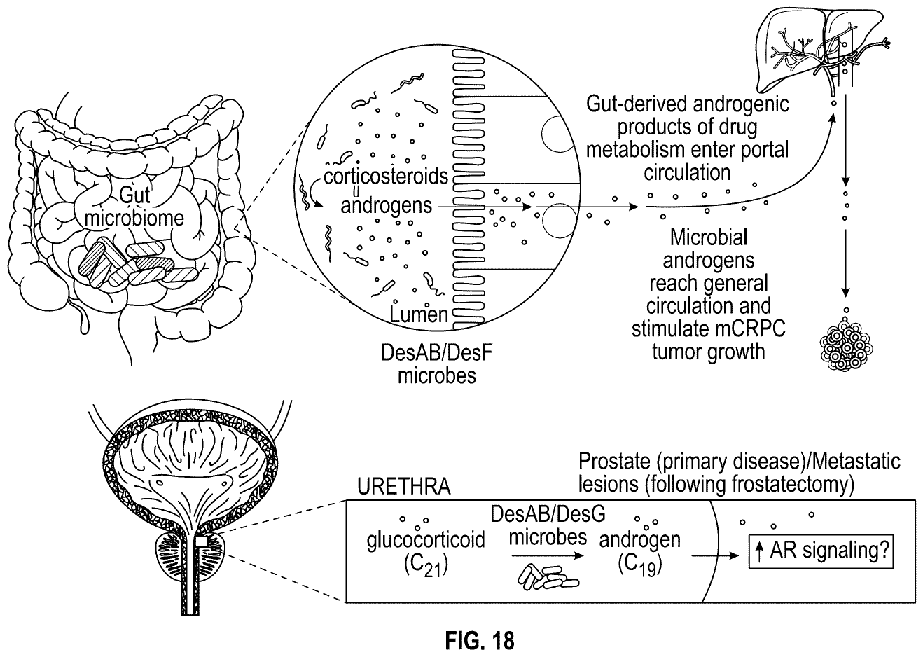

C. scindens ATCC 35704 (Csci35704) expresses steroid-17,20-desmolase encoded by the desAB genes and C. scindens VPI 12708 (Csci12708) can convert androstenedione (AD) to epiT. We examined if co-culture of both strains in the presence of 11-deoxycortisol (11DC) would yield epiT with a stable AD intermediate. Co-culture of Csci12708 and Csci35704 yielded the conversion of 11DC (RT 3.81 min; 347.2 m/z) to both AD (RT 4.47 min; 287.2 m/z) and epiT (RT 4.60; 289.2 m/z) after 24 h (FIG. 1a, b). 11DC (0 h=45.54±0.59 μM) was depleted in 24 h, yielding AD (24 h=6.20±0.22 μM) and epiT (24 h=27.42±0.64 μM). We confirmed the formation of epiT from AD in pure cultures of Csci12708 by a combination of high-resolution LC/MS/MS, and proton and carbon NMR (FIG. 6). Endocrine pathways in steroidogenic tissues (e.g., adrenal gland and gonads) generate AD and epiT through pathways distinct from Csci strains. Indeed, DHEA is converted to AD via HSD3B2 or the lyase activity of CYP17A1 coupled with P450 oxidoreductase (POR) and cytochrome b5 (CYB5) generates AD from 17α-hydroxyprogesterone¹⁹. While the metabolic pathway for epiT formation by human steroidogenic pathways is unknown, it is speculated to derive from 5-androstene-3β,17α-diol rather than AD²⁰.

### Example 2 the Gut Microbial desF Gene Encodes a Novel 17α-HSDH

We next sought to identify the gene(s) encoding 17α-HSDH in Csi12708 responsible for catalyzing the conversion of AD to epiT. We performed comparative genomics between Csci35704 and Csci12708 to identify reductases unique to Csci12708 (FIG. 1c; FIG. 7). The strains share 35% of their genes (1916 ORFs) with 33% of genes (1,800 ORFs) unique to Csci12708 (FIG. 1c). We narrowed this list down to three protein families known to include HSDH enzymes: 25 belonging to the short chain dehydrogenase/reductase (SDR) family; 23 to the medium chain dehydrogenase/reductase (MDR) family; and 2 to the aldo-keto reductase (AKR) family²⁰. Of these, 18 SDR, 18 MDR, and 2 AKR proteins are unique to Csci12708 (FIG. 7).

Given this relatively large number of candidates, we opted to utilize genome-wide transcriptomics to identify candidates after the growth of Csci12708 in the presence of 50 μM 11β-hydroxyandrostenedione (11OHAD) (n=4) vs. uninduced controls (n=4) (FIG. 1d). The addition of 11OHAD significantly upregulated (3.07 log₂ FC; FDR 0.012) the expression of a single gene (GGADHKLB_00774) (FIG. 1d). This observation was reproducible since repeat transcriptome analysis with additional biological replicates (n=3) yielded the same overall result (FIG. 8). GGADHKLB_00774 is predicted to encode a 27-kDa short chain dehydrogenase/reductase (SDR) superfamily protein (FIG. 1e). Importantly, GGADHKLB_00774 is in the list of reductases found in Csci12708 but not in Csci35704 (FIG. 7). Since many HSDHs are represented in the SDR superfamily²¹, we cloned GGADHKLB_00774 in a pET51b(+) vector for overexpression of the recombinant streptavidin-tagged enzyme. With 10 nM purified rGGADHKLB_00774 (30.105 kDa determined by proteomics), AD was converted to epiT in the presence of NADPH (but not NADH) (FIG. 1f), establishing that GGADHKLB_00774 from Csci12708 encodes a novel NADPH-dependent 17α-HSDH in the SDR superfamily. We propose the name desF for this gene, which constitutes the first host-associated microbial gene encoding a steroid 17α-HSDH.

To investigate the predicted molecular mechanism of ligand binding in the enzyme DesF we utilized AlphaFold 2²² through its QwikFold interface in VMD²³. Using VMD, the structure predicted by AlphaFold were aligned with similar structures available in the Protein Data Bank (PDB). Following a previously established protocol⁸, we used homologous structures from the PDB to fit an NADP⁺ molecule into the binding pockets of DesF monomer. We obtained homologous structures (PDB IDs: 4ILK, 4EJ6, 4A2C, 3QE3, 3GFB, 2DQ4, 2DFV, 2D8A, 1 PL7, and 1E3J) using protein BLAST. The alignment and placement of the NADP⁺ molecule on the binding site was performed using VMD. Utilizing advanced options in QwikMD²⁴, the ligand structure was minimized in the pocket along with nearby protein residues, while most of the protein structure remained static.

The structure of epiT in DesF was also fitted to the most probable binding site using VMD. Docking was performed using a combined manual and computational approach, where VMD was used to position reference atoms, and NAMD²⁵ through its QwikMD interface was used to minimize the structure of the complex, using a previously established protocol⁸. The complex was then solvated and subjected to a 100 ns equilibrium MD simulation. Al-based predictions and MD simulations showed that both epiT and NADP⁺ occupy a mostly open cleft (FIG. 1G, 1H).

MD simulations can be used to investigate the stability of molecular systems and predict the behavior and function of proteins or protein domains. Here, after 100 ns of MD simulation, we observed that epiT, and NADP⁺ remained stable in the predicted catalytic cleft. Analysis of the MD trajectory revealed strong interactions between the ligands and serine, tyrosine, and lysine residues in DesF. SER144 and TYR157 formed stable interactions with the epiT molecule (FIG. 1I). Additionally, LYS161 was identified by our network analysis as a key residue in stabilizing NADP⁺ in its position.

Phylogenetic analysis indicates that DesF appears to be unique to C. scindens strains, with these strains in a cluster well separated from the other sequences analyzed (FIG. 9). Accordingly, amino acid identity drops precipitously to at most ˜50% identity in SDR family proteins in other taxa based on the BLAST results in NCBI. DesF shares only 20.2% amino acid identity with Mus musculus 17α-HSDH, which is in the aldo-keto reductase family (AKR)²⁶. We thus sought to determine the proportion of C. scindens strains that harbor desABC and desF genes. We sequenced the genomes of 14 C. scindens strains and obtained an additional 20 genomes from sequenced C. scindens strains from NCBI²⁷⁻²⁹. Interestingly, two clades of C. scindens strains were apparent in whole genome phylogeny (FIG. 10) with the two most studied strains, Csci35704 (Clade 1) and Csci12708 (Clade 2), being distinct. In Clade 1, 12 strains harbored desABC genes, with desF being absent. In Clade 2, 17 of 19 strains encode desF, and two strains, SL. 1.22 and S076 had both desABC and desF genes.

We then constructed metagenome assembled genomes (MAGs) from publicly available human metagenomes resulting in 225 C. scindens MAGs (Table 1). In the C. scindens MAGs, 20 had desABC, 97 had desF, and 4 had both desABC and desF (Table 1). This indicates that strain variation in the gut microbiomes of humans may identify a subset of individuals with a higher potential to convert glucocorticoids into androgens, including derivatives of epiT.

TABLE 1

| MAGs (Clostridium scindens) | DesA | DesB | DesC | DesF |
|---|---|---|---|---|
| MGYG000165712 | 0 | 0 | 0 | 0 |
| BackhedF_2015_{——}SID87_12M_{——}bin.39 | 0 | 0 | 0 | 0 |
| GCA_902373645.1_MGYG-HGUT-01303_genomic | 1 | 1 | 1 | 0 |
| MGYG000118803 | 0 | 0 | 0 | 0 |
| MGYG000141644 | 0 | 0 | 0 | 1 |
| CokerMO_2019_SRR8692181_bin.17 | 0 | 0 | 0 | 1 |
| MGYG000081734 | 0 | 0 | 0 | 1 |
| MurphyR_2019_SRR7411324_bin.13 | 0 | 0 | 0 | 1 |
| MGYG000038545 | 0 | 0 | 0 | 1 |
| LoombaR_2017_{——}SID1048_bav_{——}bin.25 | 0 | 0 | 0 | 0 |
| MGYG000215940 | 0 | 0 | 0 | 1 |
| MGYG000259622 | 0 | 0 | 0 | 0 |
| MGYG000089331 | 0 | 0 | 0 | 1 |
| MGYG000027702 | 0 | 0 | 0 | 0 |
| MGYG000027307 | 0 | 0 | 0 | 0 |
| GCA_020892115.1_ASM2089211v1_genomic | 0 | 0 | 0 | 0 |
| FengQ_2015_{——}SID31883_{——}bin.30 | 0 | 0 | 0 | 1 |
| HeQ_2017_{——}SZAXPI029564-74_{——}bin.70 | 0 | 0 | 0 | 1 |
| MGYG000240459 | 0 | 0 | 0 | 1 |
| QinJ_2012_{——}T2D-016_{——}bin.41 | 0 | 0 | 0 | 1 |
| MGYG000179685 | 0 | 0 | 0 | 0 |
| CokerMO_2019_SRR8692210_bin.15 | 1 | 1 | 1 | 0 |
| MGYG000104828 | 0 | 0 | 0 | 0 |
| ljazUZ_2017_{——}S56_a_WGS_{——}bin.7 | 0 | 0 | 0 | 0 |
| FengQ_2015_{——}SID31537_{——}bin.55 | 0 | 0 | 0 | 0 |
| BackhedF_2015_ERR525992_bin.38 | 0 | 0 | 0 | 0 |
| GCA_020563525.1_ASM2056352v1_genomic | 0 | 0 | 0 | 1 |
| ljazUZ_2017_{——}S102_a_WGS_{——}bin.2 | 0 | 0 | 0 | 0 |
| MGYG000278088 | 0 | 0 | 0 | 1 |
| MGYG000082223 | 0 | 0 | 0 | 1 |
| MGYG000200365 | 0 | 0 | 0 | 0 |
| GeversD_2014_{——}SKBSTL016_{——}bin.45 | 0 | 0 | 0 | 0 |
| MGYG000001303 | 1 | 1 | 1 | 0 |
| RaymondF_2016_{——}P20E7_{——}bin.20 | 0 | 0 | 0 | 0 |
| MGYG000176351 | 0 | 0 | 0 | 1 |
| BackhedF_2015_{——}SID39_12M_{——}bin.41 | 0 | 0 | 0 | 0 |
| GCA_945908315.1_ERR1606358_bin.2_{—} | 1 | 1 | 1 | 0 |
| metaWRAP_v1.3_MAG_genomic | | | | |
| GCA_013304105.1_ASM1330410v1_genomic | 1 | 1 | 1 | 0 |
| MGYG000239495 | 0 | 0 | 0 | 0 |
| MGYG000209074 | 1 | 1 | 1 | 1 |
| MGYG000204864 | 0 | 0 | 0 | 1 |
| GCA_013304115.1_ASM1330411v1_genomic | 1 | 1 | 1 | 0 |
| MGYG000067279 | 0 | 0 | 0 | 0 |
| MGYG000078202 | 0 | 0 | 0 | 0 |
| MGYG000230541 | 0 | 0 | 0 | 0 |
| MGYG000146602 | 0 | 0 | 0 | 1 |
| NielsenHB_2014_{——}V1_UC11_0_{——}bin.24 | 0 | 0 | 0 | 0 |
| MGYG000212469 | 0 | 0 | 0 | 0 |
| MGYG000089002 | 0 | 0 | 0 | 0 |
| MGYG000033510 | 0 | 0 | 0 | 0 |
| HeQ_2017_{——}RSZAXPI003080-114_{——}bin.30 | 0 | 0 | 0 | 0 |
| MGYG000173046 | 0 | 0 | 0 | 1 |
| ljazUZ_2017_{——}S47_a_WGS_{——}bin.1 | 0 | 0 | 0 | 0 |
| MGYG000059009 | 0 | 0 | 0 | 1 |
| MGYG000246300 | 0 | 0 | 0 | 0 |
| BackhedF_2015_ERR525896_bin.18 | 0 | 0 | 0 | 0 |
| MGYG000233132 | 0 | 0 | 0 | 0 |
| MGYG000237452 | 0 | 0 | 0 | 0 |
| MGYG000288571 | 0 | 0 | 0 | 1 |
| MGYG000238973 | 0 | 0 | 0 | 0 |
| MGYG000176389 | 1 | 1 | 1 | 0 |
| MGYG000115572 | 0 | 0 | 0 | 0 |
| MGYG000108964 | 0 | 0 | 0 | 0 |
| MGYG000115658 | 0 | 0 | 0 | 1 |
| MGYG000009362 | 0 | 0 | 0 | 1 |
| MGYG000142659 | 0 | 0 | 0 | 1 |
| ljazUZ_2017_{——}S15_a_WGS_{——}bin.1 | 0 | 0 | 0 | 1 |
| MGYG000260488 | 1 | 1 | 1 | 0 |
| YuJ_2015_{——}SZAXPI015211-166_{——}bin.3 | 0 | 0 | 0 | 0 |
| MGYG000025974 | 0 | 0 | 0 | 1 |
| MGYG000183297 | 0 | 0 | 0 | 1 |
| MGYG000126456 | 0 | 0 | 0 | 1 |
| CokerMO_2019_SRR8692192_bin.27 | 0 | 0 | 0 | 0 |
| GCA_022137935.1_NA_genomic | 0 | 0 | 0 | 1 |
| CasaburiG_2019_SRR6277114_bin.5 | 0 | 0 | 0 | 1 |
| GCA_024463895.1_ASM2446389v1_genomic | 0 | 0 | 0 | 1 |
| MGYG000065110 | 0 | 0 | 0 | 0 |
| MGYG000125324 | 0 | 0 | 0 | 1 |
| MGYG000078756 | 0 | 0 | 0 | 1 |
| MGYG000239731 | 0 | 0 | 0 | 0 |
| XieH_2016_{——}YSZC12003_37190R1_{——}bin.30 | 0 | 0 | 0 | 1 |
| MGYG000151937 | 0 | 0 | 0 | 0 |
| GCA_905206435.1_ERR1600561-mag-bin.52_genomic | 0 | 0 | 0 | 0 |
| MGYG000158936 | 0 | 0 | 0 | 0 |
| GCA_020561885.1_ASM2056188v1_genomic | 0 | 0 | 0 | 1 |
| YuJ_2015_{——}SZAXPI015233-19_{——}bin.6 | 0 | 0 | 0 | 1 |
| MurphyR_2019_SRR7351869_bin.8 | 0 | 0 | 0 | 1 |
| FengQ_2015_{——}SID31137_{——}bin.48 | 0 | 0 | 0 | 0 |
| MGYG000256913 | 0 | 0 | 0 | 1 |
| ZellerG_2014_{——}CCIS24254057ST-4-0_{——}bin.15 | 0 | 0 | 0 | 1 |
| MGYG000232691 | 0 | 0 | 0 | 1 |
| MGYG000049970 | 0 | 0 | 0 | 0 |
| GeversD_2014_{——}SKBSTL008_{——}bin.83 | 0 | 0 | 0 | 1 |
| MGYG000054548 | 0 | 0 | 0 | 1 |
| GCA_019597925.1_ASM1959792v1_genomic | 0 | 0 | 0 | 1 |
| YuJ_2015_{——}SZAXPI003422-11_{——}bin.45 | 0 | 0 | 0 | 1 |
| MGYG000225303 | 0 | 0 | 0 | 1 |
| MGYG000237576 | 0 | 0 | 0 | 1 |
| GCA_945871535.1_SRR17382097_bin.48_{—} | 0 | 0 | 0 | 0 |
| metaWRAP_v1.3_MAG_genomic | | | | |
| FengQ_2015_{——}SID531333_{——}bin.47 | 0 | 0 | 0 | 0 |
| GCA_022777065.1_ASM2277706v1_genomic | 0 | 0 | 0 | 0 |
| NielsenHB_2014_{——}V1_UC16_0_{——}bin.1 | 0 | 0 | 0 | 1 |
| MGYG000180135 | 0 | 0 | 0 | 0 |
| MGYG000212934 | 0 | 0 | 0 | 1 |
| XieH_2016_{——}YSZC12003_37179_{——}bin.80 | 0 | 0 | 0 | 0 |
| MGYG000189753 | 0 | 0 | 0 | 0 |
| MGYG000137000 | 0 | 0 | 0 | 1 |
| GCA_022845835.1_ASM2284583v1_genomic | 1 | 1 | 1 | 0 |
| MGYG000260649 | 0 | 0 | 0 | 0 |
| MGYG000092190 | 0 | 0 | 0 | 1 |
| LiJ_2014_{——}V1.CD54-0_{——}bin.3 | 0 | 0 | 0 | 1 |
| HeQ_2017_{——}RSZAXPI003099-133_{——}bin.1 | 1 | 1 | 1 | 1 |
| GCA_017565985.1_ASM1756598v1_genomic | 0 | 0 | 0 | 0 |
| BackhedF_2015_{——}SID546_4M_{——}bin.12 | 0 | 0 | 0 | 0 |
| YuJ_2015_{——}SZAXPI017457-24_{——}bin.57 | 0 | 0 | 0 | 0 |
| MGYG000108396 | 0 | 0 | 0 | 1 |
| ljazUZ_2017_{——}S16_a_WGS_{——}bin.12 | 0 | 0 | 0 | 1 |
| MGYG000239782 | 0 | 0 | 0 | 1 |
| GCA_020563365.1_ASM2056336v1_genomic | 0 | 0 | 0 | 1 |
| ljazUZ_2017_{——}S48_a_WGS_{——}bin.2 | 0 | 0 | 0 | 0 |
| MGYG000032252 | 0 | 0 | 0 | 1 |
| MGYG000177750 | 0 | 0 | 0 | 0 |
| HeQ_2017_{——}SZAXPI029501-104_{——}bin.36 | 0 | 0 | 0 | 0 |
| MGYG000277528 | 0 | 0 | 0 | 0 |
| MGYG000093362 | 0 | 0 | 0 | 0 |
| MGYG000117810 | 0 | 0 | 0 | 0 |
| MGYG000288033 | 0 | 0 | 0 | 1 |
| MGYG000057334 | 0 | 0 | 0 | 1 |
| MGYG000228970 | 0 | 0 | 0 | 1 |
| MGYG000275853 | 0 | 0 | 0 | 1 |
| MurphyR_2019_SRR7351790_bin.1 | 0 | 0 | 0 | 1 |
| MGYG000049012 | 0 | 0 | 0 | 1 |
| MurphyR_2019_SRR7352056_bin.5 | 0 | 0 | 0 | 1 |
| GCA_016889005.1_ASM1688900v1_genomic | 1 | 1 | 1 | 0 |
| FengQ_2015_{——}SID31367_{——}bin.39 | 0 | 0 | 0 | 1 |
| MGYG000126162 | 0 | 0 | 0 | 1 |
| MGYG000230250 | 0 | 0 | 0 | 0 |
| ljazUZ_2017_{——}S14_a_WGS_{——}bin.19 | 0 | 0 | 0 | 1 |
| MGYG000245088 | 0 | 0 | 0 | 1 |
| ljazUZ_2017_{——}S46_a_WGS_{——}bin.2 | 0 | 0 | 0 | 0 |
| MGYG000260044 | 0 | 0 | 0 | 0 |
| MGYG000201716 | 0 | 0 | 0 | 0 |
| MGYG000004963 | 0 | 0 | 0 | 0 |
| FengQ_2015_{——}SID530450_{——}bin.66 | 0 | 0 | 0 | 1 |
| MGYG000100546 | 0 | 0 | 0 | 0 |
| MGYG000032531 | 0 | 0 | 0 | 0 |
| YuJ_2015_{——}SZAXPI017595-169_{——}bin.30 | 0 | 0 | 0 | 1 |
| MurphyR_2019_SRR7351692_bin.11 | 0 | 0 | 0 | 1 |
| MGYG000100111 | 0 | 0 | 0 | 0 |
| CokerMO_2019_SRR8692178_bin.1 | 1 | 1 | 1 | 1 |
| MGYG000286990 | 0 | 0 | 0 | 1 |
| MGYG000214544 | 0 | 0 | 0 | 0 |
| MGYG000196552 | 0 | 0 | 0 | 0 |
| MGYG000230595 | 0 | 0 | 0 | 0 |
| GCA_000471845.1_ASM47184v1_genomic | 0 | 0 | 0 | 0 |
| MGYG000234009 | 0 | 0 | 0 | 1 |
| GCA_020560435.1_ASM2056043v1_genomic | 0 | 0 | 0 | 1 |
| MGYG000217487 | 0 | 0 | 0 | 0 |
| ZellerG_2014_{——}CCIS88007743ST-4-0_{——}bin.21 | 0 | 0 | 0 | 1 |
| LomanNJ_2013_{——}OBK1196_{——}bin.13 | 1 | 1 | 1 | 0 |
| GCA_945830785.1_SRR5240736_bin.6_{—} | 1 | 1 | 1 | 0 |
| metaWRAP_v1.3_MAG_genomic | | | | |
| MGYG000234569 | 0 | 0 | 0 | 1 |
| VincentC_2016_{——}MM063_{——}bin.1 | 0 | 0 | 0 | 0 |
| NielsenHB_2014_{——}V1_CD7_4_{——}bin.41 | 0 | 0 | 0 | 0 |
| HeQ_2017_{——}SZAXPI029463-136_{——}bin.44 | 0 | 0 | 0 | 1 |
| MGYG000175564 | 0 | 0 | 0 | 0 |
| GCA_020555615.1_ASM2055561v1_genomic | 1 | 1 | 1 | 1 |
| GCA_013304085.1_ASM1330408v1_genomic | 0 | 0 | 0 | 1 |
| MGYG000067284 | 0 | 0 | 0 | 0 |
| MGYG000091745 | 0 | 0 | 0 | 0 |
| MGYG000027911 | 0 | 0 | 0 | 1 |
| MGYG000179895 | 0 | 0 | 0 | 0 |
| ljazUZ_2017_{——}S149_a_WGS_{——}bin.14 | 0 | 0 | 0 | 0 |
| NielsenHB_2014_{——}V1_UC11_5_{——}bin.53 | 0 | 0 | 0 | 0 |
| BackhedF_2015_ERR525961_bin.14 | 0 | 0 | 0 | 0 |
| LoombaR_2017_{——}SID5639_uuc_{——}bin.9 | 0 | 0 | 0 | 1 |
| MGYG000276115 | 0 | 0 | 0 | 0 |
| GCA_022845815.1_ASM2284581v1_genomic | 1 | 1 | 1 | 0 |
| MGYG000253462 | 0 | 0 | 0 | 0 |
| MGYG000154256 | 0 | 0 | 0 | 0 |
| FengQ_2015_{——}SID531403_{——}bin.12 | 0 | 0 | 0 | 0 |
| MGYG000019508 | 0 | 0 | 0 | 0 |
| YuJ_2015_{——}SZAXPI003424-12_{——}bin.59 | 0 | 0 | 0 | 1 |
| MGYG000243613 | 0 | 0 | 0 | 1 |
| LiJ_2014_{——}V1.CD3-0-PN_{——}bin.36 | 0 | 0 | 0 | 0 |
| YuJ_2015_{——}SZAXPI003415-12_{——}bin.8 | 0 | 0 | 0 | 1 |
| MGYG000147437 | 0 | 0 | 0 | 1 |
| HeQ_2017_{——}SZAXPI029483-78_{——}bin.3 | 0 | 0 | 0 | 1 |
| BackhedF_2015_{——}SID577_12M_{——}bin.28 | 0 | 0 | 0 | 0 |
| MGYG000127397 | 0 | 0 | 0 | 1 |
| GCA_000154505.1_ASM15450v1_genomic | 1 | 1 | 1 | 0 |
| GCA_024125195.1_ASM2412519v1_genomic | 0 | 0 | 0 | 1 |
| LiuW_2016_{——}SRR3992985_{——}bin.73 | 0 | 0 | 0 | 0 |
| MGYG000156646 | 0 | 0 | 0 | 0 |
| MGYG000014135 | 0 | 0 | 0 | 1 |
| MGYG000250872 | 0 | 0 | 0 | 1 |
| MGYG000084316 | 0 | 0 | 0 | 0 |
| GeversD_2014_{——}SKBSTL041_{——}bin.56 | 0 | 0 | 0 | 0 |
| MGYG000060898 | 0 | 0 | 0 | 0 |
| XieH_2016_{——}YSZC12003_37400_{——}bin.34 | 0 | 0 | 0 | 0 |
| YuJ_2015_{——}SZAXPI003428-6_{——}bin.7 | 0 | 0 | 0 | 1 |
| GCA_020562885.1_ASM2056288v1_genomic | 0 | 0 | 0 | 1 |
| GCA_009696415.1_ASM969641v1_genomic | 0 | 0 | 0 | 0 |
| MGYG000229429 | 0 | 0 | 0 | 0 |
| GCA_004295125.1_ASM429512v1_genomic | 1 | 1 | 1 | 0 |
| Baumann-DudenhoefferAM_2018_SRR7217830_bin.1 | 0 | 0 | 0 | 0 |
| MGYG000257293 | 0 | 0 | 0 | 1 |
| GCA_945875235.1_ERR1855542_bin.22_{—} | 1 | 1 | 1 | 0 |
| metaWRAP_v1.3_MAG_genomic | | | | |
| GCA_009684695.1_ASM968469v1_genomic | 0 | 0 | 0 | 0 |
| MGYG000043161 | 0 | 0 | 0 | 1 |
| MGYG000191948 | 0 | 0 | 0 | 0 |
| MGYG000235414 | 0 | 0 | 0 | 0 |
| MGYG000107506 | 0 | 0 | 0 | 0 |
| MGYG000183293 | 0 | 0 | 0 | 0 |
| MGYG000274932 | 0 | 0 | 0 | 1 |
| MGYG000115790 | 0 | 0 | 0 | 0 |
| ParnanenK_2018_SRR5723857_bin.3 | 0 | 0 | 0 | 0 |
| MGYG000197807 | 0 | 0 | 0 | 1 |
| MGYG000190421 | 0 | 0 | 0 | 0 |
| QinJ_2012_{——}DLF012_{——}bin.15 | 0 | 0 | 0 | 0 |
| MGYG000161697 | 0 | 0 | 0 | 0 |
| GCA_004558675.1_ASM455867v1_genomic | 0 | 0 | 0 | 0 |
| BackhedF_2015_ERR526080_bin.17 | 0 | 0 | 0 | 0 |
| BackhedF_2015_{——}SID546_12M_{——}bin.24 | 0 | 0 | 0 | 0 |
| | 20 | 20 | 20 | 97 |

### Example 3 EpiT Serves as an AR Agonist that Promotes Prostate Cancer Cell Proliferation

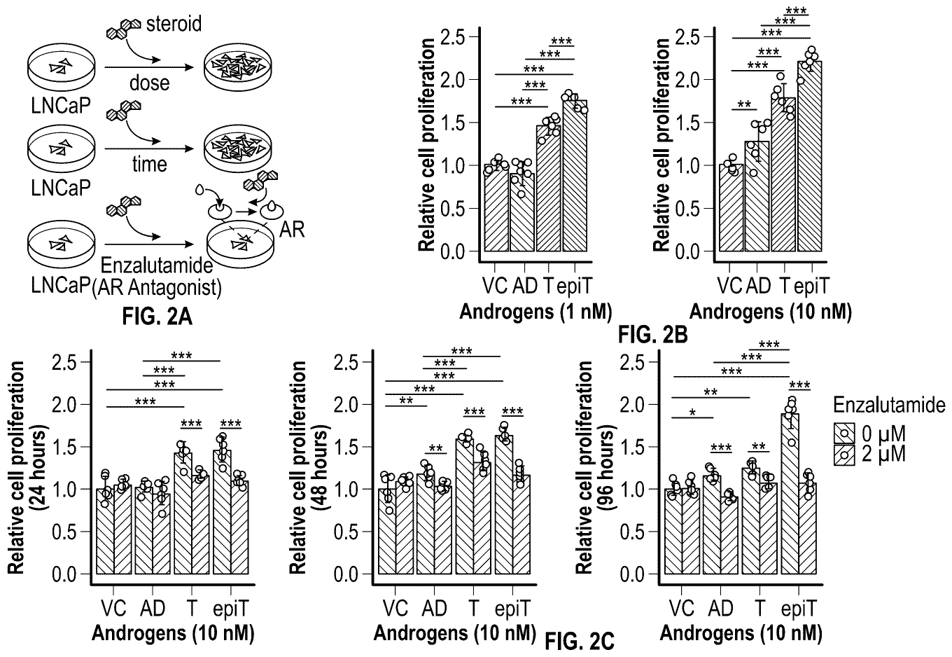

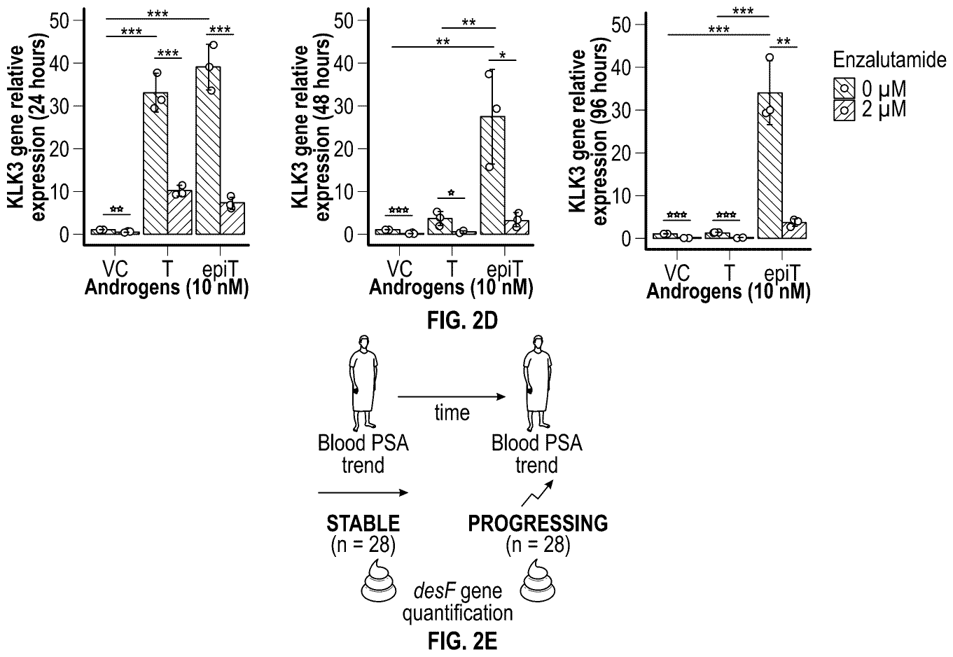

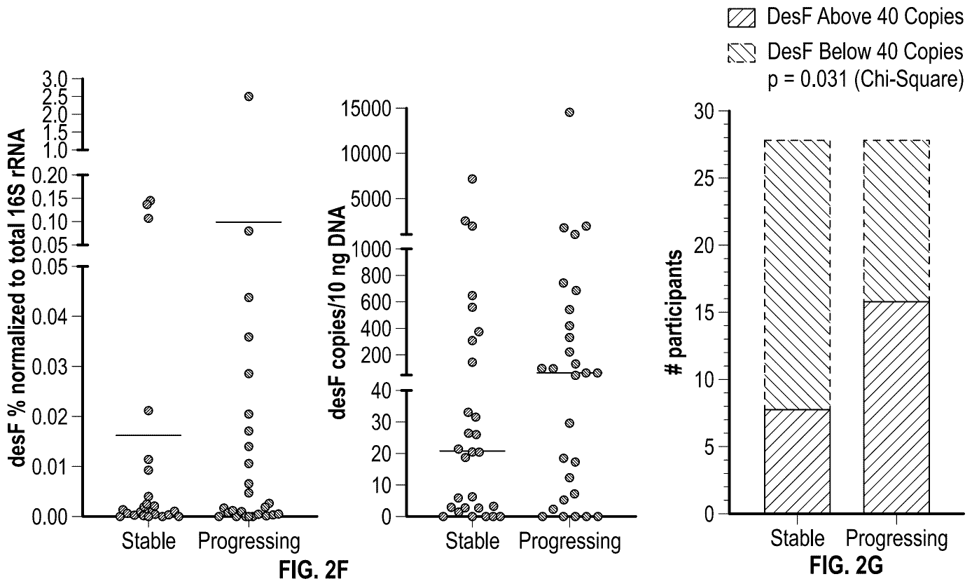

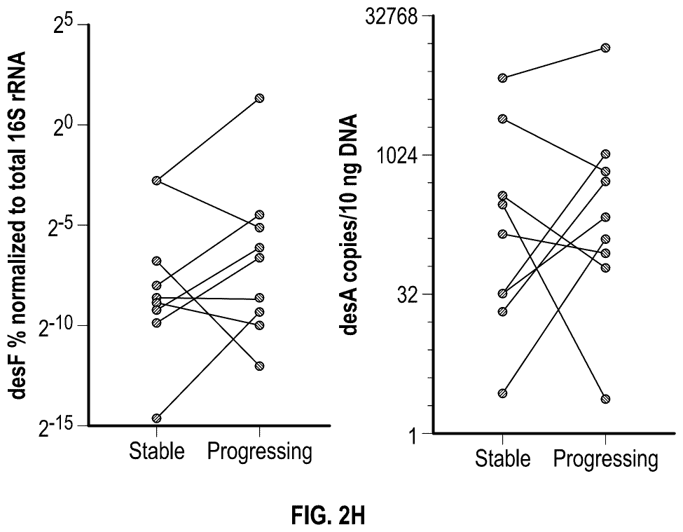

EpiT is regarded as an “antiandrogen” that is expected to bind to and antagonize AR and reduce prostate cancer cell growth³⁰. This dogma has been challenged with a recent study indicating that epiT instead serves as an AR agonist in a reporter cell line³¹. Circulating epiT is measured in the low nanomolar concentrations, with epiT/T ratios of 0.1 for women and 1 for men³². However, little evidence in the literature has examined epiT for its potential to alter cell physiology via nuclear AR³⁰. We thus compared the 96-h growth of androgen-sensitive prostate cancer cells (LNCaP) grown in charcoal-stripped medium in the presence of either 1 nM or 10 nM AD, T, and epiT to a vehicle control (VC; 0.5% v/v methanol) (FIG. 2A). As expected, at 1 nM, T caused significant proliferation (1.46±0.11 fold; P=2.0×10⁻⁰⁷) relative to VC (n=6); while the androgen-precursor, and non-AR ligand, AD, did not (0.90±0.14 fold; P=0.123) (FIG. 2B). Unexpectedly, epiT at 1 nM also caused significant proliferation relative to both VC (1.76±0.079 fold; P=1.1×10⁻¹⁰) and T (P=8.2×10⁻⁰⁵) (FIG. 2B). A dose-dependent effect was also observed such that proliferation caused by T at 10 nM increased significantly with respect to T at 1 nM (P=0.0024) (FIG. 2B). This pattern continued with epiT treatment at 10 nM in that proliferation was significant relative to 1 nM epiT (P=2.0×10⁻⁰⁵). EpiT treatment at 10 nM caused proliferation significantly above all other treatment groups (VC, P=1.4×10⁻¹⁰; AD, P=7.8×10⁻⁰⁹; T, P=0.00018) (FIG. 2B).

We next examined LNCaP proliferation over three time points. At 24 and 48 h, both T and epiT caused significant proliferation relative to AD (T, 24 h, P=1.9×10⁻⁰⁵; epiT, 24 h, P=1.3×10⁻⁰⁵; T, 48 h, P=1.6×10⁻⁰⁶; epiT, 48 h, P=4.2×10⁻⁰⁷) and VC (T, 24 h, P=1.3×10⁻⁰⁵; epiT, 24 h, P=1.3×10⁻⁰⁵; T, 48 h, P=1.0×10⁻⁰⁸; epiT, 48 h, P=5.4×10⁻⁰⁹) but were not significantly different in their effects relative to each other (FIG. 2C). At 96 h, T-stimulated proliferation decreased, although proliferation remains significant relative to VC (1.25 fold±0.078; P=0.0012); however, proliferation caused by epiT at 96 h increases significantly relative to 24 and 48 h, and proliferation in the presence of epiT is highly significant relative to all other treatments (VC, P=6.7×10⁻¹¹; AD, P=1.3×10⁻⁰⁹; T, P=8.0×10⁻⁰⁹) (FIG. 2C). Since LNCaP cells express a mutant androgen receptor with broadened steroid-binding capacity³³, we repeated these experiments with androgen-responsive VCaP cells that express wild type AR³³. Significant proliferation (AD, 2 d, P=0.00035; T, 2 d, P=8.5×10⁻⁰⁷; epiT, 2 d, P=1.9×10⁻⁸; AD, 4 d, P=1.4×10⁻¹⁰; T, 4 d, P=3.6×10⁻¹¹; epiT, 4 d, P=3.6×10⁻¹¹; AD, 8 d, P<2.0×10⁻¹⁶; T, 8 d, P<2.0×10¹⁶; epiT, 8 d, P=9.6×10⁻¹⁴) was observed with 10 nM AD, T, and epiT after 2, 4 and 8 days of exposure (FIG. 11). These results demonstrate that epiT functions as an AR agonist that drives the proliferation of prostate cancer cells.

To determine whether epiT-induced proliferation requires AR agonism, we treated LNCaP cells with 2.0 μM enzalutamide, an AR competitive inhibitor and prostate cancer drug (IC₅₀ 21.4 nM for LNCaP cells)³⁴. Treatment with enzalutamide caused consistent and significant growth inhibition of LNCaP cells in the presence of 10 nM AD, T, or epiT at all time points, indicating that proliferation in all cases was AR-dependent (24 h, T, P=0.00078, epiT P=0.00017; 48 h, AD, P=0.0053, T, P=0.00025, epiT P=1.7×10⁻⁰⁶; 96 h, AD, P=2.1×10⁻⁰⁵, T, P=0.0022, epiT P=2.9×10⁻⁰⁶) (FIG. 2C). We confirmed AR-dependent gene expression through measurement of the AR downstream gene target prostate specific antigen (PSA)/kallikrein-3 (KLK3)³⁵. PSA expression at 96 h was increased to 33.92±7.26-fold by epiT but reduced to 3.77±0.86-fold by enzalutamide (FIG. 2D). PSA gene expression is elevated to 32.97±4.33-fold in the presence of T at 24 h but dropped significantly to 3.74±1.70-fold at 48 h and to 1.35±0.12-fold by 96 h. In contrast to T, epiT caused prolonged PSA gene expression throughout the time course. In all cases, PSA gene expression is significantly reduced by enzalutamide treatment (FIG. 2D). Taken together, our results strongly indicate that epiT has a potent androgenic function not recognized previously.

### Example 4 Measurement of Human Fecal desF Gene Indicates the Potential Physiological Importance of Gut Bacterial 17α-HSDH

Prednisone (a replacement glucocorticoid commonly given in combination with AA) can be converted to 1,4-androstadiene-3,11,17-trione (AT) by DesAB¹⁷. We now questioned whether DesF could convert AT to epiAT, and whether treatment with this compound would lead to prostate cancer cell proliferation (FIG. 12). We cultivated Csci12708 in the presence or absence of 50 μM AT, diluted the 0.2 μm-filtered, 72-h spent culture medium in sterile charcoal stripped RPMI medium, and added it to LNCaP cell culture at final concentrations of 0.1, 1.0, or 10.0 nM (FIG. 12A). We verified quantitative depletion of AT after 48 h of cultivation with Csci12708 (FIG. 12B, 12C). Compared to spent medium (no steroid added) and spent medium with added AT (spiked in control), spent medium in which Csci12708 converted AT to epiAT resulted in dose-dependent proliferation of LNCaP cells, which was significant at 10 nM (CL, 1.66±0.14 fold, P=1.4×10⁻⁰⁶) (FIG. 12D). This proliferation was ablated by enzalutamide treatment indicating that epiAT triggered proliferation through AR-dependent signalling (FIG. 12E).

We next determined whether bacteria carrying the desF gene are present in the gut microbiota of individuals undergoing treatment for advanced prostate cancer with abiraterone acetate plus prednisone (AA/P), and whether quantitatively, fecal desF levels correlate with response to AA/P. Quantitative PCR (qPCR) primers were designed to target the desF gene, and we performed qPCR analysis on 56 fecal samples collected from 44 individuals with advanced prostate cancer undergoing treatment with AA/P (FIG. 2E. We found that 84.1% of the donors (37/44) had detectable fecal desF. Although not statistically significant, both the percentage of desF detected normalized to total 16S rRNA (as a surrogate for total bacterial load) as well as the absolute copy number of fecal desF were elevated in samples taken while individuals were progressing on AA/P versus samples taken during AA/P response (stable) (FIG. 2F). We observed that the majority of the samples in the AA/P stable group were quantitatively below 40 copies of desF. When the samples were categorized as below 40 copies versus above, the AA/P progressing group had significantly more samples with >40 copies of the desF gene detected (Chi-square, P=0.031, FIG. 2G). Twelve of the donors had samples taken both while they were stable on AA/P and when their disease was progressing. We found that a subset of these individuals had substantial increases in fecal desF levels during PSA progression on AA/P (FIG. 2H). These results suggest that desF-mediated androgen production by the gut microbiota (e.g., epiT) and/or metabolism of a replacement glucocorticoid (e.g., P) given in combination with AA could plausibly influence treatment response to AA/P, at least in a subset of individuals.

### Example 5 Formation of T Derivatives from Glucocorticoids by a Bacterium Isolated from Prostatectomy Tissue

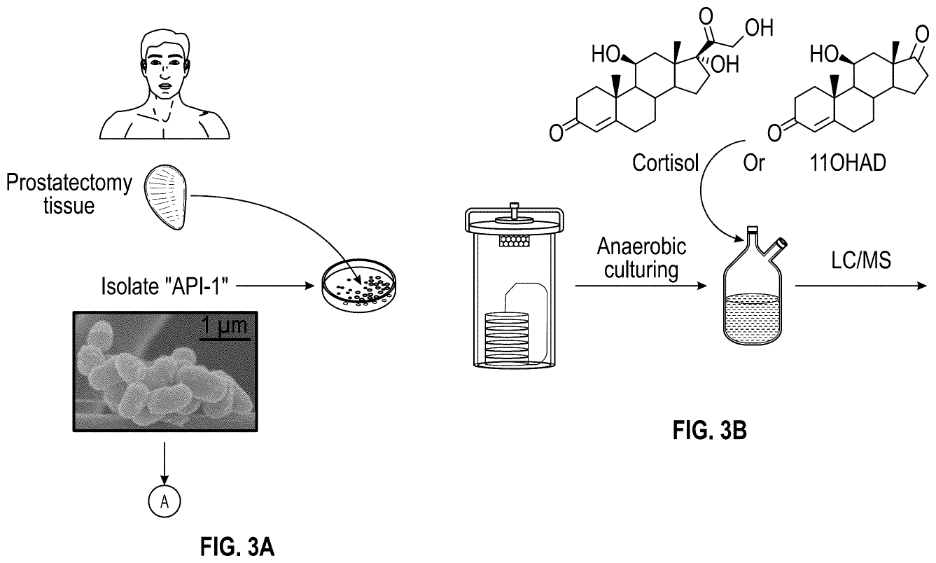

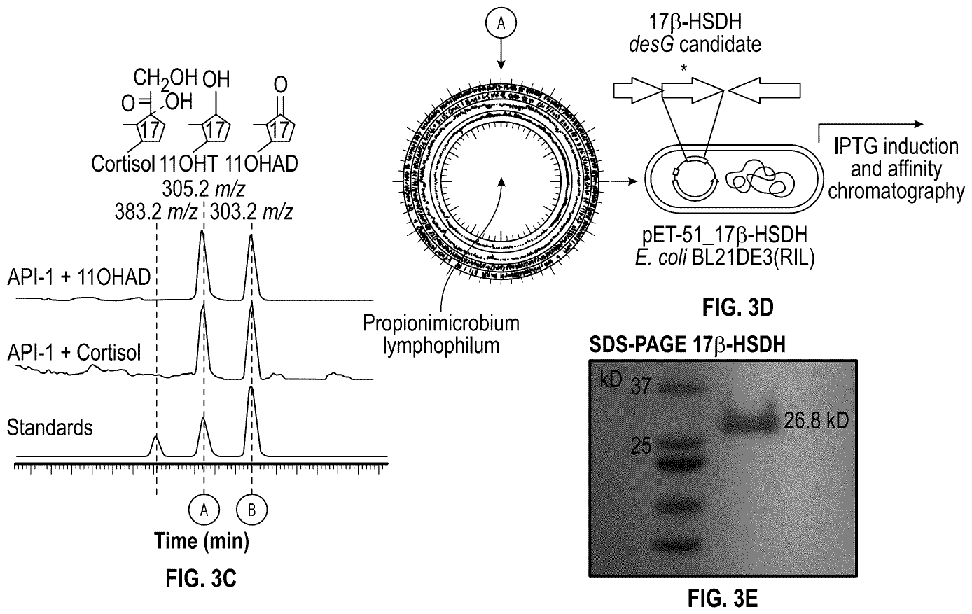

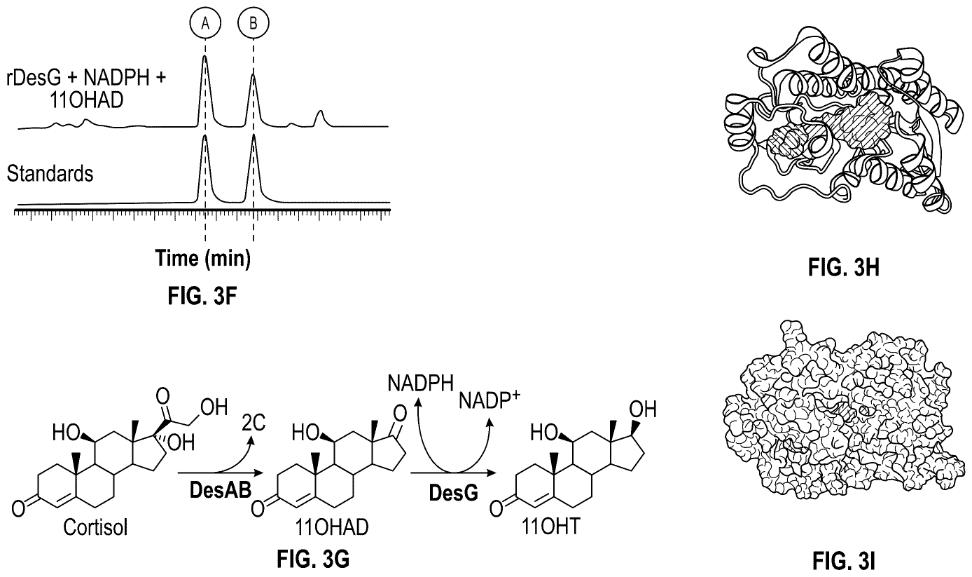

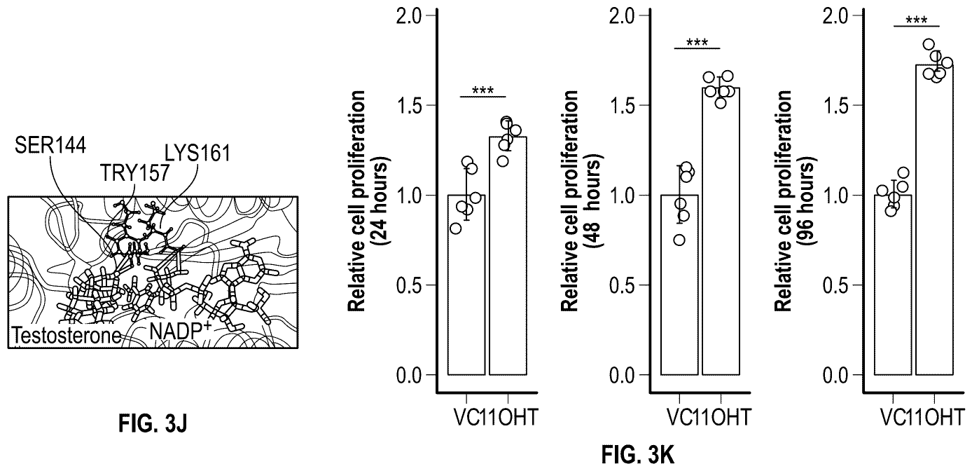

P. lymphophilum, a normal inhabitant of the urinary tract, and can harbor desAB genes¹⁷. We therefore sought to explore microbial steroid biotransformations by urinary tract isolates. After screening pure cultures established in a prior collection of bacterial isolates from prostatectomy tissue³⁶, we identified and cultured a strain of P. lymphophilum (strain API-1) capable of steroid metabolism (FIG. 3A, 3B; FIG. 13). We speculate that strain API-1 could have derived from prostatic fluid, or it could have been present in the prostatic urethra. Strain API-1 was found to generate products from cortisol (363.2 m/z; RT 3.20 min) consistent with 11OHAD (303.2 m/z; RT 3.63 min) and 11β-hydroxytestosterone (11OHT, 305.2; RT 3.42 min) that co-migrate with authentic standards (FIG. 3C). While side-chain cleavage of cortisol may be considered the “gateway reaction” in the formation of androgens from glucocorticoids by the urinary tract microbiota, the conversion of the 17keto (AD, 11KAD) to 17β-hydroxy (T, 11KT) is required to increase the “androgenicity” of these metabolites due to substrate-specificity by nuclear AR. To our knowledge, this is the first report of steroid 17β-HSDH activity by bacteria isolated from the urinary tract, let alone from prostate tissue. We sequenced and assembled a 2.3 Mb genome of P. lymphophilum API-1 (FIG. 3C). The desABE operon was located, providing an enzymatic basis for the conversion of cortisol to 11OHAD. We then searched for candidate 17β-HSDH genes in P. lymphophilum API-1, and a single candidate (ILDKDCJM_00761) in the SDR family appeared most probable based on genomic context (monocistron)³⁷. This gene is annotated as a “3β-hydroxycholanate dehydrogenase”, suggesting that the enzyme metabolizes steroids. This gene was cloned, overexpressed and affinity purified from E. coli BL21(DE3)RIPL (FIG. 3D). The Strep-Tactin affinity purified 26.8 kDa recombinant enzyme (10 nM) was incubated with NADPH and 11OHAD and 11OHT formation was confirmed by LC/MS (FIG. 3E, 3F). Thus, we have discovered the first microbial 17β-HSDH in a bacterium isolated from prostate tissue. This 17β-HSDH gene is part of the steroid-17,20-demolase pathway in P. lymphophilum API-1, so we propose to name this gene desG (FIG. 3G).

Using VMD, the structure of DesG was predicted by AlphaFold 2 and aligned with similar SDR PDB structures, as we had done with DesF, allowing us to minimize the ligand structure of NADP⁺ into the predicted binding pocket. The structure of testosterone in DesG was also fitted to the most probable binding site using VMD. MD simulation determined that in contrast to the open pocket observed with DesF, the results indicated that testosterone and NADP⁺ occupy a pocket that closes over the catalytic region in DesG (FIG. 3H, 3I). In DesG, LYS161 is predicted to play a role in stabilizing NADP⁺, while SER144 and TYR157 formed stable interactions with the testosterone molecule (FIG. 3J). Simulations indicated that the predominantly hydrophobic clefts in both DesF and DesG are crucial for maintaining the stability of the complex, with steroid molecules fluctuating minimally in the cleft. Based on the MD simulations, we propose that the serine and tyrosine residues stabilize the steroid to initiate the enzymatic reactions, while the lysine residue helps hold NADP⁺ in place in both DesF and DesG.

We then performed RNA-Seq analysis in the presence of cortisol (n=5) or 11OHAD (n=5) vs. DMSO vehicle control (n=5) with P. lymphophilum strain API-1. We did not observe differential gene expression between vehicle control and cortisol treatment (50 μM) with respect to desA (ILDKDCJM_00614; 0.73 log₂ FC; 0.193 FDR), desB (ILDKDCJM_00613; 0.81 log₂ FC; 0.107 FDR), or desG (ILDKDCJM_00761; 0.28 log₂ FC; 0.61 FDR). These results indicate that expression of steroid-17,20-desmolase genes in P. lymphophilum are not regulated by steroids, as is the case in the GI tract⁶, but rather the genes are constitutively expressed. This form of regulation may be important in the urinary tract due to the nanomolar levels of urinary steroids¹⁸ whose quantities are likely insufficient for sensitive inducible systems to evolve.

Protein phylogeny of the amino acid sequence of DesG revealed other taxa isolated in the urinary tract, but whose steroid metabolism remains unknown (FIG. 14). Such sequences, which display >77% ID with the DesG from P. lymphophilum, include Arcanobacterium urinimassiliense (BQ7117_RS04815), Vaginimicrobium propionicum (CZ356_RSO1445), and Sutterella wadsworthensis (HMPREF1255_RS00895). Phylogeny and SSN analysis of DesAB from the gut bacterium Csci35704 led to identification of A. urinimassiliense as a desAB harboring urinary bacterium³⁰⁴². To functionally sample from nearby neighbors of DesG, we cloned the synthesized genes predicted to encode 17β-HSDH in these species into pET51b(+) and expressed these recombinant proteins in E. coli BL21(DE3) (FIG. 15A-15C). After protein purification (FIG. 15C) and incubation of 10 nM enzyme with 200 μM NADPH and 50 μM AD. rBQ7117_RS04815 from A. urinimassiliense (GenBank Accession No. WP_073996445.1) (FIG. 15D) yielded T. Together with our current phylogenetic results, this finding indicates that strains of A. urinimassiliense are also capable of generating 11OHAD and 11OHT from cortisol, along with strains of P. lymphophilum reported in this study. These data confirm a two-step pathway for converting glucocorticoids into 11OHT (FIG. 3G). To determine androgenicity of 11OHT, we measured time-dependent proliferation of LNCaP cells. Compared to VC, 10 nM 11OHT caused significant (P <0.001) and prolonged (96 h) growth of LNCaP cells indicating that this microbial pathway in the urinary tract generates androgens (FIG. 3K). Similar proliferation of VCaP cells was observed in the presence of 10 nM 11OHT (2 d, P=1.9×10⁻⁰⁸; 4 d, P=1.3×10⁻⁰⁸; 8 d, P<2.0×10⁻¹⁶) relative to VC (FIG. 11).

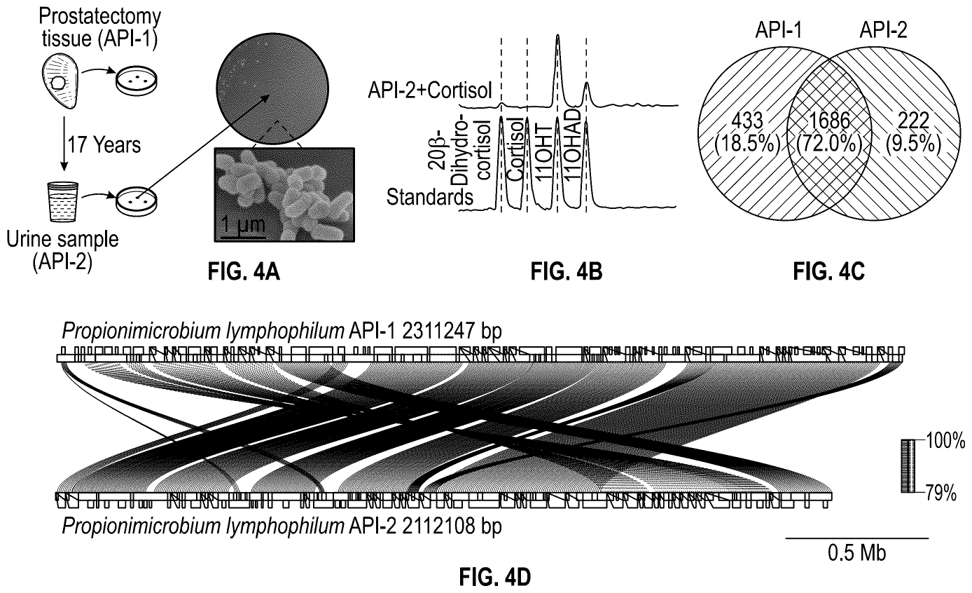

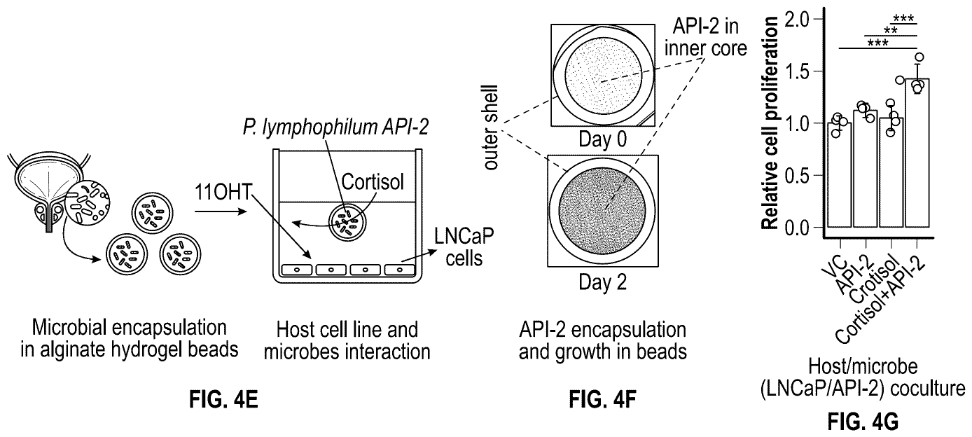

We then obtained a urine sample from the same male patient from whom we isolated P. lymphophilum API-1 from prostatectomy tissue to determine long-term colonization by strains similar to or evolved from P. lymphophilum API-1. This urine sample was collected approximately 17 years after prostatectomy (FIG. 4A). Upon isolating colonies from the urine sample, we screened for cortisol metabolism and obtained a bacterium capable of producing both 11OHAD and 11OHT (FIG. 4B). We sequenced the complete 2.1 Mb genome of this isolate and named it P. lymphophilum API-2. Comparative genome analysis indicated an average nucleotide identity (ANI) of 98.9% to API-1 with both strains sharing 72.0% of their predicted protein coding genes (FIG. 4C, 4D). These results indicate that some individuals experience long-term colonization of androgen-producing urinary tract bacteria.

### Example 6 Androgen Producing Urinary Microbes Promote Prostate Cancer Cell Proliferation

We recently developed and reported a microencapsulation technique using calcium alginate beads to co-culture bacteria and host cells allowing metabolic interaction without direct contact (FIG. 4E)^{38,39}. This bioengineered platform was utilized to determine the effect of cortisol metabolism on LNCaP cell proliferation. We first established the growth of P. lymphophilum API-2 in this platform in anaerobic bacterial growth medium (PYG broth) (FIG. 4f). LNCaP cell proliferation was determined in the co-culture platform in the presence of the bead encapsulated strain API-2+/−10 nM cortisol. Cells (50,000 cells/well) were seeded in a 24-well plate in RPMI 1640 media. Cell proliferation assay was carried out after 96 h. Significant proliferation was observed only in the presence of strain API-2 and cortisol (FIG. 4G). These data confirm that the metabolism of cortisol by prostatectomy tissue/urinary tract isolates promote prostate cancer cell growth by generating androgen metabolites capable of activating AR-signaling.

### Example 7 Culturomics of Androgen-Forming Taxa in Male Human Urine

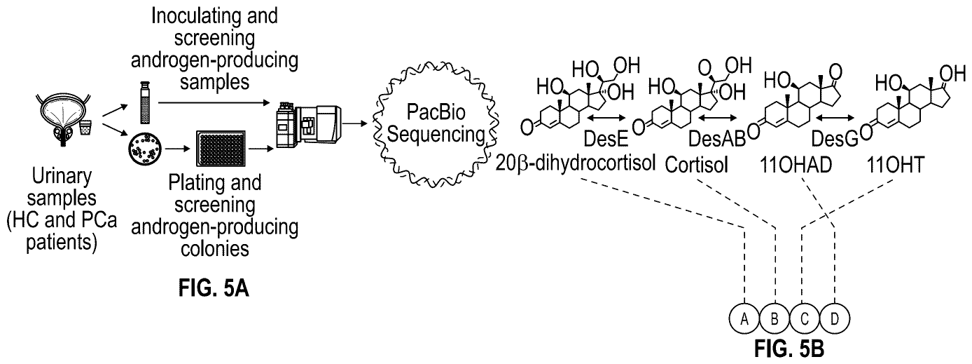

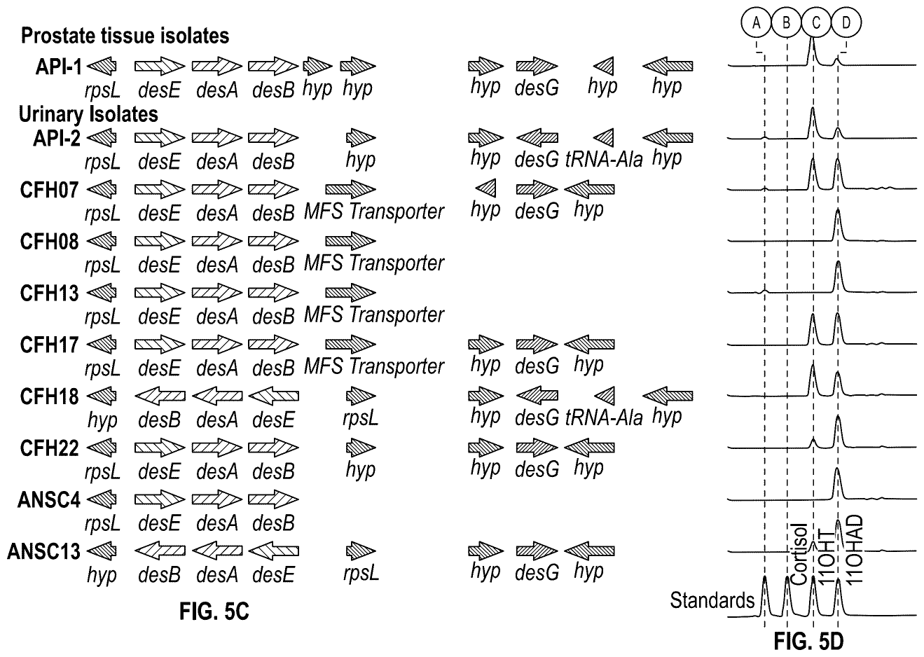

To identify additional androgen-forming urinary microbial taxa, we collected clean catch urine from 25 patients during a pre-biopsy visit to Carle Hospital Oncology, and clean catch urine from 14 age-matched healthy controls. We first screened urine samples for conversion of cortisol to 11OHAD (desAB activity) or 11OHT (desAB and desG activity) by diluting freshly collected urine in PYG broth. LC/MS analysis identified 11OHAD and/or 11OHT production in urine from 8 of 25 pre-biopsy samples, 4 of which were subsequently diagnosed with prostate cancer, and 2 of 14 healthy control samples (FIG. 5A; FIG. 13). Of the samples collected, 10 urine samples tested positive for 11OHAD and/or 11OHT production were plated (100 μL) on anaerobic Blood agar, Columbia agar and Schaedler agar. Colonies were cultivated in PYG broth in 96-well plates in the presence of 11DC in order to screen for desAB function, or 11OHAD to screen for desG function (FIG. 5A).

We obtained 9 P. lymphophilum isolates from urinary samples and sequenced their genomes (FIGS. 13, 16). We identified the desABE genes encoding bacterial desmolase (desAB) and steroid 20β-HSDH (desE)^{37,40} in all strains of P. lymphophilum (FIG. 5c). Strikingly, 6 of 9 strains of P. lymphophilum also had 17β-HSDH activity involved in conversion of 11OHAD to 11HT (FIG. 5C). We confirmed that the desG gene was found in the genomes of P. lymphophilum strains with both desmolase and 17β-HSDH activities, but not in the genomes of desmolase positive strains without 17β-HSDH activity (FIG. 5c). The desABE and desG genes and their metabolic activities in strain API-2 are consistent with that in strain API-1 (FIG. 3G; FIG. 5C, 5D).

### Example 8 Abiraterone does not Inhibit Androgen Production by Bacterial Desmolase

Host steroid-17,20-desmolase is encoded by CYP17A1 (the drug target of AA) and functions as an NADPH and 02-dependent P450 monooxygenase that facilitates adrenal corticosteroid and androgen biosynthesis through the side-chain cleavage and 17α-hydroxylation of pregnenolone¹⁹. CYP17A1 is inhibited by AA to treat prostate cancer through the inhibition of adrenal androgen synthesis. By contrast, bacterial steroid-17,20-desmolase (DesAB) functions under anaerobic conditions, through a predicted vitamin B1-dependent manner⁷ and may continue to function in the presence of AA, potentially contributing androgens from cortisone or P derivatives that may reduce the efficacy of AA/P therapy. We therefore tested AA (ICs₅₀ lyase activity=15 nM) for inhibition of the bacterial desmolase (DesAB) by pre-treating early log-phase cultures of P. lymphophilum API-1 in PYG broth containing cortisol or prednisone. We determined, in two independent labs, that therapeutic and physiologically relevant concentrations of AA do not inhibit bacterial desmolase from generating androgens from glucocorticoids (FIG. 17A, 17B). Moreover, the active form of A was not able to inhibit DesAB activity (FIG. 17C).

### Example 9

This study significantly advances our understanding of the genetic potential of host-associated microbiota to produce androgens from glucocorticoids and androstenedione. We identified new genes (desF, desG) that expand the steroid-17,20-desmolase pathway that previously included only the side-chain cleavage enzyme (DesAB) and the side-chain oxidoreductases 20α-HSDH (DesC) and 20β-HSDH (DesE)^{6,7,37,40} Specifically, our results reveal for the first time that (1) a novel bacterial pathway for conversion of AD (or cortisol derivatives) to epiT is encoded in the gut microbiome; (2) the end product, epiT, activates AR-dependent growth of LNCaP cells on par with T, indicating that epiT is a currently unrecognized AR agonist; (3) AA inhibits host production of adrenal androgens (AD, DHEA, 11OHAD) but not bacterial desmolase (desAB genes); (4) the desF gene is enriched in the fecal microbiota of individuals with advanced prostate cancer with disease progression on AA/P; and (5) enzymes in the epiT pathway may represent therapeutic targets for treatment of prostate cancer in some individuals in the same manner as the host steroidogenic enzymes are for drug targets. The discovery of the desABC and desF genes will allow direct quantification of microbial steroidogenic pathway genes that will complement fecal 16S rRNA profiling that can at most identify the abundance of “C. scindens”, strains which vary considerably in their capacity to metabolize steroids.

Moreover, we demonstrate for the first time that urinary tract bacteria, including a prostate tissue isolate, encode both desAB and the newly discovered desG gene that convert glucocorticoids (including prednisone) to T derivatives that promote prostate cancer cell proliferation. Urine is the main route of glucocorticoid excretion in humans, and glucocorticoids are measured in urine on the order of hundreds of nanomolar¹⁹. Based on this, we predict that it is possible that bacterial androgen production occurs locally in the prostatic urethra. Intriguingly, studies have shown that P. lymphophilum abundance in urine is associated with prostate cancer^{41,42} Long-term colonization of the urinary tract by androgen-producing bacteria may be an underrecognized promoter of the development and/or progression of prostate cancer in some individuals (FIG. 18).

### Example 10 Materials and Methods

### Bacteria, Cell Cultures, and Chemicals

Bacteria. Clostridium scindens ATCC 35704 (Csci35704) and Clostridium scindensVPI 12708 (Csci12708) were derived from in house 30% glycerol stock cultures and cultivated in anaerobic Trypticase Soy Broth (TSB) at 37° C. Individual colonies were picked from anaerobic TSB plates, DNA extracted, and identity confirmed by 16S rRNA gene sequence and confirmatory PCR targeting baiJ (Csci12708) or desA (Csci35704).

Cell culture. LNCaP and VCaP cells were obtained from ATCC and cultured in RPMI 1640 medium (Corning 10-040) and DMEM (ATCC 30-2002), respectively, and supplemented with 10% Fetal Bovine Serum (FBS) from GIBCO. Additionally, RPMI was supplemented with 10 mM HEPES buffer (Corning), 1 mM sodium pyruvate (Corning) and 4.5 g/L D-glucose (Sigma). For the androgen treatment experiments, cells were starved of androgens by cultivation in charcoal stripped FBS (GIBCO) medium. Cell passage number was kept below 25 for all experiments. Both cell lines were tested for mycoplasma contamination and authenticated using STR analysis in TEP facility (Cancer center at Illinois).

Steroids. Steroid (commercial sources) included: cortisol (Sigma); 11β-hydroxy-androstenedione (11OHAD, Steraloids, Newport, RI, USA); 11β-hydroxy-testosterone (11OHT, Steraloids); Epi-testosterone (epiT, Steraloids); 11-deoxycortisol (11DC, Sigma); Androstenedione (AD; Sigma); Testosterone (T, Sigma); 1,4-androstadiene-3,11,17-trione (AT, Sigma); 11-deoxycortisol-D5 (2,2,4,6,6-D5) (11DC-D5, Sigma); Androstene-3,17-dione-2,3,4-13C3 solution (AD-13C3, Sigma); Testosterone-D3 (16, 16, 17-d3) solution (T-D3, Sigma); 17-epi-testosterone-D3 (epiT-D3, Santa Cruz); Enzalutamide (Selleck); Abiraterone (A, MedChemExpress); Abiraterone acetate (AA, Sigma). T and AD were purchased in solution form (Sigma), evaporated with nitrogen, and redissolved in DMSO at the required concentration. EpiT, cortisol, 11OHAD, 11OHT, prednisone and AT were dissolved in DMSO or methanol. Enzalutamide was dissolved in DMSO.

### Bacterial Media Preparation

Brain Heart Infusion (BHI, BBL) broth was purchased and prepared based on the instructions. The Trypticase Soy Broth (TSB, BBL) was prepared as instructed with the addition of 5 g yeast extract, 1 g L-cysteine, 1 mg resazurin and 40 mL salt solution (1 L; 0.25 g CaCl₂)·2H₂O, 0.5 g MgSO₄·7H₂O, 1 g K₂HPO₄, 1 g KH₂PO₄, 10 g NaHCO₃, 2 g NaCl). Peptone Yeast Glucose (PYG) broth (modified) was prepared according to the DSMZ protocol. Briefly, each liter contains 5 g trypticase peptone, 5 g peptone, 10 g yeast extract, 5 g beef extract, 5 g glucose, 2 K₂HPO₄, 1 mL tween 80, 40 mL salt solution (see above), 1 mg resazurin, 0.2 mL vitamin K1 solution (5 mg/ml), 10 mL hemin solution (50 mg/100 mL), 1 g L-cysteine. Blood agar base (Sigma) was purchased and prepared according to the instructions with 6% (v/v) defibrinated sheep blood (Thermo Scientific) added. Schaedler agar (Sigma) was purchased and prepared as instructed. Columbia broth (BBL) was purchased and prepared as instructed with 15 g/L agar added to solidify the medium. The broth was made anaerobic by storage in Hungate tubes with 100% N₂ in the headspace. Plates were made anaerobic by storage in an atmosphere of 85% N₂:10% CO₂:5% H₂.

### Whole-Cell Steroid Conversion Assay

Csci35704 and Csci12708 were precultured in TSB. Afterwards, each fresh culture of these two strains was inoculated (0.5 mL) into fresh TSB with 50 μM 11DC. For sampling, 1-mL samples were collected at 0 and 24 h. The collected samples were clarified by centrifugation (13,300×g, 10 min; Thermo Scientific) and the supernatant fluid was used for subsequent analysis.

P. lymphophilium strains isolated from prostatectomy tissue or urine samples were cultured in anaerobic PYG broth. Log-phase cultures were transferred to fresh PYG broth containing 50 μM cortisol and incubated for 48 h. These samples were extracted and analyzed by LC/MS for 11-oxyandrogen formation as discussed below.

### Steroid Extraction

Two parts ethyl acetate and 1 part bacterial culture supernatant were thoroughly mixed by vortexing for 1 min. Next, the ethyl acetate layer was carefully collected and transferred to new tubes. The extraction process was repeated, and the collected top layers were evaporated with nitrogen gas and dissolved in 200 μL LC-MS grade methanol. For samples where substrates and end products were to be quantified, an internal standard mixture of 11DC-D5, AD-13C3, and epiT-D3 were added before extraction. The concentrations of 11DC, AD and epiT were normalized based on the under-curve area of the internal standards accordingly.

### Liquid Chromatography-Mass Spectrometry (LC-MS)

Samples were sent to the Mass Spectrometry Lab (University of Illinois at Urbana-Champaign, Urbana, Illinois, USA) for metabolite analysis using liquid chromatography-mass spectrometry (LC-MS). LC-MS for all samples was done on a Waters Aquity UPLC coupled with a Waters Synapt G2-Si ESI MS (Waters Corp., Milford, MA, USA). Chromatography was performed using a Waters Acquity UPLC BEH C18 column (1.7 μm particle size, 2.1 mm×50 mm) at a column temperature of 40° C. and an injection volume of 0.5 μL. For gradient elution, 2 mobile phases were used: mobile phase A contained 95% water, 5% acetonitrile, and 0.1% formic acid; mobile phase B contained 95% acetonitrile, 5% water, and 0.1% formic acid. Initially, mobile phase A was 100% for 0.5 min. Over the next 5.5 min, mobile phase B linearly increased, reaching 70% at 6 min. Then, mobile phase B increased to 100% in 1 min and maintained for 1 min. Afterwards, a steep reversal to the initial conditions was done within 0.1 min, and the running condition was maintained until the end at 10 min. The flow rate was 0.5 mL/min. The LC eluents were introduced into the mass spectrometer equipped with electrospray ionization (ESI) with a positive ion mode for steroid analysis. The following optimized conditions were used: capillary voltage of 3 kV, desolvation temperature of 500° C., cone voltage of 25 V, collision energy of 4 eV, collision gas helium, source temperature of 120° C., cone gas flow of 10 L/h, and desolvation gas flow of 800 L/h. The mass range was 50-2000 Da. Mass Lynx v4.1 (Waters) was used for chromatographs and mass spectrometry data analysis.

### Nuclear Magnetic Resonance (NMR)

AD metabolism was performed in Csci12708 cultures grown at 37° C. in anaerobic BHI medium in the presence of 25 μM of AD. Following overnight incubation, growth was quenched by adding 2× volume ethyl acetate. After vortexing for 1 min, the organic layer was collected and evaporated to dryness under N2 gas. Dried extracts were resuspended in 500 μL methanol. One hundred microliters were injected and run on high-performance liquid chromatography (HPLC, Agilent) using a C18 reverse phase column (Agilent Eclipse XDB-C18), with a 50:50 methanol:water mobile phase at a flow rate of 1 mL/min. The absorbance of steroid metabolites was monitored at 240 nm by UV-Vis detector spectroscopy. The Csci12708 androstenedione metabolite was fractionally collected and sent for NMR analysis. ¹H and ¹³C-NMR spectroscopic data were obtained on a JNM-ECA800 (JEOL, Ltd., Tokyo, Japan) instrument operated at 800 and 200 MHz, respectively, with CDCl₃ as the NMR solvent. Chemical shifts were expressed in d (ppm), and coupling constants J_{H,H} are given in Hz. ¹H-¹H nuclear overhauser effect spectroscopy (NOESY), ¹H-¹H correlation spectroscopy (COSY), ¹H-¹³C heteronuclear single-quantum correlation spectroscopy (HSQC), and ¹H-13C heteronuclear multiple-bond correlation spectroscopy (HMBC) spectra were obtained using gradient-selected pulse sequences. The ¹³C distortionless enhancement by polarization transfer (135°, 90°, and 45°) spectra were measured between CH₃, CH₂, CH, and coherence based on their proton environments.

### Computational Search of Reductases Unique to Csci12708

A total of 261 Csci12708 protein sequences (CP113781.1) annotated as dehydrogenases, reductases and NADH dependent proteins, were subject to screening with the NCBI/CDSEARCH (ncbi.nlm.nih.gov/Structure/cdd/wrpsb.cgi), looking for short chain dehydrogenases/reductases, medium chain reductases, and aldo/keto reductases family candidates. NCBI/BLAST was used to filter unique protein sequences by removing any hit found above ˜95% identity. The remaining 38 candidate's domain architecture was visualized with EMLB-EBI/InterPro (ebi.ac.uk/interpro/). Theorical molecular weight was estimated with Expasy algorithms (expasy.org/resources/compute-pi-mw). Similarity within sequences was tested with EMLB-EBI MUSCLE/Clustal2.1 (ebi.ac.uk/jdispatcher/msa/muscle).

### RNA-Seq Analysis

Csci12708 was incubated in BHI broth in the presence/absence of 50 μM of 11OHAD at 37° C. for 24 h. Cultures (10 mL) were pelleted by centrifugation (4,000×g). RNA was extracted as previously described⁶. Samples were sent to Roy J. Carver Biotechnology Center, DNA Services Laboratory (University of Illinois at Urbana-Champaign, Urbana, Illinois, United States) for library construction and sequencing. Total RNAs were run on a Fragment Analyzer (Agilent) to evaluate RNA integrity. The total RNAs were converted into individually barcoded polyadenylated RNAseq libraries with the Kapa HyperPrep mRNA kit (Roche, CA), with prior removal of rRNAs with the FastSelect Bacteria kit (Qiagen, CA). Libraries were barcoded with Unique Dual Indexes (UDI's) which have been developed to prevent index switching. The adaptor-ligated double-stranded cDNAs were amplified by with the Kapa HiFi polymerase (Roche). The final libraries were quantitated with Qubit (ThermoFisher) and the average cDNA fragment sizes were determined on a Fragment Analyzer. The libraries were diluted to 10 nM and further quantitated by qPCR on a CFX Connect Real-Time qPCR system (Biorad) for accurate pooling of barcoded libraries and maximization of number of clusters in the flowcell. The barcoded RNAseq libraries were loaded on one SP lane on a NovaSeq 6000 for cluster formation and sequencing. The libraries were sequenced as single-reads 100 nt in length. The fastq read files were generated and demultiplexed with the bc12fastq v2.20 Conversion Software (Illumina, San Diego, CA).

Read quality was evaluated using FastQC v0.11.8⁴⁵. SeqKit v2.0.0⁴⁶ was used to calculate the read number, sum of the read length, minimum read length, average read length and maximum read length for each sequencing file. Trimmomatic v0.39 was used to remove the adaptors and low-quality reads⁴⁷. SortmeRNA v4.3.6 was used to filter out ribosomal RNAs⁴⁸. Salmon v0.14.1 was used to do gene quantification⁴⁹ using C. scindens VPI 12708 genome as a reference²⁸. Gene abundance was filtered and normalized using the edgeR package⁵⁰. Differential gene expression analysis was performed using the limma package⁵¹

### Genomic DNA Isolation

Genomic DNA of C. scindens ATCC 35704, C. scindens VPI 12708 and P. lymphophilum API-1 were extracted using the QlAamp PowerFecal Pro DNA kit (Qiagen) according to the manufacturer's instructions. The extracted DNA was used to amplify the target genes for the heterologous expression of the potential candidates.

### Heterologous Expression and Purification of Potential 17α-HSDH and 17β-HSDH Proteins

The target inserts were amplified using the primers (ATATATGGATCCGATGAAGAATTTATTTGATC (SEQ ID NO:5); BamHI-HF; ATATATAAGCTTCTAAACAAGCGTCCAGCC (SEQ ID NO:6) HindIII-HF) synthesized by Integrated DNA Technologies (IDT, Coralville, IA, USA) and Phusion High Fidelity Polymerase (Stratagene, La Jolla, CA, USA). Inserts and the pET-51b(+) plasmid (Novagen, San Diego, CA, USA) were double digested using the appropriate restriction enzymes (NEB, Ipswich, MA, USA). The digested inserts and plasmids were ligated using the T4 DNA ligase. Recombinant plasmids were transformed into E. coli DH5a cells via heat shock at 42° C. for 30 seconds. E. coli DH5a were plated on lysogeny broth (LB) agar plates supplemented with 100 μg/mL ampicillin. Single colonies were picked up and transformed into 10 mL LB broth with 100 μg/mL ampicillin and grown for 10 h prior to plasmid extraction from cells. The plasmids were extracted using the QiAprep Spin Miniprep kit (Qiagen, Valencia, CA, USA). The extracted plasmids were transformed into E. coli BL-21 CodonPlus (DE3) RIPL chemically competent cells by heat shock method as mentioned above and cultured overnight at 37° C. on LB agar plates supplemented with 100 μg/mL ampicillin. Selected colonies were precultured in LB broth with 100 μg/mL ampicillin for 6 h at 37° C., and subsequently added to fresh LB medium (1 L), supplemented with 100 μg/mL ampicillin. When the OD₆₀₀ₙₘ reached 0.4, the incubation temperature was decreased to 25° C. and IPTG was added to each culture at a final concentration of 0.1 mM to induce the protein production during the 16-h incubation.

Subsequently, cells were pelleted and resuspended in 30 mL of binding buffer (10 mM Tris, 400 mM NaCl, 10 mM 2-mercaptoethanol, pH 8.0). To lyze cells, 750 μl of lysozyme (1 mg/mL) and 10 μl of benzonase nuclease (Sigma) were added and incubated on ice for 40 min. Afterwards, cells were physically lysed by passing through a French pressure cell press twice. Cell lysate was separated by centrifugation (13,300×g) at 4° C. for 30 min. The recombinant proteins in the soluble fraction were purified using Strep-Tactin resins (IBA Lifesciences) according to the manufacturer's instructions. The purified proteins were assessed by sodium dodecyl sulfate-polyacrylamide gel electrophoresis (SDS-PAGE). Protein concentrations were measured by Nanodrop 2000c spectrophotometer based on their extinction coefficients and molecular weights. Purified proteins were sent to Roy J. Carver Biotechnology Center, Proteomics Core (University of Illinois at Urbana-Champaign, Urbana, Illinois, United States) for protein confirmation and size determination.

### Enzyme Assays

Purified recombinant 17α-HSDH and 17β-HSDH activities were determined by mixing 10 nM enzyme, 50 μM substrate, and 200 μM cofactor (NADPH/NADP⁺) in phosphate-buffered saline. Samples were collected before and after the reactions. Steroids were extracted as mentioned above. Extracted samples were sent to the Mass Spectrometry Lab (University of Illinois at Urbana-Champaign, Urbana, Illinois, United States) for metabolite analysis using LC-MS.

### Proteomics

Samples of affinity purified DesF were digested with Trypsin (Thermo) using a CEM Discover Microwave Digestor (Matthews, NC) at 55° C. for 30 min. The digested peptides were extracted, lyophilized, and cleaned up using stage-tips⁵². LC/MS was performed using a Thermo Fusion Orbitrap mass spectrometer in conjunction with a Thermo RSLC 3000 nano-UPLC. The column used was a Thermo PepMap C-18 (0.75 mm×25 cm) operating at 300 nL/min and 40° C. The gradient was from 1% to 35% acetonitrile+0.1% formic acid over 45 min time interval. The mass spectrometer was operating in positive mode using data dependent acquisition method and collision induced dissociation for fragmentation at 35% energy. The raw data were analyzed using Mascot (Matrix Science, London, UK) and searches were made using a database consisting of all the desF and desG sequences. FDR (False Data Discovery Rate) using a decoy reversed sequence database was at 1%.

### Sample Collection and Prostate Cancer Study Cohort

All specimens were studied under a Johns Hopkins Medicine Institutional Review Board (IRB) approved protocol with written informed consent. Study participants (n=44) were instructed to collect a full stool sample followed by self-collection of rectal swabs (FLOQSwabs, Copan). The majority of the fecal samples used in this study were derived from rectal swabs. Seven of the fecal samples were swabs of stool. The samples were stored at −80° C. until time of DNA isolation.

All of the study participants had advanced prostate cancer and were undergoing treatment with AA/P. The majority of participants had castration resistant disease. Samples were categorized as “stable” (n=28) if the donor had circulating PSA levels at the time of sample collection that were decreasing or had not changed from the prior PSA measurement. Samples were categorized as “progressing” if the donor had circulating PSA levels that had increased at least 0.2 ng/mL from the nadir, and that continued to rise (n=27) or that was re-detectable after a prolonged period of being below the limit of detection (n=1). Twelve participants in the study had matched fecal samples collected while stable on AA/P and then while progressing on AA/P.

Fecal DNA Isolation and desF Quantitative PCR (qPCR)

DNA was isolated from fecal samples as previously described⁵³. Samples were diluted to 10 ng/μL in DNA-free water. For each 20 μL reaction, the following reagents were combined: 10 μL of iQ SYBR Green Mix (Cat No. 1708882, Bio Rad Laboratories), 2 μL of 10 μM Forward/Reverse Primer set, 6 μL DNA-free water, 1 μL of 2 ug/μL BSA (Cat No. B14, ThermoFisher Scientific), and 1 μL of 10 ng/μL DNA. Real-time PCR (qPCR) conditions and primers are outlined in Table 2A-C.

TABLE 2A

| | | |
|---|---|---|
| Temperature | Time | * 35 Cycles |

PCR Conditions for 17α-HSDH (desF):

| | | | |
|---|---|---|---|
| Forward Primer: | 95° C. | 3 min | * |
| 5′ GAGTACAAATGGCCCAAGGA 3′ | 95° C. | 30 sec | * |
| (SEQ ID NO: 7) | | | |
| Reverse Primer: | 65° C. | 30 sec | * |
| 5′ GCAGACACTCAGTACCGTTATC 3′ | 72° C. | 30 min | |
| (SEQ ID NO: 8) | | | |

Plate Read
Melt Curve 65-95° C.,
0.5° C./s

PCR Conditions for V6/16S:

| | | | |
|---|---|---|---|
| Forward Primer: | 95° C. | 3 min | |
| 5′ CAACGCGWRGAACCTTACC 3′ | 95° C. | 30 sec | |
| (SEQ ID NO: 9) | | | |
| Reverse Primer: | 53° C. | 30 sec | * |
| 5′ CRACACGAGCTGACGAC 3′ | Plate Read | | * |
| (SEQ ID NO: 10) | 72° C. | 30 sec | * |
| | Melt Curve 65-95° C., | | |
| | 0.5° C./s | | |

TABLE 2B

| | | | Amplicon |
|---|---|---|---|
| Gene¹ | | | (bp) |
| KLK3 | Forward | CGTGACGTGGATTGGTGC | 113 |
| | primer | (SEQ ID NO: 11) | |
| | Reverse | ACTGCCCTGCCACGAGAG | |
| | primer | (SEQ ID NO: 12) | |
| GAPDH | Forward | TCAAGGCTGAGAACGGGAAG | 117 |
| | primer | (SEQ ID NO: 13) | |
| | Reverse | TGGACTCCACGACGTACTCA | |
| | primer | (SEQ ID NO: 14) | |

¹Boerrigter et al. (2021). Molecular oncology, 15(9),
2453-2465.

TABLE 2C
PCR Conditions

| | Temperature | Time | cycles |
|---|---|---|---|
| activation | 50° C. | 2 min | 1 |
| polymerase dual lock activation | 95° C. | 2 min | 1 |
| and denaturation | | | |
| denaturation | 95° C. | 15 sec | 40 |
| annealing and extension | 60° C. | 1 min | |
| | Plate Read | | |
| | Melt Curve 60-95° C., 0.2° C./s | | |

Total copies of desF were estimated using standard curves with genomic DNA extracted from Csci12708. The qPCR efficiency of all qPCR assays was determined to be between 86-114%.

### Csci12708 Standard Curve Calculations

$$\frac{100 ng / μ L Csci 12708}{1 μ L} = \frac{1 genome}{4.302 \times {10}^{6} ng / genome} = 2.3 \times {10}^{7} genomes / μ L$$

$$V = \frac{1 \times {10}^{6} genomes / μ L \times 20 μ L}{2.3 \times {10}^{7} genomes / μ L} = 0.87 μ L Csci 12708 DNA + 19.3 μ L {H}_{2} O$$

A serial dilution was made from this stock solution as standard 1 down to standard 6 (1×10⁶ to 1×10²). All qPCR data were verified by running the PCR products on a 1.5% agarose DNA gel and confirmation of a band at the correct size.

Optimization of the desF Primer Set

To optimize the annealing temperature for the desF primer set, we ran a gradient annealing temperature plate with a positive control of genomic DNA from Csci12708, and negative controls of genomic DNA from Csci35704, the ATCC 20 strain mix (Cat No. MSA-1002, ATCC), human prostate DNA, and a no template control. Three gradient annealing temperature qPCR's using the above samples were run for a range of 52-54.9° C., 56.1-60° C. and 61-64° C. An annealing temperature of 65° C. was chosen based on the gradient PCRs and the positive and negative controls. The optimized cycling conditions were run with the positive control, negative controls, and a Csci12708 standard curve to confirm correct melt curve data and adequate qPCR efficiency. Lastly, the protocol was tested with 10 ng of Csci12708 combined with 10 ng of human prostate DNA, to ensure that human background DNA would not impact the specificity of the protocol towards bacterial genomic DNA in the clinical samples. A qPCR utilizing the Csci12708 standard curve both in the presence and absence of human prostate DNA was run. Both standard curves resulted in the same data, indicating that human background DNA would not impact the results.

Isolation of P. lymphophilum API-1 from Prostatectomy Tissue

The post-surgical prostatectomy specimen was placed in a sterile container following resection and transported to the grossing room. Here, a sterile field was assembled under a vertical laminar flow module (Envirco Corporation) for collection of tissue cores. A Biopty gun and sterile, single-use Biopty needles (18 gauge×16 cm, C.R. Bard) were used to obtain two cores from both the right and left lobes of the prostate. Biopsy needles were positioned from apex to base, sampling the posterior (peripheral) aspect of the prostate. The biopsy tissues were minced in sterile PBS, placed in a tube of thioglycolate broth, and cultured anaerobically at 37° C. Stock cultures were stored in 33% glycerol at −80° C.

### Patient Recruitment and Urine Sample Collection (CCIL Study)

To identify androgen-forming urinary microbial taxa, we consented and recruited 25 patients and 14 healthy individuals under the IRB #22383 (UIUC), Carle Hospital #18CCC1757. Inclusion criteria: No history of PCa, aged >50 years and <90. A BMI <35. Not having active diabetes. Exclusion criteria: Patients currently being treated for sexually transmitted infection, or urinary tract infection. Any patient taking antibiotics within the last month. Currently being treated for benign prostatic hyperplasia (BPH) i.e. with drugs such as alfuzosin (Uroxatral), doxazosin (Cardura), tamsulosin (Flomax), and terazosin (Hytrin) or abiraterone+prednisone or similar drugs. Unable or unwilling to give informed consent. Participants were given a urine collection kit and detailed instructions on how to properly collect a “clean/sterile catch”. The urine collection kit contained an alcohol swab to clean the urethra and tip of the penis. Participants were directed to catch mid-stream urine in sterile vials. At least 20 mL of urine was collected from participants. All urine samples were labelled with a unique, non-identifying code and were not derived from, or related to the participant's personal information. The urine samples were processed immediately for culturomics work.

### Urine Culturomics

To test urine samples for androgen-producing bacteria, 100-200 μL urine from each patient was screened in PYG broth containing 11DC and 11OHAD as substrates. 11DC was used to confirm side-chain cleavage activity (desAB) followed by 17β-HSDH activities in the urine yielding AD and T whereas 11OHAD conversion to 11OHT can identify 17β-HSDH activity when desAB-encoding microbes are lacking in the sample. LC/MS was performed to analyze metabolism of the substrates in these cultures.

Urine (100 μL) from each individual was plated (duplicate plates for each agar) to Blood agar, Columbia agar and Schaedler agar respectively. Each plate from each agar was incubated aerobically or anaerobically at 37° C. After 4-5 days, single colonies were picked using sterilized toothpicks to a 96-well plate containing PYG broth supplemented with 50 μM 11DC and 11OHAD. After incubation in an anaerobic chamber for 5 d, each column (50 μL/well) of 96 well plate was pooled to a single 1.5 mL centrifuge tube and extracted for the steroid. Columns positive for the steroid conversion were further processed to identify the individual positive well and the microbe carrying the conversion activity.

### Bacterial Colony Morphology and Scanning Electron Microscopy (SEM)

Androgen-producing bacterial isolates were cultured in PYG broth in an anaerobic chamber. During the log phase, these broth cultures were streaked on blood agar plates to determine colony morphology. For colony morphology, the blood agar plates were imaged under the Amscope microscope. Karnovsky's fixative, containing 2% glutaraldehyde and 2.5% paraformaldehyde was used to fix the bacterial isolates. Cultures (500 μL) in log phase were mixed with fixative (500 μL), vortexed, and then centrifuged 4,000×g for 5 min at 4° C. Supernatant was discarded, and the pellet was washed 3 times with fixative (500 μL) and then stored at 4° C. Samples were sent to Materials Research Laboratory Central Research Facilities (University of Illinois at Urbana-Champaign, Urbana, Illinois, United States) for SEM imaging.

Cortisol Metabolism by desAB Encoding Bacteria in the Presence of Abiraterone (A) and Abiraterone Acetate (AA)

Abiraterone binds irreversibly to CYP17A host enzyme and inhibit pregnenolone and progesterone conversion to AD and T. P. lymphophilum API-1 was cultured in an anaerobic chamber for 3 days. These cultures were then treated with A (50 μM) or VC (DMSO) to find the inhibitory effect of A on desAB enzyme activity. After 24 h, cortisol (50 μM) was added to cultures and incubated for 72 h. At the end, 200 μL cultures were extracted with ethyl acetate (as described above); extracts were dissolved in LC-MS grade methanol and subjected to LC/MS. In a separate study, API-1 was grown in RCB media under anaerobic conditions. 500 μL of API-1+50 μM of steroid (cortisol or prednisone)+1 uM of abiraterone acetate was added to a 7.5 mL RCB tube. This tube was grown anaerobically for 48 hours. The bacteria were then pelleted by centrifugation at 15,000×g for 3 min. The supernatant was collected and used for LC/MS/MS that was performed at the Analytical Pharmacology Shared Resource at Johns Hopkins University.

### Cell Proliferation Assay

Androgen-starved LNCaP and VCaP cells were trypsinized and seeded in 96-well plates at a density of 10,000 cells/well and then treated with enzalutamide (2 μM) or VC (DMSO; final concentration 0.2%). After 24 h, these cells were treated with VC (0.5% methanol) or 10 nM T, epiT, AD, and 11OHT. There were 6 replicates for each treatment. 3-(4,5-dimethylthiazol-2-yl)-5-(3-carboxymethoxyphenyl)-2-(4-sulfophenyl)-2H-tetrazolium (MTS) reagent (15 μL) was added to each well at the end of the experiment and incubated for 90 min. Absorbance was measured at 490 nm in a Biotek Synergy HT plate reader.

To determine if epiAT derived from AT metabolism by Csci12708 impacted proliferation of LNCaP cells, Csci12708 was cultured in anaerobic TSB (10 mL) containing 50 μM AT or VC (DMSO). For sampling, 1 mL cultures were collected at 0, 24, 48 and 72 h. Metabolites of AT to epiAT by Csci12708 were analysed by LC-MS. The spent cultures (72 h) were filtered through 0.2 μm-syringe filters prior to adding to LNCaP cell cultures. The filtered (sterilized) spent culture fluids were divided into the following treatment groups: 1) VC (TSB-DMSO spent culture); 2) AT spiked (TSB-DMSO spent culture with 50 μM AT added); and 3) epiAT (TSB-AT spent culture; Csci12708 encodes the desF gene and converts AT to epiAT during growth). LNCaP cells were seeded to 96-well plates as mentioned above and exposed to the equivalent of 0.1, 1 and 10 nM of AT or epiAT from the above treatment group 2 and 3, respectively. After 4 d of incubation, cell proliferation assay was conducted to compare cell proliferation between different treatments.

RNA Extraction and Gene Expression qPCR from Mammalian Cells

LNCaP cells incubated in cRPMI medium for 24 h were seeded in 12-well plates at a density of 100,000 cells/well. Cells were treated with VC (DMSO) or 2 μM enzalutamide. After 24 h, cells were treated with T, epiT or VC and incubated for an additional 24, 48, and 96 h. Cells were trypsinized and pelleted in 1.5-mL microcentrifuge tubes by centrifugation (500×g). Total RNA was extracted from cell pellets using GeneJET RNA Purification kit (ThermoScientific) where the lysis buffer was supplemented with 2% of 14.3 M β-mercaptoethanol. DNA contamination was removed from the extracted RNA using RapidOut DNA Removal Kit (ThermoScientific). Total RNA was measured using Nanodrop and 100 nM high-quality RNA was converted to cDNA with High-Capacity cDNA Reverse Transcription Kit (Applied Biosystems, ThermoFisher, Waltham, MA). Real-time PCR (StepOnePlus Real-Time PCR Systems; v 2.0 Applied Biosystems, Waltham, MA) was used to analyze differential gene expression. A total reaction volume of 20 μL in each well contained 0.5 μM forward and reverse primers (Table 2A-2C), 6 ng cDNA and PowerUp SYBR Green Master Mix (Applied Biosystems). Glyceraldehyde 3-phosphate dehydrogenase (GAPDH), a housekeeping gene was used as an exogenous reference to normalize transcription of PSA gene and the subsequent data was analyzed by the ΔΔCt method.

### Bacterial Encapsulation and Host/Microbe Coculturing

P. lymphophilium API-2 strain was cultured in PYG broth for 3 d in an anaerobic chamber. Encapsulation processes were followed by the reported method⁵⁴. Briefly, bacterial cultures in the exponential phase were collected and then thoroughly mixed with 3 wt % (w/v) of sodium alginate (Sigma) solution to a final concentration of 2.5% alginate. Alginate core beads were fabricated by dropping alginate/bacteria mixture into a 100 mM CaCl₂ solution while stirring for 10 min. The core beads encapsulating bacteria were transferred to a low concentration of alginate solution (less than 0.1 wt %) to fabricate a hydrogel-shell layer. The concentration of the alginate solution was increased very quickly by adding 3% of alginate solution to a final concentration of 0.5%. The reaction container was then vigorously shaken for 3 min to prevent core-bead aggregation. The process of shell fabrication was stopped by diluting the alginate solution when added an excessive amount of deionized (DI) water. The formed core-shell beads were transferred into a 0.01 M CaCl₂ solution under mild stirring for stabilization then washed with DI water. Hydrogel beads were immediately transferred to the anaerobic chamber and incubated in PYG for 4 d.

For the coculture study, P. lymphophilium API-2 beads and control (empty beads) were washed with DI sterilized water and conditioned in cRPMI medium for 6 h. The bacterial and control beads were then transferred to the 24-well plate. LNCaP cells maintained in cRPMI were sub-cultured to 24-well plates (50,000 cells/well). LNCaP/API-2 coculture platform was treated with cortisol (final concentration 10 nM) or VC (5% methanol) and incubated. After 96 h, beads were carefully removed from the cells using a 10-μl inoculation loop. Cell proliferation assay was conducted by adding 50 μl MTS reagent to each well, incubating for 90 min, and reading the absorbance (490 nm) using a Biotek Synergy HT Plate Reader.

### High-Molecular-Weight DNA Extraction

Urinary isolates were incubated at 37° C. in anaerobic PYG broth (10 mL). During log growth, cells in cultures (10 mL) were harvested by centrifugation (4,000 rpm) at 4° C. for 15 min). Cell pellets were washed 3 times with 1 mL TE buffer (10 mM tris, 1 mM EDTA, pH 7.6), and resuspended in TE buffer (450 μL) after the final washing. Bacterial lysis was carried out by the addition of lysozyme (2 mg/mL) with an incubation at 37° C. for 1 h. Next, proteinase K (1 mg/ml) was added, and the samples were incubated at 56° C. for 30 min. RNase A (4 μL, 100 mg/mL, Qiagen) was added to remove RNA and incubated at 25° C. for 2 min. After that, sodium dodecyl sulfate (SDS) was added (1%, Sigma) and the samples were incubated at 60° C. for 30 min. Equal volume phenol was added to denature proteins and then the samples were cleaned with 1 mL phenol/chloroform/isoamyl alcohol (25:24:1 vol/vol/vol) twice. Subsequently, 1/10 volume of 3 M sodium acetate and 2 volumes of ice-cold absolute ethanol were added with an overnight incubation at −20° C. to precipitate DNA. The precipitated DNA was purified by the addition of 1 mL 70% ethanol. After the evaporation of the ethanol, TE buffer (50 μL) was used to resuspend DNA. The size of the DNA was determined with 0.5% agarose gel. DNA concentrations were determined by the Nanodrop 2000c spectrophotometer.

### Whole Genome Sequencing and De Novo Assembly

Approximately 500 ng high-molecular-weight (HMW) DNA was sent to Roy J. Carver Biotechnology Center, DNA Services Laboratory (University of Illinois at Urbana-Champaign, Urbana, Illinois, United States) for whole genome sequencing. The HMW DNA was sheared with a Megaruptor 3 to an average fragment length of 13 kb. Sheared DNA was converted to a library with the SMRTBell Express Template Prep kit 3.0 from PacBio. The library was sequenced on a shared SMRTcell 8M on a PacBio Sequel lie using the CCS sequencing mode and a 30-hour movie time. CCS analysis was done in an instrument with SMRTLink V11.0 (PacBio) using the following parameters: ccs --min-passes 3 --min-rq 0.99.

Read quality was evaluated using FastQC v0.11.845. SeqKit v2.0.0⁴⁶ was used to calculate the statistics of the sequencing files for each microbial isolate. Flye v2.9⁵⁵ was used to assembly the reads (enough for 50 fold coverage) chosen using the parameters: --asm-coverage. Assembly quality and completeness were evaluated using QUAST v5.0.2⁵⁶ and BUSCO v5.5.0⁵⁷ respectively. Annotations were performed using Prokka v 1.14.6⁵⁸. CGView Server was used to make the circular genome maps⁵⁹. The 16S rRNA genes were extracted by SnapGene v6.2.1 and used to determine the species by doing the BLAST in NCBI.

### Comparative Genomics

Average nucleotide identity (ANI) was calculated using pyani v0.2.10⁶⁰ with the third-party tool MUMmer4⁶¹. Roary v3.13.0⁶² was used to generate a core gene alignment. The alignment was done via the parameters: -e-mafft. The best model for phylogenomics was calculated by using ModelTest-NG⁶³. RAxML-NG⁶⁴ was used to infer the bootstrapping tree with the parameter: --bs-trees autoMRE. An online tool (iTOL) was used for the tree display and annotation⁶⁵. The web application pyGenomeViz v0.4.0⁶⁶ was used to visualize the nucleotide sequence similarity with the GenBank format files as inputs based on MUMmer4. A Venn diagram was generated using ggvenn package⁶⁷.

### Metagenomics (MAGs)

Genomes from human gut microbiome datasets were downloaded from the following nine different sources: 32,277 genomes from Zeng et al. 2022⁶⁸, 1,200 genomes from Wilkinson et al. 2020⁶⁹, 120 genomes classified as C. scindens from Almeida et al. 2020⁷⁰, 1,381 genomes from Tamburini et al. 2021⁷¹, 154,723 genomes from Pasolli et al. 2019⁷², 4,997 genomes from Merrill et al. 2022⁷³, 2,914 genomes from Lemos et al. 2022⁷⁴, 4,497 genomes from Gounot et al. 2022⁷⁵, and 31 genomes from NCBI. The GTDB-Tk (version 2.1.1) classify workflow (classify_wf) was run on all 202,140 genomes, which resulted in the identification of 224 C. scindens genomes. Custom HMMs were generated for the proteins DesA, DesB, and DesF by using experimentally verified sequence. Briefly, in order to create the protein alignments needed to generate HMM profiles, muscle (5.1.linux64) was used with default parameters to align all amino acid sequences for each of the proteins followed by the hmmbuild function of the HMMER (version 3.3.2) package to generate the HMM profiles. Trusted HMM cutoffs were generated for each of the proteins based on the maximum F-scores based on searches with orthologous proteins. The 224 identified C. scindens genomes were translated into amino acid sequences using Prodigal (version 2.6.3). The generated HMM profiles were queried against the amino acid sequences for the 224 genomes using hmmsearch of the HMMER package with the flag --cut_tc.

### Phylogenetics (DesF and DesG)

The DesF protein sequence of C. scindens (accession number WP_220430766.1) and the DesG protein sequence of P. lymphophilum API-1 (this work) were used as queries for BLASTP searches against the NCBI's NR protein database. The protein sequences from the top one thousand (DesF) or five hundred (DesG) results were aligned using MUSCLE v. 5.1 with default parameters⁷⁶. Ambiguously aligned positions were removed with Gblocks v. 0.91 b⁷⁷, allowing up to 50% of the sequences in a column to contain gaps and using minimum length of a conserved block size of 5. Each phylogeny was inferred by IQ-TREE v. 2.2.2.6⁷⁸ using two independent runs, one thousand ultrafast bootstrap pseudoreplicates (optimized by NNI on the bootstrap alignment), one thousand SH-like approximate likelihood-ration test replicates, and extended substitution model selection (including Lie Markov models). Trees were edited in TreeGraph2⁷⁹, drawn in Dendroscope v. 3.8.10⁸⁰, and final cosmetic adjustments were performed in Inkscape (inkscape.org).

### Computational Structural Biology

Protein Modeling: The models for desF and desG protein structures were predicted using AlphaFold version 2.3.2²², employing all five available parameter sets to generate five models each, resulting in a total of 25 predictions per sequence. The QwikFold plugin in VMD²³ was used to set up the computational experiments and post-process the results, with calculations run using the Cybershuttle⁸¹. QwikFold was also utilized to align the models for visual inspection, addressing per-residue confidence as measured by pLDDT. The predicted aligned error (PAE) matrices were inspected to assess confidence in the predicted structures. Additionally, both structures were solved using a homology modeling strategy. The enzyme models were constructed using MODELLER⁸², which employs spatial restriction techniques based on 3D-template structures. The best model was selected by analyzing stereochemical quality using PROCHECK⁸³ and overall quality using the ERRAT server⁸⁴. VMD was then used to compare the structural models, and all predicted structures were used for the docking strategy.

Docking: Using BLAST⁸⁵, we obtained homologous structures (PDB IDs: 4ILK, 4EJ6, 4A2C, 3QE3, 3GFB, 2DQ4, 2DFV, 2D8A, 1 PL7, 1E3J) from the protein data bank (PDB). The design of the ligands (NADP+, testosterone, and epiT) was carried out using Molefacture, the small molecule design suite in VMD⁸⁶. The alignment and placement of both NADP+ and the steroid molecules (epiT in desF and testosterone in desG) in their binding sites were performed using VMD. Employing advanced run options in QwikMD²⁴ the structures of the ligands were minimized in the pockets along with nearby enzyme residues, while maintaining the structure of most of the enzyme as static. PyContact⁸⁷ was then used to analyze the contact interface.

Molecular Dynamics Simulations: MD simulations were performed using the GPU-accelerated NAMD²⁵ molecular dynamics package. The simulations assumed periodic boundary conditions in the NpT ensemble, with temperature maintained at 300 K using Langevin dynamics for temperature and pressure coupling, the latter kept at 1 bar. A distance cut-off of 12.0 Å was applied to short-range non-bonded interactions, while long-range electrostatic interactions were treated using the particle-mesh Ewald (PME) method⁸⁸. The equations of motion were integrated using the r-RESPA multiple time step scheme to update the van der Waals interactions every step and electrostatic interactions every two steps. The integration time step was set to 2 fs. Before MD simulations, the system underwent energy minimization and a short MD run protocol for 50,000 steps (1,000 steps of minimization followed by 1,000 steps of MD, repeated 25 times). An MD simulation with position restraints on the protein backbone atoms 7 Å or more away from the ligands was performed for 10 ns. To allow for total system relaxation and ensure ligand stability in the pockets, a 100 ns equilibrium simulation without external forces was performed.

MD Analysis: Analyses of MD trajectories were carried out using VMD and its plugins²³. Surface contact areas of interacting residues were calculated using PyContact⁸⁷. The generalized dynamical network analysis tool was employed to perform dynamical network analysis⁸⁹.

### Statistical Analysis

Statistical analyses were performed with R version 4.3.0⁹⁰. Data are shown as means±standard deviations (SD) when data are normalized. Data are shown in median and interquartile ranges when skewed. Categorical data are shown as counts and percentages. Differences between groups were analyzed by t-test when data are normal or Mann-Whitney U test when not normally distributed. Differences in categorical data were analyzed using chi-square test. The P values for multiple tests were corrected using Benjamini-Hochberg false-discovery rate (FDR). A P value s 0.05 was considered statistically significant.

DesF from Clostridium scindens
(SEQ ID NO: 1)
ATGAAGAATTTATTTGATCTGACAGGGAAGGTGGCGCTGATCACCGGCG
CTTCTTCTGGACTTGGAGTACAAATGGCCCAAGGACTCGCGGGGCAGGG
CGCCAAACTGGCGATTGTGGCCAGGAGGATGGATCGCCTGGAAAAGCTG
GCGAAAGAATTCGAGGATAACGGTACTGAGTGTCTGCCTGTGAAGTGTG
ATATTACGAAGGAAGAAGAGATCATTGAAATGGTAGATAAGGTGATATC
CCACTATGGGCAGATAGACATTCTGGTGAATAATGCAGGAATGGCATCT
GGAACTGCATCGGAGGATATGACGCTGGAGGAATGGGATAAGATTATAC
GGCTGAATCTGACCGGATCGTTTCTTGTATCCCGGGAAGTAGGGAAGCA
TATGATTGCCAGCCGATATGGAAAGATTATTAATACCTGCTCCATCCAG
GGAATCCGATGTACCATGGGGATGCCGGGAACTCCTTACAATTCGTCAA
AAGGCGGAGATATCATGATGGTAAGGTCTCTGGCAGCGGAATGGGCGCA
GTACGGGATTACGGTAAATGGAATCGGACCAGGATATTTTCCAACGGAT
ATCGACAAAGAATATCTGGCGACGGATTATTTTAAAGGACAGTTGGCCA
TGCATTGCCCGATGGGCAGGATTGGACGGGACGGCGAATTGAATGGAGT
CTTGATCTATTTTGCATCGGATGCATCCAGTTATACGACCGGACAGATC
ATGTATGTAGATGGCGGCTGGACGCTTGTTTAG
(SEQ ID NO: 2)
MKNLFDLTGKVALITGASSGLGVQMAQGLAGQGAKLAIVARRMDRLEKL
AKEFEDNGTECLPVKCDITKEEEIIEMVDKVISHYGQIDILVNNAGMAS
GTASEDMTLEEWDKIIRLNLTGSFLVSREVGKHMIASRYGKIINTCSIQ
GIRCTMGMPGTPYNSSKGGDIMMVRSLAAEWAQYGITVNGIGPGYFPTD
IDKEYLATDYFKGQLAMHCPMGRIGRDGELNGVLIYFASDASSYTTGQI
MYVDGGWTLV
DesG from Propionimicrobium lymphophilum
(SEQ ID NO: 3)
ATGAGTAGATTTTCAGGCAAAATCGCAGTAGTCACCGGTGCTAGTTCCG
GCATGGGAAAAGAGATCGCCCGGTCTTTGTGCGAGGAAGGGGCTACGGT
AGTAGCGGTGGCGCGTCGTCTAAACAGACTCGAAGAGCTGGCTGAAAAG
TGTAAGAACGCAGAGGGGCAGATTATCCCTTTCAGGGCTGATCTCATGA
ATGACGATGAGAACCGAAAAATGATTGAGTTCGCCGTCGAGACAGGTGG
AAAACTCGATATCCTCGTCAACAATGCGGGCATGATGGACGAGATGAAG
CCTGTTAGCGAAATCGACGAAGAGCTCTACGACAAAGTCATGACTCTTA
ACGCAAAGAGCCCGATGCTCGCCACTCAGAGCGCTGTCTTGCAGATGGA
AAAACAGGAAACTGGCGGCAATATCGTCAACGTAGCTTCTATTGGCGGC
ACTAACGGGTGCAAAGCTGGAGTGGTTTACACAATGTCGAAGCACGCAT
TGGTTGGGCTCACCAAAAATACGGCCTTTATGTATGTTGGAAAGAACAT
TCGTTGCAATGCTGTGTGCCCGGGTGGCGTGAAGACTGAGGTAGACATC
AACATGACGGCACCAAGCCAGCTTGGCCTGGAGAGGGTCATGACTGGAG
TCGATACCGGTATCAGGCAAGCTGAGGTTGAGGAAATTTCACCGTTGGT
GTTGTTCCTGGCAAGTGATGATGCGTCTTTCATTACGGGTGCTGTGGTA
GCAGCAGACGGTGGCATAACCAGCGCGTAA
(SEQ ID NO: 4)
MSRFSGKIAVVTGASSGMGKEIARSLCEEGATVVAVARRLNRLEELAEK
CKNAEGQIIPFRADLMNDDENRKMIEFAVETGGKLDILVNNAGMMDEMK
PVSEIDEELYDKVMTLNAKSPMLATQSAVLQMEKQETGGNIVNVASIGG
TNGCKAGVVYTMSKHALVGLTKNTAFMYVGKNIRCNAVCPGGVKTEVDI
NMTAPSQLGLERVMTGVDTGIRQAEVEEISPLVLFLASDDASFITGAVV
AADGGITSA

### REFERENCES

1. Sender, R., Fuchs, S. & Milo, R. Revised estimates for the number of human and bacteria cells in the body. PLoS Biol 14, e1002533 (2016).
2. Qin, J. et al. A human gut microbial gene catalogue established by metagenomic sequencing. Nature 464, 59-65 (2010).
3. Markle, J. G. et al. Sex differences in the gut microbiome drive hormone-dependent regulation of autoimmunity. Science 339, 1084-1088 (2013).
4. Collden, H. et al. The gut microbiota is a major regulator of androgen metabolism in intestinal contents. American Journal of Physiology-Endocrinology and Metabolism 317, E1182-E1192 (2019).
5. Pernigoni, N. et al. Commensal bacteria promote endocrine resistance in prostate cancer through androgen biosynthesis. Science 374, 216-224 (2021).
6. Ridlon, J. M. et al. Clostridium scindens: A human gut microbe with a high potential to convert glucocorticoids into androgens. J Lipid Res 54, 2437-2449 (2013).
7. Devendran, S., Mythen, S. M. & Ridlon, J. M. The desA and desB genes from Clostridium scindens ATCC 35704 encode steroid-17,20-desmolase. J Lipid Res 59, 1005-1014 (2018).
8. Bernardi, R. C. et al. Bacteria on steroids: the enzymatic mechanism of an NADH-dependent dehydrogenase that regulates the conversion of cortisol to androgen in the gut microbiome. BioRxiV, 2020.2006. 2012.149468 (2020).
9. Pretorius, E. et al. 11-Ketotestosterone and 11-Ketodihydrotestosterone in Castration Resistant Prostate Cancer: Potent Androgens Which Can No Longer Be Ignored. PLoS One 11, e0159867 (2016).
10. Buttigliero, C. et al. Understanding and overcoming the mechanisms of primary and acquired resistance to abiraterone and enzalutamide in castration resistant prostate cancer. Cancer Treat Rev 41, 884-892 (2015).
11. Montgomery, R. B. et al. Maintenance of intratumoral androgens in metastatic prostate cancer: a mechanism for castration-resistant tumor growth. Cancer Res 68, 4447-4454 (2008).
12. Titus, M. A., Schell, M. J., Lih, F. B., Tomer, K. B. & Mohler, J. L. Testosterone and dihydrotestosterone tissue levels in recurrent prostate cancer. Clin Cancer Res 11, 4653-4657 (2005).
13. Pretorius, E., Arlt, W. & Storbeck, K. H. A new dawn for androgens: Novel lessons from 11-oxygenated C19 steroids. Mol Cell Endocrinol 441, 76-85 (2017).
14. Swart, A. C. & Storbeck, K. H. 11β-Hydroxyandrostenedione: Downstream metabolism by 11βHSD, 17PHSD and SRD5A produces novel substrates in familiar pathways. Mol Cell Endocrinol 408, 114-123 (2015).
15. Barnard, M., Mostaghel, E. A., Auchus, R. J. & Storbeck, K. H. The role of adrenal derived androgens in castration resistant prostate cancer. J Steroid Biochem Mol Biol 197, 105506 (2020).
16. Turcu, A. F., Rege, J., Auchus, R. J. & Rainey, W. E. 11-Oxygenated androgens in health and disease. Nature Reviews Endocrinology 16, 284-296 (2020).
17. Ly, L. K. et al. Bacterial steroid-17,20-desmolase is a taxonomically rare enzymatic pathway that converts prednisone to 1,4-androstanediene-3,11,17-trione, a metabolite that causes proliferation of prostate cancer cells. J Steroid Biochem Mol Biol 199, 105567 (2020).
18. de Prada, P., Setchell, K. D. & Hylemon, P. B. Purification and characterization of a novel 17α-hydroxysteroid dehydrogenase from an intestinal Eubacterium sp. VPI 12708. J Lipid Res 35, 922-929 (1994).
19. Schiffer, L. et al. Human steroid biosynthesis, metabolism and excretion are differentially reflected by serum and urine steroid metabolomes: A comprehensive review. J Steroid Biochem Mol Biol 194, 105439 (2019).
20. Dehennin, L. Secretion by the human testis of epitestosterone, with its sulfoconjugate and precursor androgen 5-androstene-3 beta,17 alpha-diol. J Steroid Biochem Mol Biol 44, 171-177 (1993).
21. Doden, H. L. & Ridlon, J. M. Microbial hydroxysteroid dehydrogenases: From alpha to omega. Microorganisms 9 (2021).
22. Jumper, J. et al. Highly accurate protein structure prediction with AlphaFold. Nature 596, 583-589 (2021).
23. Humphrey, W. VMD-visual molecular dynamics. Journal of molecular graphics 14, 33-38 (1996).
24. Ribeiro, J. V. et al. QwikMD-integrative molecular dynamics toolkit for novices and experts. Scientific reports 6, 26536 (2016).
25. Phillips, J. C. et al. Scalable molecular dynamics on CPU and GPU architectures with NAM D. The Journal of chemical physics 153 (2020).
26. Bellemare, V., Faucher, F., Breton, R. & Luu-The, V. Characterization of 17α-hydroxysteroid dehydrogenase activity (17α-HSD) and its involvement in the biosynthesis of epitestosterone. BMC Biochem 6, 12 (2005).
27. Fernandez-Materan, F. V. et al. Genome sequences of nine Clostridium scindens strains isolated from human feces. Microbiology Resource Announcements (submitted)
28. Olivos Caicedo, K. Y. et al. Complete genome sequence of the archetype bile acid 7α-dehydroxylating bacterium, Clostridium scindens VP112708, isolated from human feces, circa 1980. Microbiology Resource Announcements 12, e00029-00023 (2023).
29. Olivos Caicedo, K. Y. et al. Pangenome analysis of Clostridium scindens: a diverse bile acid-metabolizing commensal gut bacterium Gut Microbes (submitted)
30. Maucher, A., von Angerer, E., Hampl, R. & Stárka, L. The activity of epitestosterone in hormone dependent prostate tumour models. Endocr Regul 28, 23-29 (1994).
31. Schiffer, L., Arlt, W. & Storbeck, K. H. 5α-reduction of epitestosterone is catalysed by human SRD5A1 and SRD5A2 and increases androgen receptor transactivation. J Steroid Biochem Mol Biol 241, 106516 (2024).
32. Lapcik, O., Hampl, R., Hill, M. & Sterka, L. Plasma levels of epitestosterone from prepuberty to adult life. J Steroid Biochem Mol Biol 55, 405-408 (1995).
33. Veldscholte, J. et al. The androgen receptor in LNCaP cells contains a mutation in the ligand binding domain which affects steroid binding characteristics and response to antiandrogens. J Steroid Biochem Mol Biol 41, 665-669 (1992).
34. Tran, C. et al. Development of a second-generation antiandrogen for treatment of advanced prostate cancer. Science 324, 787-790 (2009).
35. Lawrence, M. G., Lai, J. & Clements, J. A. Kallikreins on steroids: structure, function, and hormonal regulation of prostate-specific antigen and the extended kallikrein locus. Endocr Rev 31, 407-446 (2010).
36. Sfanos, K. S. et al. A molecular analysis of prokaryotic and viral DNA sequences in prostate tissue from patients with prostate cancer indicates the presence of multiple and diverse microorganisms. Prostate 68, 306-320 (2008).
37. Doden, H. L. et al. Structural and biochemical characterization of 20β-hydroxysteroid dehydrogenase from Bifidobacterium adolescentis strain L2-32. J Biol Chem 294, 12040-12053 (2019).
38. Jeong, Y. & Irudayaraj, J. Hierarchical encapsulation of bacteria in functional hydrogel beads for inter- and intra-species communication. Acta Biomater 158, 203-215 (2023).
39. Jeong, Y., Kong, W., Lu, T. & Irudayaraj, J. Soft hydrogel-shell confinement systems as bacteria-based bioactuators and biosensors. Biosens Bioelectron 219, 114809 (2023).
40. Devendran, S., Mendez-Garcia, C. & Ridlon, J. M. Identification and characterization of a 20β-HSDH from the anaerobic gut bacterium Butyricicoccus desmolans ATCC 43058. J Lipid Res 58, 916-925 (2017).
41. Shrestha, E. et al. Profiling the Urinary Microbiome in Men with Positive versus Negative Biopsies for Prostate Cancer. J Urol 199, 161-171 (2018).
42. Goncalves, M. F. M. et al. Microbiota of Urine, Glans and Prostate Biopsies in Patients with Prostate Cancer Reveals a Dysbiosis in the Genitourinary System. Cancers (Basel) 15, 1423 (2023).
43. Valentin López, J. C., Lange, C. A. & Dehm, S. M. Androgen receptor and estrogen receptor variants in prostate and breast cancers. J Steroid Biochem Mol Biol 241, 106522 (2024).
44. O'Reilly, M. W. et al. 11-Oxygenated C19 steroids are the oredominant androgens in polycystic ovary syndrome. J Clin Endocrinol Metab 102, 840-848 (2017).
45. Andrews, S. FastQC: a quality control tool for high throughput sequence data. bioinformatics.babraham.ac.uk/projects/fastqc. (2010).
46. Shen, W., Le, S., Li, Y. & Hu, F. SeqKit: a cross-platform and ultrafast toolkit for FASTA/Q file manipulation. PloS One 11, e0163962 (2016).
47. Bolger, A. M., Lohse, M. & Usadel, B. Trimmomatic: a flexible trimmer for Illumina sequence data. Bioinformatics 30, 2114-2120 (2014).
48. Kopylova, E., Noé, L. & Touzet, H. SortMeRNA: fast and accurate filtering of ribosomal RNAs in metatranscriptomic data. Bioinformatics 28, 3211-3217 (2012).
49. Patro, R., Duggal, G., Love, M. I., Irizarry, R. A. & Kingsford, C. Salmon provides fast and bias-aware quantification of transcript expression. Nature Methods 14, 417-419 (2017).
50. Robinson, M. D., McCarthy, D. J. & Smyth, G. K. edgeR: a Bioconductor package for differential expression analysis of digital gene expression data. Bioinformatics 26, 139-140 (2010).
51. Ritchie, M. E. et al. limma powers differential expression analyses for RNA-sequencing and microarray studies. Nucleic Acids Research 43, e47-e47 (2015).
52. Rappsilber, J., Mann, M. & Ishihama, Y. Protocol for micro-purification, enrichment, pre-fractionation and storage of peptides for proteomics using StageTips. Nature Protocols 2, 1896-1906 (2007).
53. Peiffer, L. B. et al. Composition of gastrointestinal microbiota in association with treatment response in individuals with metastatic castrate resistant prostate cancer progressing on enzalutamide and initiating treatment with anti-PD-1 (pembrolizumab). Neoplasia 32, 100822 (2022).
54. Jeong, Y., Ahmad, S. & Irudayaraj, J. Dynamic Effect of β-Lactam Antibiotic Inactivation Due to the Inter- and Intraspecies Interaction of Drug-Resistant Microbes. ACS Biomater Sci Eng 10, 1461-1472 (2024).
55. Kolmogorov, M., Yuan, J., Lin, Y. & Pevzner, P. A. Assembly of long, error-prone reads using repeat graphs. Nature Biotechnology 37, 540-546 (2019).
56. Gurevich, A., Saveliev, V., Vyahhi, N. & Tesler, G. QUAST: quality assessment tool for genome assemblies. Bioinformatics 29, 1072-1075 (2013).
57. Seppey, M., Manni, M. & Zdobnov, E. M. BUSCO: assessing genome assembly and annotation completeness. Gene Prediction: methods and protocols, 227-245 (2019).
58. Seemann, T. Prokka: rapid prokaryotic genome annotation. Bioinformatics 30, 2068-2069 (2014).
59. Grant, J. R. & Stothard, P. The CGView Server: a comparative genomics tool for circular genomes. Nucleic Acids Research 36, W181-W184 (2008).
60. Pritchard, L., Glover, R. H., Humphris, S., Elphinstone, J. G. & Toth, I. K. Genomics and taxonomy in diagnostics for food security: soft-rotting enterobacterial plant pathogens. Analytical Methods 8, 12-24 (2016).
61. Marçais, G. et al. MUMmer4: A fast and versatile genome alignment system. PLoS Computational Biology 14, e1005944 (2018).
62. Page, A. J. et al. Roary: rapid large-scale prokaryote pan genome analysis. Bioinformatics 31, 3691-3693 (2015).
63. Darriba, D. et al. ModelTest-NG: a new and scalable tool for the selection of DNA and protein evolutionary models. Molecular Biology and Evolution 37, 291-294 (2020).
64. Kozlov, A. M., Darriba, D., Flouri, T., Morel, B. & Stamatakis, A. RAxML-NG: a fast, scalable and user-friendly tool for maximum likelihood phylogenetic inference. Bioinformatics 35, 4453-4455 (2019).
65. Letunic, I. & Bork, P. Interactive Tree Of Life (iTOL) v5: an online tool for phylogenetic tree display and annotation. Nucleic Acids Research 49, W293-W296 (2021).
66. Shimoyama, Y. pyGenomeViz: A genome visualization python package for comparative genomics. pypi.org/(2022).
67. Yan, L. Package “ggvenn.” rbasics.org/packages/ggvenn-package-in-r/(2021).
68. Zeng, S. et al. A compendium of 32,277 metagenome-assembled genomes and over 80 million genes from the early-life human gut microbiome. Nature Communications 13, 5139 (2022).
69. Wilkinson, T. et al. 1200 high-quality metagenome-assembled genomes from the rumen of African cattle and their relevance in the context of sub-optimal feeding. Genome Biology 21, 1-25 (2020).
70. Almeida, A. et al. A unified catalog of 204,938 reference genomes from the human gut microbiome. Nature Biotechnology 39, 105-114 (2021).
71. Tamburini, F. B. et al. Short- and long-read metagenomics of urban and rural South African gut microbiomes reveal a transitional composition and undescribed taxa. Nature Communications 13, 926 (2022).
72. Pasolli, E. et al. Extensive unexplored human microbiome diversity revealed by over 150,000 genomes from metagenomes spanning age, geography, and lifestyle. Cell 176, 649-662. e620 (2019).
73. Bryan, D. M. et al. Ultra-deep sequencing of Hadza Hunter-Gatherers recovers vanishing gut microbes. bioRxiv, 2022.2003.2030.486478 (2022).
74. Lemos, L. N. et al. Large scale genome-centric metagenomic data from the gut microbiome of food-producing animals and humans. Sci Data 9, 366 (2022).
75. Gounot, J.-S. et al. Genome-centric analysis of short and long read metagenomes reveals uncharacterized microbiome diversity in Southeast Asians. Nature Communications 13, 6044 (2022).
76. Robert, C. E. MUSCLE v5 enables improved estimates of phylogenetic tree confidence by ensemble bootstrapping. bioRxiv, 2021.2006.2020.449169 (2021).
77. Castresana, J. Selection of conserved blocks from multiple alignments for their use in phylogenetic analysis. Mol Biol Evol 17, 540-552 (2000).
78. Minh, B. Q. et al. Corrigendum to: IQ-TREE 2: New Models and Efficient Methods for Phylogenetic Inference in the Genomic Era. Mol Biol Evol 37, 2461 (2020).
79. Stöver, B. C. & Muller, K. F. TreeGraph 2: combining and visualizing evidence from different phylogenetic analyses. BMC Bioinformatics 11, 7 (2010).
80. Huson, D. H. & Scornavacca, C. Dendroscope 3: an interactive tool for rooted phylogenetic trees and networks. Syst Biol 61, 1061-1067 (2012).
81. Marru, S. et al. in Practice and Experience in Advanced Research Computing 26-34 (2023).
82. Ŝali, A. & Blundell, T. L. Comparative protein modelling by satisfaction of spatial restraints. Journal of molecular biology 234, 779-815 (1993).
83. Laskowski, R. A., MacArthur, M. W., Moss, D. S. & Thornton, J. M. PROCHECK: a program to check the stereochemical quality of protein structures. Journal of applied crystallography 26, 283-291 (1993).
84. Colovos, C. & Yeates, T. O. Verification of protein structures: patterns of nonbonded atomic interactions. Protein science 2, 1511-1519 (1993).
85. Altschul, S. F. et al. Gapped BLAST and PSI-BLAST: a new generation of protein database search programs. Nucleic Acids Research 25, 3389-3402 (1997).
86. Spivak, M. et al. VMD as a platform for interactive small molecule preparation and visualization in quantum and classical simulations. Journal of Chemical Information and Modeling 63, 4664-4678 (2023).
87. Scheurer, M. et al. PyContact: rapid, customizable, and visual analysis of noncovalent interactions in MD simulations. Biophysical journal 114, 577-583 (2018).
88. Darden, T., York, D. & Pedersen, L. Particle mesh Ewald: An N-log (N) method for Ewald sums in large systems. The Journal of chemical physics 98, 10089-10092 (1993).
89. Melo, M. C., Bernardi, R. C., De La Fuente-Nunez, C. & Luthey-Schulten, Z. Generalized correlation-based dynamical network analysis: a new high-performance approach for identifying allosteric communications in molecular dynamics trajectories. The Journal of Chemical Physics 153 (2020).
90. Team, R. C. R: A language and environment for statistical computing. R Foundation for Statistical Computing. r-project.org/(2013). fied and named desG.

1. An isolated nucleic acid molecule comprising:
(a) a first polynucleotide as set forth in SEQ ID NO:1 and/or SEQ ID NO:3; and
(i) a second heterologous polynucleotide; or
(ii) a detectable label;
(b) a sequence set forth in SEQ ID NO:1 and/or SEQ ID NO:3 and containing 1 to 40 nucleic acid substitution modifications relative to SEQ ID NO:1 and/or SEQ ID NO:3; or
(c) a sequence set forth in SEQ ID NO:1 and/or SEQ ID NO:3.

2. The isolated nucleic acid molecule of claim 1, wherein the second heterologous polynucleotide encodes a marker, a label, or purification tag or comprises a heterologous expression control sequence.

3. (canceled)

4. (canceled)

5. The isolated nucleic acid molecule of claim 1, wherein the nucleic acid substitution modifications result in conservative amino acid substitution modifications, or semi-conservative substitution modification, or combinations thereof in a polypeptide expressed from the isolated nucleic acid molecule.

6. An expression cassette comprising the isolated nucleic acid molecule of claim 1 and a second heterologous polynucleotide comprising at least one expression control sequence.

7. (canceled)

8. (canceled)

9. A vector comprising the expression cassette of claim 6.

10. An isolated polypeptide comprising:
(i) a sequence encoded by the isolated nucleic acid molecule of claim 1; or
(ii) a sequence set forth in SEQ ID NO:2 or SEQ ID NO:4 and containing 1 to 20 amino acid substitution modifications relative to SEQ ID NO:2 or SEQ ID NO:4.

11. The isolated polypeptide of claim 10, wherein the polypeptide has dehydrogenase 17α-HSDH activity (SEQ ID NO:2) or 17β3-HSDH activity (SEQ ID NO:4).

12. (canceled)

13. An isolated polypeptide comprising a sequence set forth in SEQ ID NO:2 and/or SEQ ID NO:4 and an indicator reagent, an amino acid spacer, an amino acid linker, a signal sequence, a stop transfer sequence, a transmembrane domain, a protein purification ligand, an affinity purification tag, a heterologous polypeptide, or a combination thereof.

14. (canceled)

15. A recombinant cell comprising the isolated nucleic acid of claim 1.

16. (canceled)

17. The recombinant cell of claim 15, wherein the recombinant cell is a bacterial cell, a fungal cell, or a eukaryotic cell.

18. (canceled)

19. (canceled)

20. A method of producing 17α-hydroxysteroid dehydrogenase (17α-HSDH) comprising culturing the recombinant cell of claim 15, wherein the isolated nucleic acid molecule is as set forth in SEQ ID NO:1, and recovering 17α-hydroxysteroid dehydrogenase.

21. A method of producing 17β3-hydroxysteroid dehydrogenase (17β3-HSDH) comprising culturing the recombinant cell of claim 15, wherein the isolated nucleic acid molecule is as set forth in SEQ ID NO:3, and recovering 17β-hydroxysteroid dehydrogenase.

22. A method of producing epitestosterone comprising contacting the recombinant cell of claim 15, wherein the isolated nucleic acid molecule is as set forth in SEQ ID NO:1, with androstenedione and recovering epitestosterone.

23. The method of claim 22, wherein the recombinant cell expresses androstenedione naturally or recombinantly.

24. A method of producing testosterone comprising contacting the recombinant cell of claim 15, wherein the isolated nucleic acid molecule is as set forth in SEQ ID NO:3, with androstenedione and recovering testosterone.

25. The method of claim 24, wherein the recombinant cell expresses androstenedione naturally or recombinantly.

26. A method of identifying prostate cancer, resistant prostate cancer, or advancing prostate cancer in a patient comprising detecting a level of 17α-hydroxysteroid dehydrogenase (17α-HSDH), detecting a level of 17β-hydroxysteroid dehydrogenase (17β-HSDH), or detecting both a level of 17α-HSDH and 17β-HSDH present in a prostatectomy sample, a urine sample, or a fecal sample of the patient, wherein an elevated level of 17α-HSDH as compared to a control sample or standard indicates prostate cancer, resistant prostate cancer, or advancing prostate cancer.

27. The method of claim 26, wherein the 17α-hydroxysteroid dehydrogenase is as set forth in SEQ ID NO:2.

28. The method of claim 26, wherein the 17 β-hydroxysteroid dehydrogenase is as set forth in SEQ ID NO:4.

29. The method of claim 26, wherein the patient is treated for prostate cancer by prostatectomy, hormone therapy, active surveillance, radiation therapy, high-intensity focused ultrasound, cryotherapy, chemotherapy, immunotherapy, and/or bisphosphonate therapy.

30. (canceled)

31. (canceled)

32. (canceled)


SEQUENCE LISTING (14 sequences)

SEQ ID NO: 1 - DNA, 768 bp, Clostridium scindens
  source 1..768: mol_type = genomic DNA
atgaagaatttatttgatctgacagggaaggtggcgctgatcaccggcgcttcttctggacttggagtacaaatggcccaaggactcgcggggcagggcgccaaactggcgattgtggccaggaggatggatcgcctggaaaagctggcgaaagaattcgaggataacggtactgagtgtctgcctgtgaagtgtgatattacgaaggaagaagagatcattgaaatggtagataaggtgatatcccactatgggcagatagacattctggtgaataatgcaggaatggcatctggaactgcatcggaggatatgacgctggaggaatgggataagattatacggctgaatctgaccggatcgtttcttgtatcccgggaagtagggaagcatatgattgccagccgatatggaaagattattaatacctgctccatccagggaatccgatgtaccatggggatgccgggaactccttacaattcgtcaaaaggcggagatatcatgatggtaaggtctctggcagcggaatgggcgcagtacgggattacggtaaatggaatcggaccaggatattttccaacggatatcgacaaagaatatctggcgacggattattttaaaggacagttggccatgcattgcccgatgggcaggattggacgggacggcgaattgaatggagtcttgatctattttgcatcggatgcatccagttatacgaccggacagatcatgtatgtagatggcggctggacgcttgtttag

SEQ ID NO: 2 - protein, 255 aa, Clostridium scindens
MKNLFDLTGKVALITGASSGLGVQMAQGLAGQGAKLAIVARRMDRLEKLAKEFEDNGTECLPVKCDITKEEEIIEMVDKVISHYGQIDILVNNAGMASGTASEDMTLEEWDKIIRLNLTGSFLVSREVGKHMIASRYGKIINTCSIQGIRCTMGMPGTPYNSSKGGDIMMVRSLAAEWAQYGITVNGIGPGYFPTDIDKEYLATDYFKGQLAMHCPMGRIGRDGELNGVLIYFASDASSYTTGQIMYVDGGWTLV

SEQ ID NO: 3 - DNA, 765 bp, Propionimicrobium lymphophilum
  source 1..765: mol_type = genomic DNA
atgagtagattttcaggcaaaatcgcagtagtcaccggtgctagttccggcatgggaaaagagatcgcccggtctttgtgcgaggaaggggctacggtagtagcggtggcgcgtcgtctaaacagactcgaagagctggctgaaaagtgtaagaacgcagaggggcagattatccctttcagggctgatctcatgaatgacgatgagaaccgaaaaatgattgagttcgccgtcgagacaggtggaaaactcgatatcctcgtcaacaatgcgggcatgatggacgagatgaagcctgttagcgaaatcgacgaagagctctacgacaaagtcatgactcttaacgcaaagagcccgatgctcgccactcagagcgctgtcttgcagatggaaaaacaggaaactggcggcaatatcgtcaacgtagcttctattggcggcactaacgggtgcaaagctggagtggtttacacaatgtcgaagcacgcattggttgggctcaccaaaaatacggcctttatgtatgttggaaagaacattcgttgcaatgctgtgtgcccgggtggcgtgaagactgaggtagacatcaacatgacggcaccaagccagcttggcctggagagggtcatgactggagtcgataccggtatcaggcaagctgaggttgaggaaatttcaccgttggtgttgttcctggcaagtgatgatgcgtctttcattacgggtgctgtggtagcagcagacggtggcataaccagcgcgtaa

SEQ ID NO: 4 - protein, 254 aa, Propionimicrobium lymphophilum
MSRFSGKIAVVTGASSGMGKEIARSLCEEGATVVAVARRLNRLEELAEKCKNAEGQIIPFRADLMNDDENRKMIEFAVETGGKLDILVNNAGMMDEMKPVSEIDEELYDKVMTLNAKSPMLATQSAVLQMEKQETGGNIVNVASIGGTNGCKAGVVYTMSKHALVGLTKNTAFMYVGKNIRCNAVCPGGVKTEVDINMTAPSQLGLERVMTGVDTGIRQAEVEEISPLVLFLASDDASFITGAVVAADGGITSA

SEQ ID NO: 5 - DNA, 32 bp, synthetic construct
  source 1..32: mol_type = other DNA
atatatggatccgatgaagaatttatttgatc

SEQ ID NO: 6 - DNA, 30 bp, synthetic construct
  source 1..30: mol_type = other DNA
atatataagcttctaaacaagcgtccagcc

SEQ ID NO: 7 - DNA, 20 bp, synthetic construct
  source 1..20: mol_type = other DNA
gagtacaaatggcccaagga

SEQ ID NO: 8 - DNA, 22 bp, synthetic construct
  source 1..22: mol_type = other DNA
gcagacactcagtaccgttatc

SEQ ID NO: 9 - DNA, 19 bp, synthetic construct
  source 1..19: mol_type = other DNA
caacgcgwrgaaccttacc

SEQ ID NO: 10 - DNA, 17 bp, synthetic construct
  source 1..17: mol_type = other DNA
cracacgagctgacgac

SEQ ID NO: 11 - DNA, 18 bp, synthetic construct
  source 1..18: mol_type = other DNA
cgtgacgtggattggtgc

SEQ ID NO: 12 - DNA, 18 bp, synthetic construct
  source 1..18: mol_type = other DNA
actgccctgccacgagag

SEQ ID NO: 13 - DNA, 20 bp, synthetic construct
  source 1..20: mol_type = other DNA
tcaaggctgagaacgggaag

SEQ ID NO: 14 - DNA, 20 bp, synthetic construct
  source 1..20: mol_type = other DNA
tggactccacgacgtactca